(19) United States
(12) Patent Application Publication
(10) Pub. No.: US 2010/0216187 A1
(43) Pub. Date: Aug. 26, 2010

(54) AMINO ACID SEQUENCES THAT BIND TO A DESIRED MOLECULE IN A CONDITIONAL MANNER

(75) Inventors: Ignace Joseph Isabella Lasters, Antwerpen (BE); Hendricus Renerus Jacobus Mattheus Hoogenboom, Maastricht (NL)

Correspondence Address: WOLF GREENFIELD & SACKS, P.C., 600 ATLANTIC AVENUE, BOSTON, MA, 02210-2206, US

(73) Assignee: ABLYNX N.V., GHENT-ZWIJNAARDE (BE)

(21) Appl. No.: 12/311,768

(22) PCT Filed: Oct. 11, 2007
(86) PCT No.: PCT/EP2007/060850

§ 371 Date: Feb. 22, 2010

(30) Foreign Application Priority Data
Oct. 11, 2006 (US) 60/850775

(51) Int. Cl. C12P 21/00; C07K 14/00; C07K 16/00; C07H 21/00; C12N 5/10; C07K 2/00

(52) U.S. Cl. 435/69.1; 530/350; 530/387.3; 536/23.1; 435/325; 530/300

(57) ABSTRACT

The present invention relates to amino acid sequences that bind to serum proteins such as serum albumin; to compounds, proteins and polypeptides comprising or essentially consisting of such amino acid sequences; to nucleic acids that encode such amino acid sequences, proteins or polypeptides; to compositions, and in particular pharmaceutical compositions, that comprise such amino acid sequences, proteins and polypeptides; and to uses of such amino acid sequences, proteins and polypeptides, is essentially conditional on different physiological situations, e.g. is different under acidic condition than under pH-neutral condition.

The present invention relates to amino acid sequences that bind to a desired molecule in a conditional manner (as defined herein), to proteins and polypeptides comprising or essentially consisting of such amino acid sequences; to nucleic acids that encode such amino acid sequences, proteins or polypeptides; to compositions, and in particular pharmaceutical compositions, that comprise such amino acid sequences, proteins and polypeptides; and to uses of such amino acid sequences, proteins and polypeptides.

Other aspects, embodiments, advantages and applications of the invention will become clear from the further description herein.

Proteins and peptides that bind to desired molecules are well known in the art. Some non-limiting examples include peptides and proteins with an immunoglobulin fold (i.e. immunoglobulins), such as antibodies and antibody fragments, binding units and binding molecules derived from antibodies and antibody fragments (such as heavy chain variables domains, light chain variable domains, domain antibodies and proteins and peptides suitable for use as domain antibodies, single domain antibodies and proteins and peptides suitable for use as single domain antibodies, Nanobodies® and dAbs™; as well as suitable fragments of any of the foregoing), as well as constructs comprising such antibody fragments, binding units or binding molecules (such as scFvs and diabodies). Reference is made to the prior art cited herein.

Other binding units or binding molecules for example include, without limitation, molecules based on other protein scaffolds than immunoglobulins including but not limited to protein A domains, tendamistat, fibronectin, lipocalin, CTLA-4, T-cell receptors, designed ankyrin repeats and PDZ domains (Binz et al, Nat. Biotech 2005, Vol 23:1257), and binding moieties based on DNA or RNA including but not limited to DNA or RNA aptamers (Ulrich et al. Comb Chem High Throughput Screen 2006 9(8):619-32).

In a first aspect, the invention relates to an amino acid sequence (also referred to herein as: “an amino acid sequence of the invention”) that is directed against a desired molecule, wherein said amino acid sequence:
a) binds to a desired molecule under a first biological condition with a dissociation constant (K_{D}) of 10⁻⁵ moles/liter or less; and
b) binds to said desired molecule under a second biological condition with a dissociation constant (K_{D}) that is at least 10 fold different from (and in particular more than) the dissociation constant with which said amino acid sequence binds to said desired molecule under said first biological condition.

The invention also relates to compounds (as defined herein) that comprise at least one amino acid sequence of the invention. Such compounds are also referred to herein as “compounds of the invention”)

Other aspects and embodiments of the invention will become clear from the further description herein.

In the present description and claims, the term “biological condition” refers to the condition (or set of conditions) that may occur in the body (e.g. in at least one cell, tissue, organ or biological fluid, such as blood or lymphatic fluid) of an animal (and in particular of a mammal, such as a mouse, rat, rabbit, dog or primate) or human being, which may be a healthy animal or human being or an animal or human being that is suffering from a disease or disorder. The term “biological condition” also encompasses the conditions of in vitro or cellular assays or models that correspond to and/or are representative for conditions that may occur in the body of an animal or human being. Such conditions (whether occurring in vivo in a human or animal body or ex vivo in an in vitro or cellular assay or model) will be clear to the skilled person.

It will also be clear from the disclosure herein that the “first biological condition” will differ in at least one respect from the “second biological condition”. For example, the first biological condition may comprise the physiological conditions that are prevalent in a first physiological compartment or fluid, and the second biological condition comprises the physiological conditions that are prevalent in a second physiological compartment or fluid, wherein the first and second physiological compartments or fluids are, under normal physiological conditions, separated by at least one biological membrane such as a cell membrane, a wall of a cellular vesicle or a subcellular compartment, or a wall of a blood vessel.

According to one specific but non-limiting aspect, the amino acid sequence of the invention (or a compound comprising the same) is also capable, in a human or animal body, of crossing said biological membrane and/or is subjected to a biological action or mechanism (such as an active or passive transport mechanism) that allows it to cross said biological membrane, such that the amino acid sequence or compound of the invention goes from the first physiological compartment (where it is exposed to the first biological condition) into the second physiological compartment (where it exposed to the second biological condition).

Thus, according to one specific, but non-limiting aspect of the invention, the first biological condition may comprise the physiological conditions that are prevalent outside at least one cell of a human or animal body (i.e. extracellular conditions, such as the conditions in the immediate surroundings or near vicinity of said cell, and/or in the circulation of the human or animal body), and the second biological condition may comprise the conditions that are prevalent inside said cell (i.e. intracellular conditions) (or vise versa). For example, according to this specific non-limiting aspect of the invention, the second biological condition may comprise the physiological conditions that are prevalent in at least one intracellular or subcellular compartment of a cell (such as an endosomal compartment) of a human or animal body, and the first biological condition may comprise the conditions that are prevalent outside said cell (or vise versa).

According to another specific, but non-limiting aspect of the invention, the first biological condition may comprise the physiological conditions that are prevalent in the circulation (for example in the bloodstream or lymphatic system) of said human or animal body, and the second biological condition may comprise the conditions that are prevalent in at least one tissue or cell (such as in at least one subcellular compartment of such a cell, such as an endosomal compartment) of a human or animal body (or vise versa).

According to one particular aspect of the present invention, where the amino acid sequence of the invention (as such or bound to the desired molecule) can be taken up (e.g. by internalisation, pinocytosis, transcytosis, endocytosis, phagocytosis or a similar biological mechanism) or has been taken up and is the process of being transferred outside the cell by exocytose or other means by at least one cell of the human or animal body, the first biological condition may comprise the physiological conditions in which the amino acid sequence is present prior to it being taken up by the cell (e.g. outside the cell into which the amino acid sequence of the invention is taken up by internalization or pinocytosis, transcytosis or endocytosis for example in the blood stream or the lymphatic system) and the second biological condition comprises the physiological conditions in which the amino acid sequence is present after the amino acid sequence has been taken up into the cell (for example, in the subcellular compartment in which the amino acid sequence of the invention is present (immediately) upon internalization, pinocytosis, transcytosis or endocytosis (such as an endosome, lysosome, pinosome, or another cellular vesicle); or vise versa.

As will be explained in more detail below, this aspect is of particular importance when the desired molecule is a molecule that is taken up by a cell (i.e. subjected to internalization, pinocytosis, transcytosis or endocytosis or a similar biological mechanism) in the course of recycling thereof, as is for example the case with serum albumin.

Thus, in one specific, but non-limiting aspect, the amino acid sequence is directed against an intended or desired molecule that is subject to recycling and in the course thereof is taken up by at least one cell, and the first biological condition comprises the extracellular conditions with respect to at least one cell of the animal or human body that is involved in recycling of the desired compound (i.e. the conditions that are prevalent outside said cell, such as the conditions at the cell surface or in the immediate surroundings or near vicinity of the cell, and/or the conditions prevalent in the circulation, e.g. in the bloodstream or the lymphatic system), and the second biological condition comprises the conditions that are prevalent inside the cell (i.e. the conditions in the cell or the conditions in one intracellular or subcellular compartment thereof, such as the conditions within an endosome or a vesicle within the cell, and in particular within an intracellular or subcellular compartment that is involved in the recycling of the protein or polypeptide).

As a non-limiting example of this aspect of the invention, the amino acid sequence of the invention may be directed against a serum protein that is subject to recycling by at least one cell of the human or animal body (such as serum albumin), and the first biological condition may comprise the conditions that are prevalent in the circulation of said human or animal body, and the second biological condition may comprise the conditions that are prevalent inside said cell (i.e. the conditions in the cell or the conditions in one intracellular or subcellular compartment thereof, such as the conditions within an endosome or a vesicle within the cell, and in particular within an intracellular or subcellular compartment that is involved in the recycling of the protein or polypeptide).

As another non-limiting example of this aspect of the invention, the amino acid sequence of the invention may be directed against a protein or polypeptide on the surface of a cell that is subject to recycling by said cell (such as a receptor), and the first biological condition may comprise the conditions that are prevalent at the cell surface or in the immediate surroundings of said cell of the animal or human body, and the second biological condition may comprise the conditions that are prevalent inside said cell (i.e. the conditions in the cell or the conditions in one intracellular or subcellular compartment thereof, such as the conditions within an endosome or a vesicle within the cell, and in particular within an intracellular or subcellular compartment that is involved in the recycling of the protein or polypeptide).

This aspect (including the two specific examples thereof) may also allow targeting of the amino acid sequence of the invention (or a compound comprising the same, as further described herein) towards specific cells or tissues into which the desired molecule is taken up by internalization, pinocytosis, transcytosis or endocytosis (whether as part of recycling or otherwise). Outside the cell, the amino acid sequence or compound of the invention will bind to the desired molecule with high affinity or avidity (i.e. with an association constant or dissociation constant as described herein for binding under the first biological condition), and will thus be taken up into the cell while bound to the desired molecule. Upon such internalization, pinocytosis, transcytosis or endocytosis, the affinity or avidity of the amino acid sequence or compound of the invention for the desired compound will be reduced (i.e. to an association constant or dissociation constant as described herein for binding under the second biological condition), so that the amino acid sequence or compound is released from the desired molecule and can perform its intended or desired biological, physiological, pharmaceutical or therapeutic action in the cell. Generally, as will be clear to the skilled person, this mechanism may also be used to allow an amino acid sequence or compound of the invention to cross the cell membrane of a cell and to enter into said cell, and may also be used for intracellular targeting of a compound of the invention.

According to another specific but non-limiting aspect, the first biological condition and the second biological condition may differ in respect of pH, in which said first biological condition may comprise a physiological pH of more than 7.0, for example a pH of more than 7.1 or a pH of more than 7.2, such as a pH in the range of 7.2 to 7.4; and the second biological condition may comprise a physiological pH of less than 7.0, for example a pH of less than 6.7 or a pH of less than 6.5, such as a pH in the range of 6.5 to 6.0 (or vise versa).

According to yet another specific but non-limiting aspect, the first biological condition and the second biological condition may differ in respect of the number and type of proteases. The susceptibility of an amino acid sequence towards protease degradation is highly variable and sequence and protein dependent, the level of protein degradation in vivo will be dependant on the types of proteases actually encountered by the amino acid sequence. For example in endosomes, many cysteine cathepsins are present; in the lysosomes, a large panel of lipases, carbohydrases, proteases and nucleases are present that are optimally active at acidic pH (4.8); in the extracellular space and in the bloodstream, many other proteases (e.g. serine proteases) are active.

According to another specific, but non-limiting aspect, the first and second biological condition differ in respect of any two, any three or essentially all of the following factors: pH, ionic strength, protease contents; in which said factors may be and/or may differ as described herein.

According to another specific, but non-limiting aspect, the first biological condition may comprise the physiological conditions that are prevalent in a first physiological compartment or fluid, and the second biological condition comprises the physiological conditions that are prevalent in a second physiological compartment or fluid, wherein the first and second physiological compartments or fluids are, under normal physiological conditions, separated by at least one biological membrane such as cell membrane, a wall of a cellular vesicle or a subcellular compartment, or a wall of a blood vessel, wherein the conditions prevalent in the first physiological compartment or fluid and the conditions prevalent in the second physiological compartment or fluid differ in respect of any two, any three or essentially all of the following factors: pH, ionic strength, protease contents; in which said factors may be and/or may differ as described herein.

As will be clear from the description herein, the amino acid sequences and compounds of the invention are such that they bind with a different dissociation constant or association constant (which are as defined herein) to their respective desired molecules under the first and second biological conditions, respectively. This is generally referred to herein as “conditional binding”, and amino acid sequences that show such conditional binding are also referred to herein as “conditional” amino acid sequences (such as, for example, “conditional Nanobodies”) or as “conditional binders”.

The conditional amino acid sequences of the invention (as well as compounds comprising the same) are preferably such that they bind to their intended or desired molecule under the second biological condition (or set of biological conditions) with a dissociation constant (K_{D}) that is at least 10 times more, more preferably 100 fold more, more preferably at least 1000 fold more, than the dissociation constant with which the conditional amino acid sequence binds to its intended or desired molecule under the first biological condition (or set of biological conditions); and/or binds to its intended or desired molecule under the second biological condition (or set of biological conditions) with a binding affinity (K_{A}) that is at least 10 times less, more preferably 100 times less, more preferably at least 1000 times less, than the binding affinity with which said amino acid sequence binds to said intended desired molecule under said first biological condition (or set of biological conditions).

Thus, by means of illustration and without limitation, when the amino acid sequences of the invention may bind to said desired molecule under said first second biological condition with a dissociation constant (K_{D}) of about 10⁻⁷ moles/liter and/or with a binding affinity (K_{A}) of about 10⁷ M⁻¹, the amino acid sequences of the invention bind to said desired molecule under said second biological condition with a dissociation constant (K_{D}) of about 10⁻⁶ moles/liter or more and/or with a binding affinity (K_{A}) of about 10⁶ M⁻¹ or less, preferably with a dissociation constant (K_{D}) of about 10⁻⁵ moles/liter or more and/or with a binding affinity (K_{A}) of about 10⁵ M⁻¹ or less, and more preferably with a dissociation constant (K_{D}) of about 10⁻⁴ moles/liter or more and/or with a binding affinity (K_{A}) of about 10⁴ M⁻¹ or less.

In addition, the amino acid sequences and compounds of the invention are preferably such that they bind to said intended or desired molecule under said first biological condition with a dissociation constant (K_{D}) of 10⁻⁶ moles/liter or less, more preferably with a dissociation constant (K_{D}) of 10⁻⁷ moles/liter or less, and even more preferably with a dissociation constant (K_{D}) of 10⁻⁴ moles/liter or less.

Furthermore, the amino acid sequences or compounds of the invention are preferably such that they bind to said intended or desired molecule under said second biological condition with a dissociation constant (K_{D}) of 10⁻⁶ moles/liter or more, more preferably with a dissociation constant (K_{D}) of 10⁻⁵ moles/liter or more, and even more preferably with a dissociation constant (K_{D}) of 10⁻⁴ moles/liter or more.

The dissociation constant may be the actual or apparent dissociation constant, as will be clear to the skilled person. Methods for determining the dissociation constant will be clear to the skilled person, and for example include the techniques mentioned herein. In this respect, it will also be clear that it may not be possible to measure dissociation constants of more then 10⁻⁴ moles/liter or 10⁻³ moles/liter (e.g., of 10⁻² moles/liter). Accordingly, when a dissociation constant cannot be measured, it will be deemed for the purposes of the present invention to be a dissociation constant that is at least 1000 fold more than a dissociation constant of 10⁵ moles/liter.

Optionally, as will also be clear to the skilled person, the (actual or apparent) dissociation constant may be calculated on the basis of the (actual or apparent) association constant (K_{A}), by means of the relationship [K_{D}=1/K_{A}]. For this purpose, methods for determining the association constant at a certain pH value will be clear to the skilled person, and for example include the techniques mentioned herein. Also, from this, it will be clear that the amino acid sequences of the invention may also be such that they bind to the said desired molecule under a second biological condition with a binding affinity (K_{A}) that is at least 10 times less than the binding affinity with which said amino acid sequence binds to said desired molecule under said first biological condition.

The affinity denotes the strength or stability of a molecular interaction. The affinity is commonly given as by the Kd, or dissociation constant, which has units of mol/liter, noted in brief as M. The affinity can also be expressed as an association constant, Ka which equals 1/Kd and has units of (mol/liter)⁻¹, in brief M⁻¹. Throughout this document we will express the stability of molecular interaction by its Kd value. But it should be understood that in view of the relation Ka=1/Kd, specifying the strength of molecular interaction by its Kd value, automatically specifies also the Ka value. The Kd characterizes the strength of a molecular interaction also in a thermodynamic sense as it is related to the free energy (DG) of binding by the well known relation DG=RT.ln(Kd) (equivalently DG=−RT.ln(Ka)), where R equals the gas constant, T equals the absolute temperature and ln denotes the natural logarithm. The Kd of meaningful biological complexes are typically in the range of 10⁻¹⁰M (0.1 nM) to 10⁻⁵M (10000 nM). The stronger an interaction is, the lower is its Kd.

Kd can also be expressed as the ratio of the dissociation rate constant of a complex, denoted as k_{off}, to the rate of its association, denoted kₒₙ. In other words Kd=k_{off}/kₒₙ. Clearly Ka=kₒₙ/k_{off}. The off-rate k_{off} has units s⁻¹ (where s is the SI unit notation of second). The on-rate kₒₙ has units M⁻¹s⁻¹. The on-rate may vary between 10²M⁻¹s⁻¹ to about 10⁷ M⁻¹s⁻¹, approaching the diffusion-limited association rate constant for bimolecular interactions. The off-rate is related to the half-life of a given molecular interaction by the relation t_{1/2}=ln(2)/k_{off}. The off-rate may vary between 10⁻⁶ s⁻¹ (near irreversible complex with a t_{1/2} of multiple days) to 1s⁻¹ (t_{1/2}=0.69 s).

The affinity of a molecular interaction between two molecules can be measured via different techniques such the well the known surface plasmon resonance (SPR) biosensor technique (e.g. Ober et al., Intern. Immunology, 13, 1551-1559, 2001 used a Biacore 3000 SPR biosensor to study the affinity of albumin for FcRn under various pH conditions) where one molecule is immobilized on the biosensor chip and the other molecule is passed over the immobilized molecule under flow conditions yielding kₒₙ, k_{off} measurements and hence Kd (or Ka) values.

It should be noted that the measured Kd corresponds to an apparent Kd if the measuring process somehow influences the intrinsic binding affinity of the implied molecules for example by artifacts related to the coating on the biosensor of one molecule. Also, an apparent Kd may be measured if one molecule contains more than one recognition sites for the other molecule. In such situation the measured affinity may be affected by the avidity of the interaction by the two molecules. For example, SPR experiments with immobilized human FcRn show a significantly higher affinity (avidity) for human IgG as compared to the affinity of the FcRn interaction with immobilized IgG paralleling the 2:1 stoichiometry of the FcRn-IgG interaction (Sanchez et al., Biochemistry, 38, 9471-9476, 1999).

Another approach that may be used to assess affinity is the 2-step ELISA (Enzyme-Linked Immunosorbent Assay) procedure of Friguet et al. (J. Immunol. Methods, 77, 305-19, 1985). This method establishes a solution phase binding equilibrium measurement and avoids possible artifacts relating to adsorption of one of the molecules on a support such as plastic.

For example Nguyen et al. (Protein Eng Des Sel., 19, 291-297, 2006) have recently measured the affinity for albumin of Fab constructs using the Friguet assay. However, the accurate measurement of Kd may be quite labor-intensive and as consequence, often apparent Kd values are determined to assess the binding strength of two molecules. It should be noted that as long all measurements are made in a consistent way (e.g. keeping the assay conditions unchanged) apparent Kd measurements can be used as an approximation of the true Kd and hence in the present document Kd and apparent Kd should be treated with equal importance or relevance.

Finally, it should be noted that in many situations the experienced scientist may judge it to be convenient to determine the binding affinity relative to some reference molecule. For example, to assess the binding strength between molecules A and B, one may e.g. use a reference molecule C that is known to bind to B and that is suitably labeled with a fluorophore or chromophore group or other chemical moiety, such as biotin for easy detection in an ELISA or FACS (Fluorescent activated cell sorting) or other format (the fluorophore for fluorescence detection, the chromophore for light absorption detection, the biotin for streptavidin-mediated ELISA detection). Typically, the reference molecule C is kept at a fixed concentration and the concentration of B is varied for a given concentration or amount of B. As a result an IC50 value is obtained corresponding to the concentration of A at which the signal measured for C in absence of A is halved. Provided Kd_{ref}, the Kd of the reference molecule, is known, as well as the total concentration c_{ref} of the reference molecule, the apparent Kd for the interaction A-B can be obtained from following formula:

Kd=IC50/(1+c_{ref}/Kd_{ref}). Note that if c_{ref}<<Kd_{ref}, Kd≈IC50. Provided one performs the IC50 measurement in a consistent way (e.g. keeping c_{ref} fixed), the strength or stability of a molecular interaction can be assessed by the IC50 and this measurement is judged as equivalent to Kd or to apparent Kd throughout this text.

Preferably, an amino acid sequence of the invention which is in monovalent form (as described herein) will, under the first biological condition, bind to the intended or desired molecule with an affinity (K_{D}) better than 3000 nM, preferably better than 300 nM, more preferably better than 30 nM such as better than 3 nM, and will bind to the intended or desired molecule under the second biological condition with an affinity that is at least 10 times worse, preferably more than 100 times worse, such as at least 1000 times worse or more. For example, and without limitation, a monovalent amino acid sequence may bind to the intended or desired molecule under the second biological condition with an affinity worse than 3 nM, more preferably worse than 30 nM, more preferably worse than 300 nM, such as worse than 3000 nM.

Besides the affinity of the interaction, also the kinetics of the interaction may be a driving factor in the conditional binding behaviour of the molecule. For example differences in on and off-rates may play a role in influencing the outcome of a binding event, e.g. the speed of detachment from a bound antigen upon changing biological conditions, the rate of binding to the antigen upon changing biological conditions. Preferably, an amino acid sequence of the invention which is in monovalent form (as described herein) will, under the first biological condition, bind to the intended or desired molecule with an off-rate better than 10⁻¹ s⁻¹, preferably better than 10⁻² s⁻¹, more preferably better than 10⁻³ s⁻¹ such as better than 10⁻⁴ s⁻¹ and will bind to the intended or desired molecule under the second biological condition with an off-rate that is at least 10 times worse, preferably more than 100 times worse, such as at least 1000 times worse or more. For example, and without limitation, a monovalent amino acid sequence may bind to the intended or desired molecule under the second biological condition with an off-rate worse than 10⁻⁴ s⁻¹, more preferably worse than 10⁻³ s⁻¹, more preferably worse than 10⁻² s⁻¹, such as worse than 10⁻¹ s⁻¹. Preferably, an amino acid sequence of the invention which is in monovalent form (as described herein) will, under the first biological condition, bind to the intended or desired molecule with an on-rate better than 10²M⁻¹ s⁻¹, preferably better than 10³ M⁻¹ s⁻¹, more preferably better than 10⁴ M⁻¹ s⁻¹ such as better than 10⁵ M⁻¹ s⁻¹ and will bind to the intended or desired molecule under the second biological condition with an on-rate that is at least 10 times worse, preferably more than 100 times worse, such as at least 1000 times worse or more. For example, and without limitation, a monovalent amino acid sequence may bind to the intended or desired molecule under the second biological condition with a corresponding on-rate worse than 10⁵ M⁻¹ s⁻¹, more preferably worse than 10⁴ M⁻¹ s⁻¹, more preferably worse than 10³ M⁻¹ s⁻¹, such as worse than 10²M⁻¹ s⁻¹.

In another embodiment of the invention, an amino acid sequence of the invention which may be in monovalent, bivalent or multivalent form (e.g. as described herein) will, under the first biological condition, bind to the intended or desired molecule with k_{off} rate (i.e. an off-rate) between 0.1 s⁻¹ and 10⁻⁶ s⁻¹, preferably between 0.1 s⁻¹ and 10⁻⁵ s⁻¹ and more preferably between 0.01 s⁻¹ and 10⁻⁴ s⁻¹ and said amino acid sequence of the invention will bind to the intended or desired molecule under the second biological condition with an off-rate that is at least 1.5 times higher or more than the off-rate under the first biological condition, preferably more than 1.7 times higher or more than the off-rate under the first biological condition, more preferably 2 times higher or more than the off-rate under the first biological condition, more preferably 3 times higher or more than the off-rate under the first biological condition, more preferably 5 times higher or more than the off-rate under the first biological condition more preferably 10 times higher or more than the off-rate under the first biological condition more preferably 20 times higher or more than the off-rate under the first biological condition.

In another embodiment of the invention, an amino acid sequence of the invention which may be in monovalent, bivalent or multivalent form (e.g. as described herein) will, under the first biological condition, bind to the intended or desired molecule with k_{off} rate (i.e. an off-rate) between 0.1 s⁻¹ and 10⁻⁶ s⁻¹, preferably between 0.1 s⁻¹ and 10⁻⁵ s⁻¹ and more preferably between 0.01 s⁻¹ and 10⁻⁴ s⁻¹ and said amino acid sequence of the invention will bind to the intended or desired molecule under the second biological condition with an off-rate that is at least 1.5 times higher or more than the off-rate under the first biological condition, preferably more than 1.7 times higher or more than the off-rate under the first biological condition, more preferably 2 times higher or more than the off-rate under the first biological condition, more preferably 3 times higher or more than the off-rate under the first biological condition, more preferably 5 times higher or more than the off-rate under the first biological condition more preferably 10 times higher or more than the off-rate under the first biological condition more preferably 20 times higher or more than the off-rate under the first biological condition; and wherein said amino acid sequence of the invention binds monovalently to a serum protein (preferably serum albumin) and to a target protein in monovalent, bivalent or multivalent way.

In another embodiment of the invention, an amino acid sequence of the invention which may be in monovalent, bivalent or multivalent form (e.g. as described herein) will, under the first biological condition, bind to the intended or desired molecule with k_{off} rate (i.e. an off-rate) between 0.1 s⁻¹ and 10⁻⁶ s⁻¹ and said amino acid sequence of the invention will bind to the intended or desired molecule under the second biological condition with an off-rate that is at least 2 times or more than the off-rate under the first biological condition.

In another embodiment of the invention, an amino acid sequence of the invention which may be in monovalent, bivalent or multivalent form (e.g. as described herein) will, under the first biological condition, bind to the intended or desired molecule with k_{off} rate (i.e. an off-rate) between 0.1 s⁻¹ and 10⁻⁶ s⁻¹ and said amino acid sequence of the invention will bind to the intended or desired molecule under the second biological condition with an off-rate that is at least 5 times or more than the off-rate under the first biological condition.

In another embodiment of the invention, an amino acid sequence of the invention which may be in monovalent, bivalent or multivalent form (e.g. as described herein) will, under the first biological condition, bind to the intended or desired molecule with k_{off} rate (i.e. an off-rate) between 0.01 s⁻¹ and 10⁻⁴ s⁻¹ and said amino acid sequence of the invention will bind to the intended or desired molecule under the second biological condition with an off-rate that is at least 2 times higher or more than the off-rate under the first biological condition; and wherein said amino acid sequence of the invention binds monovalently to a serum protein (preferably serum albumin) and to a target protein in a monovalent, bivalent or multivalent way.

In another embodiment of the invention, an amino acid sequence of the invention which may be in monovalent, bivalent or multivalent form (e.g. as described herein) will, under the first biological condition, bind to the intended or desired molecule with k_{off} rate (i.e. an off-rate) between 0.01 s⁻¹ and 10⁻⁴ s⁻¹ and said amino acid sequence of the invention will bind to the intended or desired molecule under the second biological condition with an off-rate that is at least 5 times higher or more than the off-rate under the first biological condition; and wherein said amino acid sequence of the invention binds monovalently to a serum protein (preferably serum albumin) and to a target protein in a monovalent, bivalent or multivalent way.

The binding of an amino acid sequence to an intended or desired molecule (including the association constant, dissociation constant, affinity, kₒₙ rate or k_{off} rate of such binding) under the first and second biological condition, respectively, can be determined in any suitable manner known per se, including, for example, Scatchard analysis and/or competitive binding assays, such as radioimmunoassays (RIA), enzyme immunoassays (ETA) and sandwich competition assays, and the different variants thereof known per se in the art. Again, as mentioned above, the binding can be measured in vivo in the human or animal body, or—where this is not feasible or practicable—ex vivo, for example under the conditions of in vitro or cellular assays or models that correspond to and/or are representative for conditions that may occur in the body of an animal or human body. For example, where the first or second biological condition comprises the physiological conditions prevalent in the circulation of a human or animal, the binding of the amino acid sequence of the invention under said condition can be determined in a blood sample, a plasma sample or another suitable blood- or plasma-derived preparation or solution derived from said human or animal. Where the first or second biological condition comprises the physiological conditions prevalent in a cell, the binding of the amino acid sequence of the invention under said condition can be determined in a suitable cellular extract. Where the first or second biological condition differ in pH and/or in ion strength, the binding of the amino acid sequence of the invention under said first and second biological condition (i.e. at the relevant value(s) of the pH and/or the ionic strength) can for example be determined using one or more suitable physiological buffers or solutions.

The amino acid sequence of the invention may be any protein or polypeptide (or a derivative thereof, such as a pegylated derivative) that can bind to (as described herein) and/or has affinity for an intended or desired molecule.

According to a specific but non-limiting aspect of the invention, the amino acid sequence of the invention may be chosen from the group consisting of proteins and peptides with an immunoglobulin fold, proteins and peptides based on other protein scaffolds then immunoglobulins including but not limited to protein A domains, tendamistat, fibronectin, lipocalin, CTLA-4, T-cell receptors, designed ankyrin repeats and PDZ domains (Binz et al, Nat. Biotech 2005, Vol 23:1257), and binding moieties based on DNA or RNA including but not limited to DNA or RNA aptamers (Ulrich et al. Comb Chem High Throughput Screen 2006 9(8):619-32); and in particular from the group consisting of proteins and peptides with an immunoglobulin fold (or from suitable parts, fragments, analogs, homologs, orthologs, variants, derivatives, etc. of any of the foregoing).

Also, according to one specific, but non-limiting aspect, an amino acid sequence of the invention may comprise or essentially consist of four framework regions separated from each other by three complementarity determining regions (or from suitable parts, fragments, analogs, homologs, orthologs, variants, derivatives, etc. of such proteins or polypeptides. As further described herein, such parts or fragments preferably at least comprise at least one CDR of such a protein or polypeptide). For example, an amino acid sequence of the invention may be chosen from the group consisting of antibodies and antibody fragments, binding units and binding molecules derived from antibodies or antibody fragments, and antibody fragments, binding units or binding molecules; and in particular from the group consisting of heavy chain variable domains, light chain variable domains, domain antibodies and proteins and peptides suitable for use as domain antibodies, single domain antibodies and proteins and peptides suitable for use as single domain antibodies, Nanobodies® and dAbs™ (or from suitable parts, fragments, analogs, homologs, orthologs, variants, derivatives, etc. of such proteins or polypeptides. Again, such parts or fragments preferably at least comprise at least one CDR).

Depending on how the amino acid sequence of the invention is chosen, it preferably comprises between 4 and 500 amino acid residues, more preferably between 5 and 300 amino acid residues, and even more preferably between 10 and 200 amino acid residues, such as between 20 and 150 amino acid residues, for example about 30, 40, 50, 60, 70, 80, 90, 100, 110, 120, 130 or 140 amino acid residues.

Also, the amino acid sequences of the invention preferably comprise a single amino acid chain (with or without disulphide bridges/linkages).

In one specific, non-limiting embodiment, the amino acid sequences of the invention are small linear peptides that essentially do not comprise an immunoglobulin fold. In this embodiment the amino acid sequences of the invention may comprise between 3 and 50, preferably between 5 and 40, such as about 10, 15, 20 or 25 amino acid residues. Such peptides may for example be small synthetic or semi-synthetic peptides and/or may be derived from or comprise at least one CDR from an immunoglobulin of the invention that is directed against the intended or desired molecule (i.e. in which said immunoglobulin may be as further described herein). For example, such a peptide may be derived from or comprise at least one CDR (such as CDR1, CDR2, and in particular CDR3) from a heavy chain variable domain, light chain variable domain, domain antibodies, single domain antibodies, Nanobodies™ or dAbs™ of the invention, and in particular from a Nanobody of the invention. Reference is for example made to WO 03/050531 (Ablynx N.V. and Algonomics N.V.), which describes methods for the identification and selection of peptides, in particular immunoglobulin heavy chain variable domain CDR sequences that bind to a given target or targets of interest.

According to another preferred embodiment, the amino acid sequence of the invention is chosen from the group consisting of domain antibodies, single domain antibodies and proteins and peptides suitable for use as single domain antibodies, Nanobodies® and dAbs™ (or of suitable parts, fragments, analogs, homologs, orthologs, variants, derivatives thereof).

Most preferably, the amino acid sequence of the invention is a V_{HH} domain Nanobody®. For a description of Nanobodies and methods for producing the same, reference is made to the further prior art cited herein.

The intended or desired molecule against which the amino acid sequence of the invention is directed may be any suitable or desired molecule. Generally, it will be a molecule that is present in the body of a human or animal body, for example a molecule that naturally occurs in a human or animal body; a molecule that occurs in a human or animal body when said human or animal suffers from a disease or disorder; or a molecule that does not naturally occur in a human or animal body (but that has been administered or that has otherwise entered into the human or animal body).

When the desired or intended molecule is a molecule that naturally occurs in a human or animal body or on the body of a human or animal that suffers from a disease or disorder, the molecule may for example be any biological molecule, such as a protein, (poly)peptide, receptor, antigen, antigenic determinant, enzyme, factor, etc. Examples of these and other suitable biological molecules will be clear to the skilled person based on the disclosure herein.

When the desired or intended molecule is a molecule that does not occur naturally in a human or animal body, the molecule may for example be a heterologous protein, a (protein present on the coat of) a virus, a (protein present in the cell wall of) a bacterium or fungus, a xenobiotic compound, etc. Examples of these and other suitable biological molecules will be clear to the skilled person based on the disclosure herein.

According to one specific but non-limiting embodiment (described in more detail herein), the intended or desired molecule may be a serum protein such as albumin, and in particular a human serum protein such as human scrum albumin. Examples of other serum proteins against which the present amino acid sequences may be directed are those mentioned in the International application WO 04/003019 (see also EP 1 517 921).

According to one specific but non-limiting embodiment, the intended or desired molecule may be any biological molecule that, within the human or animal body in which it is present, is subjected to recycling, internalization, pinocytosis, transcytosis or endocytosis or otherwise taken up by at least one cell or tissue within the human body. Some non-limiting examples include some serum proteins such as serum albumin and some receptors.

According to one specific but non-limiting embodiment, the intended or desired molecule may be any biological molecule that is present on the surface of at least one cell or tissue of a human or animal body. Again, some non-limiting examples include receptors such as the insulin receptor. According to a specific aspect of this embodiment, this biological molecule can also be taken up by the cell on which it is present, for example as part of recycling (e.g. receptor recycling).

In other aspects, the invention relates to methods for generating the amino acid sequences of the invention. In one aspect, said method at least comprises the steps of:
a) providing a set, collection or library of amino acid sequences; and
b) screening said set, collection or library of amino acid sequences for amino acid sequences that under the first biological condition can bind to said desired molecule with a dissociation constant (K_{D}) of 10⁻⁵ moles/liter or less;
c) screening said set, collection or library of amino acid sequences for amino acid sequences that under said second biological condition bind to said desired molecule with a dissociation constant (K_{D}) that is at least 10 fold more than the dissociation constant with which said amino acid sequence binds to said desired molecule under said first biological condition;
and
d) isolating the amino acid sequence(s) that under the first biological condition can bind to said desired molecule with a dissociation constant (K_{D}) of 10⁻⁵ moles/liter or less and that under said second biological condition bind to said desired molecule with a dissociation constant (K_{D}) that is at least 10 fold more than the dissociation constant with which said amino acid sequence binds to said desired molecule under said first biological condition;

In particular, such a method can comprise the steps of:
a) providing a set, collection or library of amino acid sequences; and
b) screening said set, collection or library of amino acid sequences for amino acid sequences that under the first biological condition can bind to said desired molecule with a dissociation constant (K_{D}) of 10⁻⁵ moles/liter or less, so as to provide a set, collection or library of amino acid sequences that under the first biological condition can bind to said desired molecule with a dissociation constant (K_{D}) of 10⁻⁵ moles/liter or less; and
c) screening the set, collection or library of amino acid sequences obtained in step b) for amino acid sequences that under said second biological condition bind to said desired molecule with a dissociation constant (K_{D}) that is at least 10 fold more than the dissociation constant with which said amino acid sequence binds to said desired molecule under said first biological condition; and

d) isolating the amino acid sequence(s) that under the first biological condition can bind to said desired molecule with a dissociation constant (K_{D}) of 10⁻⁵ moles/liter or less and that under said second biological condition bind to said desired molecule with a dissociation constant (K_{D}) that is at least 10 fold more than the dissociation constant with which said amino acid sequence hinds to said desired molecule under said first biological condition.

Generally, in these methods, the step h) of screening the set, collection or library of amino acid sequences for amino acid sequences that under the first biological condition can bind to said desired molecule with a dissociation constant (K_{D}) of 10⁻⁵ moles/liter or less is performed by screening under the first biological condition.

Similarly, in these methods, the step c) of screening the set, collection or library of amino acid sequences for amino acid sequences that under said second biological condition bind to said desired molecule with a dissociation constant (K_{D}) that is at least 10 fold more than the dissociation constant with which said amino acid sequence binds to said desired molecule under said first biological condition; is performed under the second biological condition.

In other aspects, the invention relates to methods for generating the amino acid sequences of the invention. In one aspect, said method at least comprises the steps of:
a) providing a set, collection or library of amino acid sequences; and
b) screening said set, collection or library of amino acid sequences for amino acid sequences that under the first biological condition can bind to said desired molecule with a k_{off} rate of 0.1 s⁻¹ and 10⁻⁶ s⁻¹, e.g. such a k_{off} as 0.01 to 0.00001; and
c) screening said set, collection or library of amino acid sequences for amino acid sequences that under said second biological condition bind to said desired molecule with a k_{off} rate that is at least 1.5 times or more than the k_{off} rate with which said amino acid sequence binds to said desired molecule under said first biological condition, more preferably the k_{off} rate is 1.7 times or more, more preferably the k_{off} rate is 2 times or more, more preferably the k_{off} rate is 3 times or more, more preferably the k_{off} rate is 4 times or more, more preferably the k_{off} rate is 5 times or more, more preferably the k_{off} rate is 10 times or more; and
d) isolating said amino acid sequence(s).

In another embodiment of the invention, such a method can comprise the steps of:
a) providing a set, collection or library of amino acid sequences; and
b) screening said set, collection or library of amino acid sequences for amino acid sequences that under the first biological condition can bind to said desired molecule with a k_{off} rate of 0.1 s⁻¹ and 10⁻⁶ s⁻¹, e.g. such a k_{off} as 0.01 to 0.00001; and
c) screening said set, collection or library of amino acid sequences for amino acid sequences that under said second biological condition bind to said desired molecule with a k_{off} rate that is at least 2 times or more than the k_{off} rate with which said amino acid sequence binds to said desired molecule under said first biological condition; and
d) isolating said amino acid sequence(s).

In another embodiment of the invention, such a method can comprise the steps of:
a) providing a set, collection or library of amino acid sequences; and
b) screening said set, collection or library of amino acid sequences for amino acid sequences that under the first biological condition, e.g. a pH between 7.2 to 7.4, e.g. 7.2, can bind to said desired molecule with a k_{off} rate of 0.1 s⁻¹ and 10⁻⁶ s⁻¹, e.g. such a k_{off} as 0.01 to 0.00001; and
c) screening said set, collection or library of amino acid sequences for amino acid sequences that under said second biological condition, e.g. a pH between 5 and 6, e.g. pH 5.5, bind to said desired molecule with a k_{off} rate that is at least 2 times or more than the k_{off} rate with which said amino acid sequence binds to said desired molecule under said first biological condition; and
d) isolating said amino acid sequence(s); and optionally
e) evaluate in vivo (e.g. PK evaluation in Cynomolgus monkey) the half life of said amino acid sequences.

In another embodiment of the invention, such a method can comprise the steps of:
a) providing a set, collection or library of amino acid sequences; and
b) screening said set, collection or library of amino acid sequences for amino acid sequences that under the first biological condition, e.g. pH between 7.2 to 7.4, e.g. 7.3, can bind to said desired molecule with a k_{off} rate of 0.1 s⁻¹ and 10⁻⁶ s⁻¹, e.g. such a k_{off} as 0.01 to 0.00001; and
c) (screening said set, collection or library of amino acid sequences for amino acid sequences that under said second biological condition, e.g. a pH between 5 and 6, e.g. pH 5.5, bind to said desired molecule with a k_{off} rate that is at least 2 times or more such as 3 times, 5 times, 10 times, 100 times, 1000 times than the k_{off} rate with which said amino acid sequence binds to said desired molecule under said first biological condition; or
d) screening said set, collection or library of amino acid sequences for amino acid sequences that under said second biological condition does not bind to said desired molecule), and
e) isolating said amino acid sequence(s); and optionally
f) evaluate in vivo (e.g. PK evaluation in Cynomolgus monkey) the half life of said amino acid sequences.

In another embodiment of the invention, such a method can comprise the steps of:
a) providing a set, collection or library of amino acid sequences; and
b) screening said set, collection or library of amino acid sequences for amino acid sequences that under the second biological condition, e.g. a pH between 5 and 6, e.g. pH 5.5, can bind to said desired molecule with a k_{off} rate of 0.1 and s⁻¹ and 10⁻⁶ s⁻¹, e.g. such a k_{off} as 0.01 to 0.00001; and
c) (screening said set, collection or library of amino acid sequences for amino acid sequences that under said first biological condition, e.g. a pH between 7.2 and 7.4, e.g. pH 7.3, bind to said desired molecule with a k_{off} rate that is at least 2 times or more such as 3 times, 5 times, 10 times, 100 times, 1000 times than the k_{off} rate with which said amino acid sequence binds to said desired molecule under said second biological condition; or
d) screening said set, collection or library of amino acid sequences for amino acid sequences that under said first biological condition does not bind to said desired molecule), and
e) isolating said amino acid sequence(s); and optionally
f) evaluate in vivo (e.g. PK evaluation in Cynomolgus monkey) the half life of said amino acid sequences.

As will be clear to the skilled person, the screening step can also be performed as a selection step. For example, antibody-antigen interactions are known to be often sensitive to changes in buffer conditions, pH and ionic strength, but most often those changes are not scored or investigated, and they are not often used to design drug therapeutics as variations are overall unpredictable. Binding proteins with the desirable binding characteristics are found for example by screening repertoires of binding proteins for the occurrence of a sensitive interaction, e.g. by carrying out a binding assay with under the first and second biological condition, respectively, and the relative binding strength determined. Such strength of relative interaction can be measured with any suitable binding test including ELISA, BIAcore-based methods, Scatchard analysis etc. Such test will reveal which binding proteins display interactions that are sensitive to the chosen parameter (pH) and to what extent. Binding proteins with the desirable binding characteristics are alternatively found by selecting repertoires of binding proteins, e.g. from phage, ribosome, yeast or cellular libraries using conditions in the selection that will preferentially enrich for the desirable sensitivity. Taking a first and second biological condition that differ in respect of pH as an example, incubating a phage antibody library at basic pH (e.g. pH 7.4) and eluting the bound phage particles with a buffer of lower pH (e.g. 6.0) will enrich for those phage antibodies that are recognizing antigen sensitive to this pH change. A repetitive cycle of such selections is then followed by screening of individual clones to identify the binding protein that displays pH-dependent binding in this pH window. Binding proteins with the desirable binding characteristics can further be isolated from designer protein libraries in which the putative binding site has been engineered to contain amino acid residues or sequences that are preferred in certain ‘sensitive’ interactions, e.g. histidines for pH-sensitivity. For example, it is known that the interaction between FcRn and IgG is exquisitely sensitive to pH, being reduced over 2 orders of magnitude as the pH is raised from pH 6.0 to 7.0. The main mechanistic basis of the affinity transition is the histidine content of the binding site: the imidazole side changes of histidine residues usually deprotonate over the pH range 6.0-7.0. The explicit inclusion of histidines in the putative binding site (e.g. using oligonucleotides that preferentially introduce this residue in the library, as with the use of trinucleotides and known in the field. e.g. Knappik et al, J. Mol. Biol. 2000, vol 296:57-86) is predicted to yield a higher frequency of amino acid sequences that bind essentially dependent of the pH.

Accordingly the term “screening” as used in the present description can comprise selection, screening or any suitable combination of selection and/or screening techniques.

In general, steps b) and c) can be performed as single or separate screening steps, or as part of a single screening process. When steps b) and c) are performed as part of a single screening process, such a screening process may for example comprise the steps of:
i) bringing the set, collection or library of amino acid sequences in contact with the desired molecule under the first biological condition (i.e. such that amino acid sequences that can bind to said desired molecule with a dissociation constant (K_{D}) of 10⁻⁵ moles/liter or less bind to the desired molecule, and such that amino acid sequences that can not bind to said desired molecule with a dissociation constant (K_{D}) of 10⁻⁵ moles/liter or less do not bind to the desired molecule);
ii) removing the amino acid sequence that do not bind in step i) (i.e. those amino acid sequences that under the first biological condition do not bind to said desired molecule with a dissociation constant (K_{D}) of 10⁻⁵ moles/liter or less); so that a set or collection of amino acid sequences remains that is bound to the intended to desired molecule (and that is still present under the first biological condition);
iii) subjecting the set or collection of amino acid sequences to the second biological condition, such that amino acid sequences that do not conditionally hind (as defined herein) to the intended or desired molecule stay bound to the intended or desired molecule, and such that amino acid sequences that conditionally bind (as defined herein) to the intended or desired molecule no longer stay bound to the intended or desired molecule;
iv) separating the amino acid sequences that conditionally bind (as defined herein) to the intended or desired molecule from the amino acid sequences that do not conditionally bind (that are still bound to the desired molecule);
   and optionally
v) collecting the amino acid sequences that conditionally bind to the intended or desired molecule.

For example, this single screening process can easily be performed by providing a suitable carrier or support (such as a column, beads, or solid surface such as the surface of a well of a multi-well plate, or the stationary phase of a Biacore) onto which the desired molecule is suitably immobilized (for example covalently or via an avidin-steptavidin linkage); contacting the carrier or support with the set, collection or library of amino acid sequences; washing away the amino acid sequences that do not bind to the desired molecule bound to the carrier or support; changing the conditions to the second biological condition, and collecting the amino acid sequences that under the second biological condition do not bind to the to the desired molecule bound to the carrier or support.

Alternatively, as described in more detail below, amino acid sequences of the invention may be obtained by enriching a set, collection or library of amino acid sequences (as described herein) for conditional binders that bind to the desired molecule,

The set, collection or library of amino acid sequences used in the above method(s) can be any suitable set, collection or library of amino acid sequences. For example, the set, collection or library of amino acid sequences may be a set, collection or library of immunoglobulin sequences or of fragments of immunoglobulin sequences, such as a set, collection or library of immunoglobulin variable domain sequences or a fragments thereof, e.g. a set, collection or library of V_{H}-, V_{L}- or V_{HH}-sequences or a fragments thereof. In one specific, but non-limiting aspect, the set, collection or library of amino acid sequences a set, collection or library of domain antibodies, of proteins that can be used as domain antibodies, of “dAb”, of single domain antibodies, of proteins that can be used as single domain antibodies, or of Nanobodies (or of suitable fragments of any of the foregoing).

The set, collection or library of amino acid sequences may be a naïve set, collection or library; may be a set, collection or library of synthetic or semi-synthetic amino acid sequences (for example, without limitation, a set, collection or library of amino acid sequences that has been generated by affinity maturation), or may be an immune set, collection or library. In one embodiment, the set, collection or library is an immune set, collection or library that has been obtained by suitably immunizing a mammal (such as a rabbit, rat, mouse, pig or dog, or Camelid) with an antigen (such that said mammal forms antibodies against said antigen), and then generating a set, collection or library of immunoglobulin sequences starting from a biological sample (such as blood or a sample of B-cells) obtained from said mammal. Methods and techniques for obtaining and screening such an immune set, collection or library will be clear to the skilled person, for example from the prior art cited herein. In one preferred aspect, the set, collection or library of immunoglobulin sequences is obtained from a mammal that has been suitably immunized with the intended serum protein (e.g. with serum albumin). In another preferred aspect, the set, collection or library is a set, collection or library of V_{HH} sequences obtained from a Camelid, and in particular an immune set, collection or library of V_{HH} sequences obtained from a Camelid that has been suitably immunized with the intended serum protein (e.g. with serum albumin).

The set, collection or library may contain any suitable number of amino acid sequences, such as 1, 2, 3 or about 5, 10, 50, 100, 500, 1000, 5000, 10⁴, 10⁵, 10⁶, 10⁷, 10⁸ or more sequences.

The above set, collection or library of amino acid sequences may contain one or more sequences that are not known in advance of the selection and/or screening process for example if these sequences are the result of a randomization step (e.g. via error-prone PCR or other means) of one or more given amino acid sequences. Also, one or more or all of the amino acid sequences in the above set, collection or library of amino acid sequences may be obtained or defined by rational, or semi-empirical approaches such as computer modelling techniques or biostatics or data-mining techniques wherein amino acid sequences may have been defined or proposed that are predicted or expected to be endowed with certain properties such as increased stability, pH optimum, protease sensitivity or other properties or combinations thereof.

In such a set, collection or library (and/or during the screening steps described herein), the amino acid sequences present in said set, collection or library may also be suitably displayed on a suitable host or host cell, for example on phage particles, ribosomes, bacteria, yeast cells, etc. Again, suitable hosts or host cells, suitable techniques for displaying amino acid sequences on such hosts or host cells, and suitable techniques for screening a set, collection or library of amino acid sequences displayed on such hosts or host cells will be clear to the skilled person, for example from the prior art cited herein. When the amino acid sequence(s) are displayed on a suitable host or host cell, it is also possible (and customary) to first isolate from said host or host cell a nucleotide sequence that encodes the desired amino acid sequence, and then to obtain the desired amino acid sequence by suitably expressing said nucleotide sequence in a suitable host organism. Again, this can be performed in any suitable manner known per se, as will be clear to the skilled person.

By means of non-limiting example, such set, collection or library can comprise one, two or more amino acid sequences that are variants from one another (e.g. with designed point mutations or with randomized positions), compromise multiple amino acid sequences derived from a diverse set of naturally diversified amino acid sequences (e.g. an immune library)), or any other source of diverse amino acid sequences (as described for example in Hoogenboom et al, Nat Biotechnol 23:1105, 2005 and Binz et al, Nat Biotechnol 2005, 23:1247). Such set, collection or library of amino acid sequences can be displayed on the surface of a phage particle, a ribosome, a bacterium, a yeast cell, a mammalian cell, and linked to the nucleotide sequence encoding the amino acid sequence within these carriers. This makes such set, collection or library amenable to selection procedures to isolate the desired amino acid sequences of the invention.

The amino acid sequences of the invention may also contain one or more additional binding sites for one or more other antigens, antigenic determinants, proteins, polypeptides, or other compounds.

The amino acid sequences disclosed herein can be used with advantage as a fusion partner for other moieties (such as other amino acid sequences, proteins or polypeptides, or other chemical entities), and in particular as a fusion partner for therapeutic moieties such as therapeutic proteins or polypeptides, therapeutic compounds (including, without limitation, small molecules) or other therapeutic entities. Such a construct or fusion comprising at least one amino acid sequence of the invention and at least one further compound, moiety or entity is also referred to herein as a “compound of the invention”.

Thus, in another aspect, the invention provides compounds such as polypeptide or protein constructs that comprise or essentially consist of an amino acid sequence as disclosed herein that is linked to at least one therapeutic moiety, optionally via one or more suitable linkers or spacers. Such polypeptide or protein construct may for example (without limitation) be a fusion protein, as further described herein.

The invention further relates to therapeutic uses of polypeptide or protein constructs or fusion proteins and to pharmaceutical compositions comprising such polypeptide or protein constructs or fusion proteins.

In some embodiments the at least one therapeutic moiety comprises or essentially consists of a therapeutic protein, polypeptide, compound, factor or other entity. In a preferred embodiment the therapeutic moiety is directed against a desired antigen or target, is capable of binding to a desired antigen (and in particular capable of specifically binding to a desired antigen), and/or is capable of interacting with a desired target. In another embodiment, the at least one therapeutic moiety comprises or essentially consists of a therapeutic protein or polypeptide. In a further embodiment, the at least one therapeutic moiety comprises or essentially consists of an immunoglobulin or immunoglobulin sequence (including but not limited to a fragment of an immunoglobulin), such as an antibody or an antibody fragment (including but not limited to an ScFv fragment). In yet another embodiment, the at least one therapeutic moiety comprises or essentially consists of an antibody variable domain, such as a heavy chain variable domain or a light chain variable domain.

In a preferred embodiment, the at least one therapeutic moiety comprises or essentially consists of at least one domain antibody or single domain antibody, “dAb” or Nanobody®, so that the resulting polypeptide or protein construct or fusion protein is a multivalent construct and preferably a multispecific construct.

By a “multivalent” compound, protein, polypeptide or construct is meant in this description a compound, protein, polypeptide or construct that comprises at least two binding units (i.e. binding to the same or different epitopes), all of which can bind to the same (type of) biological molecule. By a “bivalent” compound, protein, polypeptide or construct is meant in this description, a compound, protein, polypeptide or construct that comprises two binding units, which can bind to the same (type of) biological molecule. By a “monovalent” compound, protein or polypeptide is meant in this description, a compound, protein or polypeptide that essentially consists of one binding unit, which can bind to a biological molecule.

By “binding unit” is meant in this description any amino acid sequence, peptide, protein, polypeptide, construct, fusion protein, compound, factor or other entity capable of binding a biological molecule as described herein, such as an amino acid sequence of the invention or a therapeutic moiety (both as described herein). When a compound, protein, polypeptide or construct comprises two or more binding units, said binding units may optionally be linked to each other via one or more suitable linkers.

By a “multispecific” compound, protein, polypeptide or construct is meant in this description, a compound, protein, polypeptide or construct that comprises at least two binding units, of which at least a first binding unit can bind to a first biologically functional molecule and of which at least a second binding unit can bind to a second biologically functional molecule. By a “bispecific” compound, protein, polypeptide or construct is meant in this description, a compound, protein, polypeptide or construct that comprises two binding unit, of which the first binding unit can bind to a first biologically functional molecule and of which the second binding unit can bind to a second biologically functional molecule. The first and second biologically functional molecule may be different molecules or may be the same biological molecule in which case the bispecific compound recognizes or binds to the biological molecule at to different sites.

In a specific embodiment, the at least one therapeutic moiety comprises or essentially consists of at least one monovalent Nanobody® or a bivalent, multivalent, bispecific or multispecific Nanobody® construct.

In another specific embodiment, the compounds of the invention may comprise two or more amino acid sequences of the invention (and optionally one or more further moieties as described herein), optionally linked via one or more suitable linkers, in which the two or more amino acid sequences of the invention may be directed against the same desired or intended molecule (for example, to provide a conditional binder with increased avidity under the first biological condition), against different parts of epitopes on the same desired or intended molecule (again, for example, to provide a conditional binder with increased avidity under the first biological condition), or against different intended or desired molecules.

According to this last non-limiting embodiment, a compound of the invention may be a bispecific (or multispecific) compound that conditionally binds to two or more different intended desired molecules. As such, the compound of the invention may be such that it binds to both a first and a second intended or desired molecule under the first biological condition (or alternatively, under the second biological condition), or may be such that it binds to a first intended or desired molecule under the first biological condition but binds to a second intended or desired molecule under the second biological condition. Thus, when changing from the first biological condition to the second biological condition, such a compound of the invention will therefore be released from the first intended or desired molecule and bind to the second intended or desired molecule.

Some specific but non-limiting applications of conditional binders in such compounds of the invention for the purposes of extending the half-life of therapeutic compounds, moieties or entities will become clear from the further description herein,

In other embodiments, a compound of the invention may comprises at least one conditional binding unit as described herein, and one or more further binding units that are not conditional binders.

Again, some specific but non-limiting applications of conditional binders in such compounds of the invention for the purposes of extending the half-life of therapeutic compounds, moieties or entities will become clear from the further description herein.

The invention also relates to nucleotide sequences or nucleic acids that encode amino acid sequences, compounds, proteins, polypeptides, fusion proteins, or multivalent or multispecific constructs described herein. The invention further includes genetic constructs that include the foregoing nucleotide sequences or nucleic acids and one or more elements for genetic constructs known per se. The genetic construct may be in the form of a plasmid or vector. Such and other genetic constructs are known by those skilled in the art.

The invention also relates to hosts or host cells that contain such nucleotide sequences or nucleic acids, and/or that express (or are capable of expressing) amino acid sequences, compounds, proteins, polypeptides, fusion proteins, or multivalent or multispecific constructs described herein. Again, such hosts or host cells are known by those skilled in the art.

The invention also generally relates to a method for preparing amino acid sequences, compounds, proteins, polypeptides, fusion proteins, or multivalent or multispecific constructs as described herein, which method comprises cultivating or maintaining a host cell as described herein under conditions such that said host cell produces or expresses an amino acid sequence, compound, protein, polypeptide, fusion protein, or multivalent or multispecific construct as described herein, and optionally further comprises isolating the amino acid sequence, compound, protein, polypeptide, fusion protein, or multivalent or multispecific construct so produced. Again, such methods can be performed as generally described in the co-pending patent applications by applicant mentioned herein.

The amino acid sequences and compounds of the invention can be designed and used for any suitable purpose known per se, depending on the choice of the intended or desired compound(s) against which the conditional binder(s) present in the compound of the invention is or are directed, and also dependent on the further moieties, compounds or binding units (that may be either conditional or non-conditional binding units) that are present in the compound of the invention. Such purposes and amino acid sequences and compounds of the invention suitable for such applications will be clear to the skilled person based on the disclosure herein.

According to one specific, but non-limiting application of the invention, the amino acid sequences of the invention are directed against a serum protein, and can be used as a fusion partner, binding unit or moiety for increasing the half-life of a therapeutic moiety or compound (as described herein).

Amino acid sequences that are capable of binding to serum proteins and uses thereof in polypeptide constructs in order to increase the half-life of therapeutically relevant proteins, polypeptides and other compounds are known in the art.

For example, WO 91/01743, WO 01/45746 and WO 02/076489 describe peptide moieties binding to serum albumin that can be fused to therapeutic proteins and other therapeutic compounds and entities in order to increase the half-life thereof. However, these peptide moieties are of bacterial or synthetic origin, which is less preferred for use in therapeutics.

The neonatal Fc receptor (FcRn), also termed “Brambell receptor”, is involved in prolonging the life-span of albumin in circulation (see Chaudhury et al., The Journal of Experimental Medicine, vol. 3, no. 197, 315-322 (2003)). The FcRn receptor is an integral membrane glycoprotein consisting of a soluble light chain consisting of β2-microglobulin, noncovalently bound to a 43 kD α chain with three extracellular domains, a transmembrane region and a cytoplasmic tail of about 50 amino acids. The cytoplasmic tail contains a dinucleotide motif-based endocytosis signal implicated in the internalization of the receptor. The α chain is a member of the nonclassical MHC I family of proteins. The β2m association with the cc chain is critical for correct folding of FcRn and exiting the endoplasmic reticulum for routing to endosomes and the cell surface.

The overall structure of FcRn is similar to that of class 1 molecules. The α-1 and α-2 regions resemble a platform composed of eight antiparallel β strands forming a single β-sheet topped by two antiparallel α-helices very closely resembling the peptide cleft in MHC I molecules. Owing to an overall repositioning of the α-1 helix and bending of the C-terminal portion of the α-2 helix due to a break in the helix introduced by the presence of Pro162, the FcRn helices are considerably closer together, occluding peptide binding. The side chain of Arg164 of FcRn also occludes the potential interaction of the peptide N-terminus with the MHC pocket. Further, salt bridge and hydrophobic interaction between the α-1 and α-2 helices may also contribute to the groove closure.

FcRn therefore, does not participate in antigen presentation, and the peptide cleft is empty.

FcRn binds and transports IgG across the placental syncytiotrophoblast from maternal circulation to fetal circulation and protects IgG from degradation in adults. In addition to homeostasis, FcRn controls transcytosis of IgG in tissues. FcRn is localized in epithelial cells, endothelial cells and hepatocytes.

According to Chaudhury et al. (supra), albumin binds FcRn to form a tri-molecular complex with IgG. Both albumin and IgG bind noncooperatively to distinct sites on FcRn. Binding of human FcRn to Sepharose-HSA and Sepharose-hIgG was pH dependent, being maximal at pH 5.0 and nil at pH 7.0 through pH 8. The observation that FcRn binds albumin in the same pH dependent fashion as it binds IgG suggests that the mechanism by which albumin interacts with FcRn and thus is protected from degradation is identical to that of IgG, and mediated via a similarly pH-sensitive interaction with FcRn. Using SPR to measure the capacity of individual HSA domains to bind immobilized soluble hFcRn, Chaudhury showed that FcRn and albumin interact via the D-III domain of albumin in a pH-dependent manner, on a site distinct from the IgG binding site (Chaudhury, PhD dissertation, see http://www.andersonlab.com/biosketchCC.htm; Chaudhury et al. Biochemistry, ASAP Article 10.1021/bi052628y S0006-2960(05)02628-0 (Web release date: Mar. 22, 2006)).

WO 04/041865 by applicant describes Nanobodies® capable of binding to serum albumin (and in particular against human serum albumin) that can be linked to other proteins (such as one or more other Nanobodies® capable of binding to a desired target) in order to increase the half-life of said protein. It is known that these Nanobodies® are more potent and more stable than conventional four-chain serum albumin binding antibodies which leads to (1) lower dosage forms, less frequent dosage leading to less side effects; (2) improved stability leading to a broader choice of administration routes, comprising oral or subcutaneous routes in addition to the intravenous route; (3) lower treatment cost due to lower cost of goods.

In this embodiment of the invention, the desired molecule is a serum protein, and in particular a serum protein that is subjected to recycling within the human or animal body. Some non-limiting examples of such serum proteins are serum proteins that can bind to FcRn such as serum albumin and IgG. Other serum proteins to which the amino acid sequences of the invention can bind will be clear to the skilled person, and for example include the serum proteins mentioned in the International application WO 04/003019 (see also EP 1 517 921).

Thus, according to this embodiment, the invention relates to an amino acid sequence that is directed against a serum protein, wherein said amino acid sequence:
a) binds to said serum protein under a first biological condition with a dissociation constant (K_{D}) of 10⁻⁵ moles/liter or less and/or with a binding affinity (K_{A}) of at least 10⁵ M⁻¹; and
b) binds to said serum protein under a second biological condition with a dissociation constant (K_{D}) that is at least 10 fold more than the dissociation constant with which said amino acid sequence binds to said serum protein under said first biological condition.

The serum protein to which the amino acid sequences of the invention bind (or under physiological conditions can bind) may be any serum protein (such as those mentioned herein and in WO 04/003019, and may in particular be any serum protein that is subject to recycling or a recycling mechanism in the human or animal body in which said serum protein naturally occurs. Examples of such serum proteins will be clear to the skilled person.

More in particular, the serum protein to which the amino acid sequences of the invention hind (or under physiological conditions can bind) may be chosen from the group consisting of: serum albumin, immunoglobulins such as IgG and transferrin. According to a preferred, but non-limiting embodiment, the amino acid sequences of the invention bind to serum albumin.

The serum protein is preferably a human serum protein, such as human serum albumin, IgG or transferrin, and in particular human serum albumin. However, it should be understood that according to some specific but non-limiting aspects of the invention, the amino acid sequences of the invention may be cross-reactive with the corresponding (i.e. orthologous) serum protein from at least another species of mammal, such as mouse, rat, rabbit, dog or primate. In particular, according to these aspects, the amino acid sequences of the invention may be cross-reactive with the corresponding (i.e. orthologous) serum protein from at least another species of primate, as further described herein.

In particular, according to this embodiment, the amino acid sequence of the invention may bind to said serum protein under said second biological condition with a dissociation constant (K_{D}) that is at least 10-fold more, preferably 100 fold more, more preferably 1000 fold more, than the dissociation constant with which said amino acid sequence binds to said serum protein under said first biological condition. In a preferred embodiment, the amino acid sequence of the invention, e.g. a single chain antibody, e.g. a dAb or a Nanobody, does not bind at all under said second biological condition, e.g. the amino acid sequence of the invention does not bind at pH5.5 (or at a pH between 5 to 6) but binds at physiological pH, i.e. pH 7.2 to 7.4.

Preferably, according to this embodiment, the amino acid sequence of the invention binds to said serum protein under said first biological condition with a dissociation constant (K_{D}) of 10⁻⁶ moles/liter or less, preferably with a dissociation constant (K_{D}) of 10⁻⁷ moles/liter or less, more preferably with a dissociation constant (K_{D}) of 10⁻⁸ moles/liter or less.

Also, preferably, according to this embodiment, the amino acid sequence of the invention binds to said serum protein under said second biological condition with a dissociation constant (K_{D}) of 10⁻⁶ moles/liter or more, preferably with a dissociation constant (K_{D}) of 10⁻⁵ moles/liter or more, more preferably with a dissociation constant (K_{D}) of 10⁻⁴ moles/liter or more.

In another embodiment of this invention, the amino acid sequence of the invention binds to said serum protein under said second biological condition with a dissociation constant (K_{D}) that is at least 10-fold less, 100 fold less, preferably 1000 fold less, than the dissociation constant with which said amino acid sequence binds to said serum protein under said first biological condition.

In another embodiment of this invention, the amino acid sequence of the invention binds to said serum protein under said first biological condition with a dissociation constant (K_{D}) of 10⁻⁶ moles/liter or more, and under said second biological condition with a dissociation constant (K_{D}) of 10⁻⁷ moles/liter or less, e.g. 10⁻⁸ moles/liter or less or 10⁻⁹ moles/liter or less.

In another embodiment of this invention, the amino acid sequence of the invention binds to said serum protein under said first biological condition with a dissociation constant (K_{D}) of 10⁻⁵ moles/liter or more, and under said second biological condition with a dissociation constant (K_{D}) of 10⁻⁶ moles/liter or less, e.g. 10⁻⁷ moles/liter or less; or e.g. 10⁻⁸ moles/liter or less.

In another embodiment of this invention, the amino acid sequence of the invention binds to said serum protein under said first biological condition with a dissociation constant (K_{D}) of 10⁻⁴ moles/liter or more, and under said second biological condition with a dissociation constant (K_{D}) of 10⁻⁵ moles/liter or less, e.g. 10⁻⁶ moles/liter or less; or e.g. 10⁻⁷ moles/liter or less.

In a preferred embodiment, the amino acid sequence of the invention, e.g. a single chain antibody, e.g. a dAb or a Nanobody, does not bind at all under said first biological condition, e.g. the amino acid sequence of the invention binds at endosomal pH5.5 (or at a pH between 5 to 6) but does not bind at extracellular pH, i.e. at pH 7.2 to 7.4.

In a further preferred embodiment, the amino acid sequence of the invention, e.g. a single chain antibody, e.g. a dAb or a Nanobody, is a bivalent or multivalent amino acid sequence, wherein one binding block is directed against human serum albumin and wherein said human serum albumin binding block does not bind at pH 5.5 (or at a pH between 5 to 6) but binds at pH 7.2 to 7.4; and optionally the amino acid sequence of the invention, e.g. a single chain antibody, e.g. a dAb or a Nanobody, is binding to a target protein for therapeutic intervention (e.g. in a monovalent or multivalent, e.g. bivalent format).

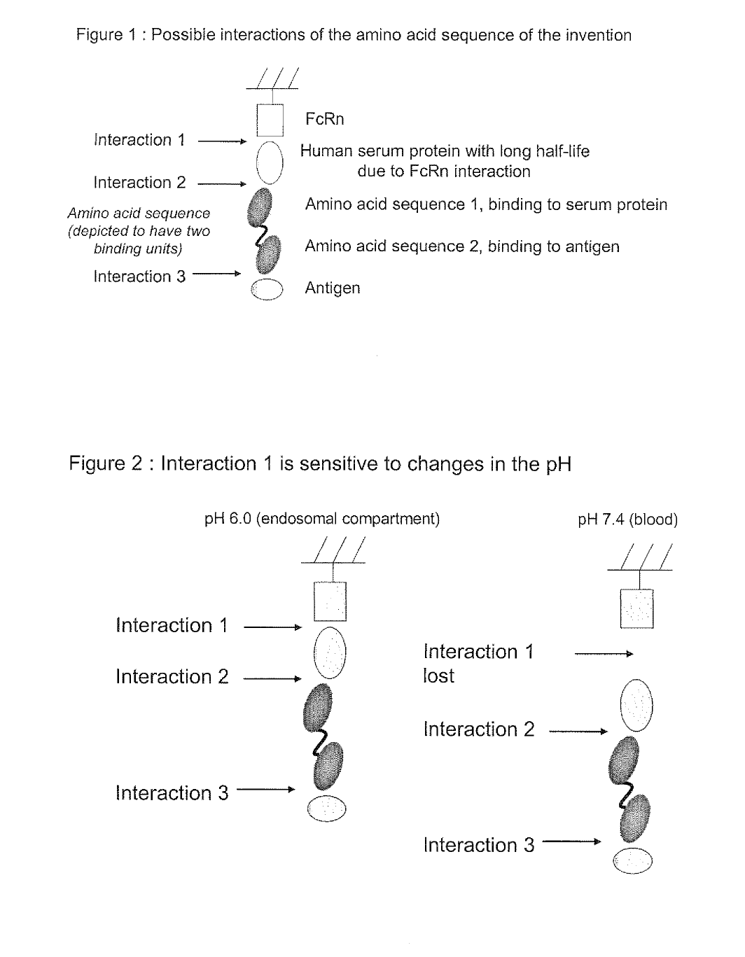

An example of such targets for therapeutic intervention are proteins of the TNF superfamily (Aggarwal, Nature Reviews Immunology 3: 747, 2003). This superfamily of proteins consists of 19 members that signal through 29 receptors. These ligands, while regulating normal functions such as immune responses, haematopoiesis and morphogenesis, have also been implicated in tumorgenesis, transplant rejection, septic shock, viral replication, bone resorption, rheumatoid arthritis and diabetes. Blockers of TNF have been approved for human use in treating TNF-linked autoimmune diseases. Whereas most ligands bind to a single receptor, others bind to more than one. For example, TRAIL binds to as many as five receptors (DR4, DR5, DVR1, DCR2 and OPG), whereas BAFF binds to three receptors, transmembrane activator and cyclophilin ligand interactor (TACI), B-cell maturation antigen (BMCA) and BAFFR (Aggarwal, 2003, FIG. 1). There is also evidence for crosstalk between receptors for different ligands of the TNF superfamily. It follows that, in order to achieve maximal therapeutic benefit, the interactions of all ligands with a particular receptor, or the interactions of a particular ligand with all its receptors should be inhibited at the same time. Therefore, for efficient therapy, various different binding molecules or binding molecules with multiple binding specificity are required.

Another example of possible targets for therapeutic intervention is a sub-family of the Receptor Tyrosin Kinases, the Eph family, comprised of 16 known Eph receptors (14 found in mammals) and 9 known ephrin ligands (8 found in mammals). The ability of the Eph receptor and ephrin ligand guidance system to position cells and modulate cell morphology reflects their various roles in development. These membrane anchored ligands and receptors are involved in bi-directional signaling (into both the receptor bearing cell and the ligand bearing cell. Eph receptors, first shown to be important regulators of axon path-finding and neuronal cell migration (Drescher et al., Cell 82: 359, 1995; Henkemeyer et al., Cell 86: 35, 1996), are now known to have roles in controlling a diverse array of other cell-cell interactions, including those of vascular endothelial cells (Wang et al., Cell 93: 741, 1998; Adams et al., Genes Dev. 13: 295, 1999; Gerety et al., Mol. Cell. 4: 403, 1999) and specialized epithelia (Orioli et al., EMBO J. 15: 6035, 1996; Flanagan and Vanderhaeghen Annu. Rev. Neurosci. 21: 309, 1998; Frisen et al, EMBO J. 18: 5159, 1999; Cowan et al., Neuron 26: 417, 2000). Ephrins and the ephrin receptor bidirectional signaling have been implicated in axonal guidance, angiogenesis and bone remodeling. Therapeutically, there is interest in antagonizing certain ephrin-Eph receptor signaling processes.

The ephrins and the Eph receptors are divided into two classes A and B based on their affinities for each other and sequence conservation. In general, the nine different EphA RTKs (EphA1-EphA9) bind promiscuously to, and are activated by, six A-ephrins (ephrinA1-ephrinA6), and the EphB subclass receptors (EphB1-EphB6 and, in some cases, EphA4) interact with three different B-ephrins (ephrinB1-ephrinB3). In order to achieve maximal therapeutic benefit, therefore, interactions of all ephrin ligands with a particular Eph receptor, or the interactions of a particular ephrin with all its Eph receptors should be inhibited at the same time. Accordingly, also here, for efficient therapy, various different binding molecules or binding molecules with multiple binding specificity will be needed.

The costimulatory molecules of the B7 superfamily are another example of possible targets for therapeutic intervention. The presence of co-stimulatory molecules on the APC is required (“signal 2”) alongside antigenic peptide in the context of the MHC molecule (“signal 1”) to obtain efficient stimulation of naïve antigen reactive T-cells. CD80, CD86, CD28, cytotoxic T lymphocyte antigen 4 (CTLA4), inducible costimulator (ICOS), programmed death 1 (PD-1), and OX 40 are used as targets to manipulate T-cells to slow the progression of autoimmune diseases, or to treat tumors through the increase in T-cell activation. CD80 (previously called B7-1) and CD86 (B7-2) are expressed on the membrane of activated antigen presenting cells (APC) such as dendritic cells, macrophages or B-cells. The presence of costimulatory molecules is sensed by counterreceptors on the surface of the T-cell. Selective blockade of the interaction of such costimulatory molecules with their cognate activating receptor (CD28) on the T-cell may therefore inhibit T-cell activation (Howard et al., Curr. Drug Targets Inflamm. Allergy 4: 85, 2005; Stuart and Racke, Expert Opinion Ther. Targets 6: 275, 2002).

Activated self-antigen directed T-cells are responsible for at least part of the tissue damage in autoimmune diseases such as rheumatoid arthritis or multiple sclerosis by virtue of their effector function, and indirectly for production of high-affinity self-reactive antibodies by providing “help” to B-cells. Thus, blockade of the interaction of CD80 and/or CD86 with CD28 can be therapeutic in autoimmune conditions. These principles have been firmly established in both animal models of human disease, as well as in man, by using either blocking monoclonal antibodies directed against CD80 or CD86, or using soluble forms of a counterreceptor (Stuart and Racke, 2002).

CD152 (previously known as CTLA4) is another counterreceptor on T-cells for both CD80 and CD86. Unlike CD28, however, interaction of CD152 with CD80 and/or CD86 does not lead to T-cell activation. CD152 is thought to interact with both CD80 and CD86 with a higher affinity than CD28, and may therefore serve as a decoy receptor for CD28, depriving the latter of its ligands and therefore indirectly decreasing T-cell activation (Collins et al., Immunity 17: 201, 2002). Alternatively, CD152 may also transduce a negative signal into the T-cell, leading to lower overall levels of T-cell activation. Regardless of the mechanism, the activity of CD152 signaling leads to a dampening of T-cell responses, especially late (48-72H) after T-cell stimulation when surface CD152 expression becomes high. Blocking CD152 signaling by the use of monoclonal antibodies blocking its interaction with CD80 and/or CD86 increases the level of T-cell activation in vivo, and this has been demonstrated to be beneficial as an adjunct treatment in tumor vaccine therapies. Since inhibition of CTLA4 signaling leads to very different outcomes than CD28 blockade during T-cell activation, it may be beneficial to design a CD80 and/or CD86 neutralizing therapeutic entity which inhibits the interaction of CD80 and/or CD86 with CD28 but not CTLA4, or vice versa.

CD80 and CD86 are also present at high levels on many lymphomas of B-cell origin. Thus, monoclonal antibodies, fragments thereof and other proteins binding CD80 and/or CD86 can be useful in the therapy of such tumors, either by recruiting effector functions, induction of cell death or as a targeting entity in immunotoxins or radiotoxin conjugates (Friedberg et al., Blood 106: 11 Abs 2435, 2005).

As both CD80 and CD86 bind to either counterreceptor, these molecules are thought to have at least partially overlapping functional roles (partial functional redundancy). It follows that, in order to achieve maximal therapeutic benefit, interactions of both CD80 and CD86 with either CD28 or CD152 need to be inhibited at the same time. Potentially, this can be achieved using soluble forms of CD152 (Abatacept, CTLA4-Ig, see Linsley et al. J. Exp. Med. 174: 561, 1991), affinity variants thereof (Belatacept, LEA29Y, see Larsen et al., Am. J. Transplant 5: 443, 2005) or CD28 (CD28-Ig, see Linsley et al., J. Exp. Med. 173: 721, 1991). No single monoclonal antibody has yet been described which can bind to both CD80 and CD86 (WO 04/076488, van den Beucken et al., J. Mol. Biol. 310: 591, 2001), although this would clearly be beneficial.

In a further preferred embodiment, the amino acid sequence of the invention, e.g. a single chain antibody, e.g. a dAb or a Nanobody, is a bivalent or multivalent amino acid sequence, wherein at least one binding block is directed against a serum albumin, e.g. human serum albumin, and wherein said serum albumin binding block binds at e.g. pH 5.5 (or e.g. at a pH between 5 to 6, or e.g. a ph 5.3 to 5.7) but does not bind at pH 7.2 to 7.4.

As described herein for the amino acid sequences of the invention, said first biological condition may comprise the physiological conditions prevalent in a first physiological compartment or fluid, and said second biological condition comprises the physiological conditions prevalent in a second physiological compartment or fluid, wherein the first and second physiological compartments are, under normal physiological conditions, separated by at least one biological membrane such as a cell membrane, a wall of a cellular vesicle or a subcellular compartment, or a wall of a blood vessel.

In particular, said first biological condition comprises the physiological conditions prevalent outside at least one cell of a human or animal body (such as the physiological conditions prevalent in the bloodstream or lymphatic system of said human or animal body), and said second biological condition comprises the conditions prevalent inside said cell (or vise versa, although this may be less preferred for the purposes of half-life extension).

For the purposes of this embodiment, by “the physiological conditions that are prevalent inside a cell of an animal or human body” is meant the conditions (such as the pH value(s)) that may occur inside a cell, and in particular inside a cell that is involved in the recycling of the serum protein. In particular, by “the physiological conditions that are prevalent inside a cell of an animal or human body” is meant the conditions (such as the pH value(s)) that may occur inside a (sub)cellular compartment or vesicle that is involved in recycling of the serum protein (e.g. as a result of pinocytosis, endocytosis, transcytosis, exocytosis and phagocytosis or a similar mechanism of uptake or internalization into said cell), such as an endosome, lysosome or pinosome.

For example, the cell may be a cell that contains or expresses the FcRn receptor, in particular when the amino acid sequence of the invention is directed against a serum protein that binds to FcRn. As will become clear from the further description herein, such cells are involved in recycling of certain serum proteins that can bind to FcRn, such as serum albumin and immunoglobulins such as IgG. Alternatively, for example and without limitation, the cell may be a cell that contains or expresses the transferrin-receptor, in particular when the amino acid sequence of the invention is directed against transferrin

For the purposes of this embodiment, by “the physiological conditions that are prevalent outside a cell of an animal or human body” is generally meant the conditions (such as the pH value(s)) that may occur inside the body of the human or animal in which said cell is present, but outside said cell, such as at the cell surface or in the immediate surroundings or near vicinity of said cell. In particular, by “the physiological conditions that are prevalent outside a cell of an animal or human body” is meant the conditions (such as the pH value(s)) that may occur in the circulation of the human or animal body in which said cell is present, such as in the blood(stream) or in the lymphatic system.

Thus, generally, in this embodiment, where the serum protein can be taken up (for example by internalization, pinocytosis, endocytosis, transcytosis, exocytosis, phagocytosis or a similar mechanism of uptake or internalization into said cell) by at least one cell of the human or animal body, wherein said first biological condition may comprise the physiological conditions in which the amino acid sequence is present prior to being taken up into the cell and the second biological condition may comprise the physiological conditions in which the amino acid sequence is present after being taken up into the cell. In particular, where the amino acid sequence of the invention is directed against a serum protein that is subject to recycling, wherein the first biological condition comprises the extracellular conditions (e.g. the conditions that are prevalent in the circulation) with respect to at least one cell of the animal or human body that is involved in recycling of the desired compound, and wherein the second biological condition comprises the conditions that are prevalent inside the at least one cell of the animal or human body that is involved in recycling of the desired compound.

According to another non-limiting aspect of this embodiment, the first biological condition may be a physiological pH of more than 7.0, and the second biological condition may be a physiological pH of less than 7.0. In particular, the first biological condition may be a physiological pH of more than 7.1, and said second biological condition may be a physiological pH of less than 6.7. More in particular, the first biological condition may be a physiological pH of more than 7.2, and the second biological condition may be a physiological pH of less than 6.5. More in particular, the first biological condition may be a physiological pH of more than 7.2, and the second biological condition may be a physiological pH of less than 6.0. More in particular, the first biological condition may be a physiological pH of more than 7.2, and the second biological condition may be a physiological pH of less than 5.7. For example, the first biological condition may be a physiological pH in the range of 7.2-7.4, and the second biological condition may be a physiological pH in the range of 6.0-6.5. For example, the first biological condition may be a physiological pH in the range of 7.2-7.4, and the second biological condition may be a physiological pH in the range of 5.0-6.0. For example, the first biological condition may be a physiological pH in the range of 7.2-7.4, and the second biological condition may be a physiological pH in the range of 5.3-5.7.

In another embodiment, the amino acid sequences directed against the serum protein may (further) be as generally described herein for the amino acid sequences of the invention. For example, they may be chosen from the group consisting of proteins and polypeptides with an immunoglobulin fold; molecules based on other protein scaffolds than immunoglobulins including but not limited to protein A domains, tendamistat, fibronectin, lipocalin, CTLA-4, T-cell receptors, designed ankyrin repeats and PDZ domains, and binding moieties based on DNA or RNA including but not limited to DNA or RNA aptamers; or from suitable parts, fragments, analogs, homologs, orthologs, variants or derivatives of such proteins or polypeptides; and in particular from the group consisting of antibodies and antibody fragments, binding units and binding molecules derived from antibodies or antibody fragments, and antibody fragments, binding units or binding molecules; or from suitable parts, fragments, analogs, homologs, orthologs, variants or derivatives of any of the foregoing.

Also, preferably, they are chosen from the group consisting of heavy chain variable domains, light chain variable domains, domain antibodies and proteins and peptides suitable for use as domain antibodies, single domain antibodies and proteins and peptides suitable for use as single domain antibodies, Nanobodies® and dAbs™; or from suitable parts, fragments, analogs, homologs, orthologs, variants or derivatives of any of the foregoing.

In particular, in this embodiment, the amino acid sequences of the invention (as well as compounds comprising the same, as defined herein) may be such that they bind to or otherwise associate with a serum protein (such as serum albumin) in such a way that, when the amino acid sequence is bound to or otherwise associated with said serum protein molecule (such as serum albumin) in a primate, it exhibits a serum half-life of at least about 50% (such as about 50% to 70%), preferably at least 60% (such as about 60% to 80%) or preferably at least 70% (such as about 70% to 90%), more preferably at least about 80% (such as about 80% to 90%) or preferably at least about 90% of the natural half-life of serum proteins such as serum albumin in said primate. For example, in this embodiment, the amino acid sequences of the invention may bind to or otherwise associate with human serum proteins such as serum albumin in such a way that, when the amino acid sequences are bound to or otherwise associated with a human serum protein such as serum albumin, the amino acid sequences exhibit a serum half-life in human of at least about 50% (such as about 50% to 70%), preferably at least 60% (such as about 60% to 80%) or preferably at least 70% (such as about 70% to 90%), more preferably at least about 80% (such as about 80% to 90%) or preferably at least about 90% of the natural half-life of said serum protein (such as human serum albumin). Also, preferably, in this embodiment, the amino acid sequences of the invention bind to said serum protein (such as human serum albumin) with a dissociation constant (K_{D}) and/or with a binding affinity (K_{A}) that is as defined herein. In man, the half-life of serum albumin is about 19 days (Peters T (1996) All About Albumin. Academic Press, San Diego).

This in vivo half-life in primates makes the amino acid sequences of the invention ideal candidates to prolong the serum half-life of therapeutics attached thereto. A long serum half-life of the combined amino acid sequence and therapeutics according to the invention in turn allows for reduced frequencies of administration and/or reduced amount to be administered, bringing about significant benefits for the subject to be treated.

This embodiment therefore also comprises compounds of the invention that comprise such an amino acid sequence and that have a half-life in human that is at least 80%, more preferably at least 90%, such as 95% or more or essentially the same as the half-life in human of the amino acid sequence present in said compound.

In one specific aspect of this embodiment, the amino acid sequences of the invention may be such that they are cross-reactive with the corresponding (i.e. orthologous) serum protein (such as serum albumin) from at least one further species of primate, and in particular with the corresponding (i.e. orthologous) serum protein from at least one species of primate that is chosen from the group consisting of monkeys from the genus Macaca (such as, and in particular, cynomologus monkeys (Macaca fascicularis) and/or rhesus monkeys (Macaca mulatta)) and baboon (Papio ursinus). Preferably, such cross-reactive amino acid sequences are further such that they exhibit a serum half-life in said primate of at least about 50% (such as about 50% to 70%), preferably at least 60% (such as about 60% to 80%) or preferably at least 70% (such as about 70% to 90%), more preferably at least about 80% (such as about 80% to 90%) or preferably at least about 90% of the natural half-life of the corresponding (i.e. orthologous) serum protein (such as serum albumin) in said primate. Such amino acid sequences of the invention also preferably bind to the corresponding (i.e. orthologous) serum protein (such as serum albumin) from said primate with a dissociation constant (K_{D}) and/or with a binding affinity (K_{A}) that is as defined herein.

This embodiment therefore also comprises compounds of the invention that comprise at least one amino acid sequence of the invention and that have a half-life in human and/or in said at least one species of primate that is at least 80%, more preferably at least 90%, such as 95% or more or essentially the same as the half-life in human and/or said species of primate, respectively, of the amino acid sequence of the invention present in said compound.

According to another preferred, but non-limiting aspect of this embodiment of the invention, the amino acid sequences of the invention are such that they bind to or otherwise associate with a human serum protein (such as human serum albumin) in such a way that, when the amino acid sequences are bound to or otherwise associated with said serum protein, the amino acid sequences exhibit a serum half-life in human of at least about 9 days (such as about 9 to 14 days), preferably at least about 10 days (such as about 10 to 15 days) or at least 11 days (such as about 11 to 16 days), more preferably at least about 12 days (such as about 12 to 18 days or more) or more than 14 days (such as about 14 to 19 days). Such amino acid sequences of the invention preferably can bind to said human serum protein (such as human serum albumin) with a dissociation constant (K_{D}) and/or with a binding affinity (K_{A}) that is as defined herein.

This embodiment therefore also comprises compounds of the invention that comprise such an amino acid sequence and that have a half-life in human that is at least 80%, more preferably at least 90%, such as 95% or more or essentially the same as the half-life in human of the amino acid sequence present in said compound.

In one specific but non-limiting aspect of this embodiment, the amino acid sequences of the invention may be such that they are cross-reactive with the corresponding (i.e. orthologous) serum protein (such as serum albumin) from at least one further species of primate, and in particular with the corresponding (i.e. orthologous) serum protein (such as serum albumin) from at least one species of primate that is chosen from the group consisting of monkeys from the genus Macaca (such as rhesus monkeys or cynomologus monkeys) and baboons. Preferably, such cross-reactive amino acid sequences exhibit a serum half-life in said primate of at least about 50% (such as about 50% to 70%), preferably at least 60% (such as about 60% to 80%) or preferably at least 70% (such as about 70% to 90%), more preferably at least about 80% (such as about 80% to 90%) or preferably at least about 90% of the natural half-life of the corresponding (i.e. orthologous) serum protein (such as serum albumin) in said primate. Such amino acid sequences of the invention also preferably bind to the corresponding (i.e. orthologous) serum protein (such as serum albumin) from said primate with a dissociation constant (K_{D}) and/or with a binding affinity (K_{A}) that is as defined herein.

This embodiment therefore also comprises compounds of the invention that comprise such an amino acid sequence and that have a half-life in human and/or in said at least one species of primate that is at least 80%, more preferably at least 90%, such as 95% or more or essentially the same as the half-life in human and/or said species of primate, respectively, of the amino acid sequence present in said compound.

In another specific, but non-limiting aspect of this embodiment, the amino acid sequences of the invention may be such that they bind to or otherwise associate with the corresponding (i.e. orthologous) serum protein (such as serum albumin) from at least one species of primate and that, when the half-life of the corresponding (i.e. orthologous) serum protein in the primate is at least about 10 days, such as between 10 and 15 days, for example about 11 to 13 days (by means of example, in rhesus monkeys, the expected half-life of serum albumin is between about 11 and 13 days, in particular about 11 to 12 days), have a serum half-life in said primate of least about 5 days (such as about 5 to 9 days), preferably at least about 6 days (such as about 6 to 10 days) or at least 7 days (such as about 7 to 11 days), more preferably at least about 8 days (such as about 8 to 12 days) or more than 9 days (such about 9 to 12 days or more). Such amino acid sequences of the invention are preferably further such that they bind to serum albumin from said species of primate with a dissociation constant (K_{D}) and/or with a binding affinity (K_{A}) that is as defined herein. In one specifically preferred aspect of this embodiment, such amino acid sequences are cross-reactive with human serum albumin, and more preferably bind to the corresponding (i.e. orthologous) serum protein (such as serum albumin) with a dissociation constant (K_{D}) and/or with a binding affinity (K_{A}) that is as defined herein.

This embodiment also comprises compounds of the invention that comprise such an amino acid sequence and that have a half-life in said at least one species of primate that is at least 80%, more preferably at least 90%, such as 95% or more or essentially the same as the half-life in said species of primate of the amino acid sequence present in said compound.

In another specific, but non-limiting aspect of this embodiment, the amino acid sequences of the invention may further be such that they bind to or otherwise associate the corresponding (i.e. orthologous) serum protein (such as serum albumin) from at least one species of primate and that, when the half-life of the corresponding (i.e. orthologous) serum protein (such as serum albumin) in the primate is at least about 13 days, such as between 13 and 18 days (by means of example, in baboons, the half-life of serum albumin is at least about 13 days, and usually about 16-18 days), have a serum half-life in said primate of least about 7 days (such as about 7 to 13 days), preferably at least about 8 days (such as about 8 to 15 days) or at least 9 days (such as about 9 to 16 days), more preferably at least about 10 days (such as about 10 to 16 days or more) or more than 13 days (such as about 13 to 18 days). Such amino acid sequences of the invention are preferably further such that they bind to the corresponding (i.e. orthologous) serum protein (such as serum albumin) from said species of primate with a dissociation constant (K_{D}) and/or with a binding affinity (K_{A}) that is as defined herein.

This embodiment also comprises compounds of the invention that comprise such an amino acid sequence and that have a half-life in said at least one species of primate that is at least 80%, more preferably at least 90%, such as 95% or more or essentially the same as the half-life in said species of primate of the amino acid sequence present in said compound.

In another specific, but non-limiting aspect of this embodiment, the amino acid sequences of the invention may be further such that they:
a) bind to or otherwise associate with a human serum protein (such as serum albumin) in such a way that, when the amino acid sequences are bound to or otherwise associated with said human serum protein, the amino acid sequences exhibit a serum half-life in human of at least about 9 days (such as about 9 to 14 days), preferably at least about 10 days (such as about 10 to 15 days) or at least 11 days (such as about 11 to 16 days), more preferably at least about 12 days (such as about 12 to 18 days or more) or more than 14 days (such as about 14 to 19 days); and
b) are cross-reactive with the corresponding (i.e. orthologous) serum protein (such as serum albumin) from at least one primate chosen from species of the genus Macaca (and in particular with the corresponding (i.e. orthologous) serum protein from cynomologus monkeys and/or from rhesus monkeys); and
c) have a serum half-life in said primate of at least about 5 days (such as about 5 to 9 days), preferably at least about 6 days (such as about 6 to 10 days) or at least 7 days (such as about 7 to 11 days), more preferably at least about 8 days (such as about 8 to 12 days) or more than 9 days (such about 9 to 12 days or more).

Preferably, such amino acid sequences bind to the human protein (such as human serum albumin) and/or to the corresponding (i.e. orthologous) serum protein (such as serum albumin) from said species of primate with a dissociation constant (K_{D}) and/or with a binding affinity (K_{A}) that is as defined herein.

This embodiment also comprises compounds of the invention that comprise such an amino acid sequence and that have a half-life in human and/or in said at least one species of primate that is at least 80%, more preferably at least 90%, such as 95% or more or essentially the same as the half-life in human and/or said species of primate, respectively, of the amino acid sequence present in said compound.

In another specific, but non-limiting aspect of this embodiment, the amino acid sequences of the invention may further be such that they:
a) bind to or otherwise associate with a human serum protein (such as serum albumin) in such a way that, when the amino acid sequences are bound to or otherwise associated with said human serum protein, the amino acid sequences exhibit a serum half-life in human of at least about 9 days (such as about 9 to 14 days), preferably at least about 10 days (such as about 10 to 15 days) or at least 11 days (such as about 11 to 16 days), more preferably at least about 12 days (such as about 12 to 18 days or more) or more than 14 days (such as about 14 to 19 days); and
b) are cross-reactive with the corresponding (i.e. orthologous) serum protein (such as serum albumin) from baboons; and
c) have a serum half-life in baboons of least about 7 days (such as about 7 to 13 days), preferably at least about 8 days (such as about 8 to 15 days) or at least 9 days (such as about 9 to 16 days), more preferably at least about 10 days (such as about 10 to 16 days or more) or more than 13 days (such as about 13 to 18 days).

Preferably, such amino acid sequences bind to the human serum protein (such as human serum albumin) and/or to the corresponding (i.e. orthologous) serum protein (such as serum albumin) from baboon with a dissociation constant (K_{D}) and/or with a binding affinity (K_{A}) that is as defined herein.

This embodiment also comprises compounds of the invention that comprise such an amino acid sequence and that have a half-life in human and/or in said at least one species of primate that is at least 80%, more preferably at least 90%, such as 95% or more or essentially the same as the half-life in human and/or said species of primate, respectively, of the amino acid sequence present in said compound.

Preferably, also, the half-life of the compounds, constructs, fusion proteins, etc. comprising at least one amino acid sequence of this embodiment is preferably at least 80%, more preferably at least 90%, such as 95% or more or essentially the same as the half-life of the amino acid sequence of the invention present therein (i.e. in the same primate).

In a particular, but non-limiting aspect of this embodiment of the invention, the amino acid sequences of the invention (or compounds comprising the same) are directed against a serum protein that binds or can bind to the FcRn receptor (e.g. as part of recycling of said serum protein) and are such that they can bind to or otherwise associate with said serum protein in such a way that, when the amino acid sequence or polypeptide construct is bound to or otherwise associated with a said serum protein molecule, the binding of said serum protein molecule to FcRn is not (significantly) reduced or inhibited. Some specific, but non-limiting serum proteins that can bind to FcRn include serum albumin and immunoglobulins, such as in particular IgG.

In a further aspect of this embodiment, the amino acid sequence of the invention (or compound comprising the same) can bind to or otherwise associate with a serum protein (such as serum albumin) in such a way that, when the amino acid sequence or polypeptide construct is bound to or otherwise associated with said serum protein molecule, the half-life of the serum protein molecule is not (significantly) reduced.

In a further aspect of this embodiment the amino acid sequence of the invention (or compound comprising the same) is capable of binding to amino acid residues on the serum protein that are not involved in binding of said serum protein to FcRn. For example, when the serum protein is serum albumin, the amino acid sequence of the invention (or compound comprising the same) is capable of binding to amino acid residues that do not form part of domain III of serum albumin.

In one aspect of this embodiment of the invention, the amino acid sequence is an immunoglobulin sequence or a fragment thereof, more specifically an immunoglobulin variable domain sequence or a fragment thereof, e.g. a VH-, VL- or VHH-sequence or a fragment thereof. The amino acid sequence of the invention may be a domain antibody, “dAb”, single domain antibody or Nanobody, or a fragment of any one thereof. The amino acid sequence of the invention may be a fully human, humanized, camelid, camelized human or humanized camelid sequence, and more specifically, may comprise 4 framework regions (FR1 to FR4 respectively) and 3 complementarity determining regions (CDR1 to CDR3 respectively).

More specifically, the amino acid sequence according to the invention may be a (single) domain antibody or a Nanobody.

Methods for generating the amino acid sequences directed against a serum protein for use in this embodiment may generally be as described herein, with the desired compound being the desired serum protein (such as serum albumin).

A further aspect of this embodiment relates to a compound of the invention that comprises at least one amino acid sequence according to this embodiment, which compound may optionally further comprise at least one therapeutic moiety, comprising therapeutic moieties selected from at least one of the group consisting of small molecules, polynucleotides, polypeptides or peptides. Such a compound of the invention is preferably such that it is suitable for administration to a primate with a frequency corresponding to not less than 50% (such as about 50% to 70%), preferably at least 60% (such as about 60% to 80%) or preferably at least 70% (such as about 70% to 90%), more preferably at least about 80% (such as about 80% to 90%) or preferably at least about 90% of the natural half-life of the serum protein (such as serum albumin) in said primate, or, alternatively, at intervals of at least 4 days (such as about 4 to 12 days or more), preferably at least 7 days (such as about 7 to 15 days or more), more preferably at least 9 days (such as about 9 to 17 days or more), such as at least 15 days (such as about 15 to 19 days or more, in particular for administration to man) or at least 17 days (such as about 17 to 19 days or more, in particular for administration to man); where such administrations are in particular made to maintain the desired level of the compound in the serum of the subject that is treated with the compound (such inter alia dependent on the compound used and/or the disease to be treated, as will be clear to the skilled person. The clinician or physician will be able to select the desired serum level and to select the dose(s) and/or amount(s) to be administered to the subject to be treated in order to achieve and/or to maintain the desired serum level in said subject, when the compound of the invention is administered at the frequencies mentioned herein. For example, such a dose can range between 1 times and 10 times the desired serum level, such as between 2 times and 4 times the desired serum level (in which the desired serum level is recalculated in a manner known per se so as to provide a corresponding dose to be administered).

Such compounds of the invention may also be formulated as unit doses that are intended and/or packaged (e.g. with suitable instructions for use) for administration at the aforementioned frequencies, and such unit doses and packaged products form further aspects of the invention. Another aspect of the invention relates to the use of a compound of the invention in providing such a unit dose or packaged product (i.e. by suitably formulating and/or packaging said compound).

In a particular aspect of this embodiment, the compound of the invention is a fusion protein or construct. In said fusion protein or construct the amino acid sequence of the invention may be either directly linked to the at least one therapeutic moiety or is linked to the at least one therapeutic moiety via a linker or spacer. A particular embodiment relates to a therapeutic moiety comprising an immunoglobulin sequence or a fragment thereof, more specifically a (single) domain antibody or a Nanobody.

In a specific aspect, this embodiment also relates to multivalent and multispecific Nanobody constructs, comprising at least one amino acid sequence of the invention which is a Nanobody and at least one further Nanobody. The Nanobody is either directly linked to the at least one further Nanobody or is linked to the at least one further Nanobody via a linker or spacer, preferably linked to the at least one further Nanobody via an amino acid sequence linker or spacer.

Also, as indicated herein, but without limitation, bispecific (or multispecific) compounds that conditionally binds to at least one serum protein and at least one (other) intended or desired molecule may find particular use in this embodiment of the invention. As such, the compound of this embodiment may be such that it binds to both the serum protein and the intended or desired molecule under the first biological condition (or alternatively, under the second biological condition), or may be such that it hinds to the serum protein under the first biological condition but binds to the other intended or desired molecule under the second biological condition. Thus, when changing from the first biological condition to the second biological condition, such a compound of this embodiment will therefore be released from the first serum protein molecule and bind to the intended or desired molecule (or vise versa).

Also, in such a bispecific molecule, the conditional binder that binds to the intended or desired molecule may itself form or function as a therapeutic moiety (in which case it may be as further described herein), and/or such a compound of the invention may contain one or more further therapeutic moieties (as defined herein).

Non-limiting examples of such bispecific compounds of this embodiment are also illustrated in FIG. 1, which is a non-limiting schematic drawing showing an example of the possible interaction(s) between FcRn, a serum protein binding to FcRn (such as serum albumin or IgG), a bispecific compound of the invention (in particular, a bispecific compound according to the specific embodiment for extending half-life as described herein) and an antigen (i.e. as a second intended or desired molecule). Also, Tables 1-3 outline different non-limiting examples of the way in which a bispecific compound of the invention (in particular, a bispecific compound according to the specific embodiment for extending half-life as described herein) can bind to a serum protein (i.e. as a first intended or desired molecule) and to an antigen (as a second intended or desired molecule). Further reference is made to the detailed description herein.

Furthermore, this embodiment relates to nucleotide sequence or nucleic acid that encode an amino acid sequence according to this embodiment, or the amino acid sequence of a compound according to this embodiment, or the multivalent and multispecific Nanobody of this embodiment. This embodiment also provides hosts or host cells that contain a nucleotide sequence or nucleic acid of this embodiment and/or that express (or are capable of expressing) an amino acid sequence of this embodiment, or the amino acid sequence of a compound according to this embodiment, or the multivalent and multispecific Nanobody of this embodiment.

Moreover, this embodiment relates to method for preparing an amino acid sequence, compound, or multivalent and multispecific Nanobody of this embodiment comprising cultivating or maintaining a host cell of this embodiment under conditions such that said host cell produces or expresses the said product, and optionally further comprises the said product so produced.

In one embodiment, this embodiment relates to a pharmaceutical composition comprising one or more selected from the group consisting of the amino acid sequence, compound, or multivalent and multispecific Nanobody of this embodiment, wherein said pharmaceutical composition is suitable for administration to a primate at intervals of at least about 50% of the natural half-life of the serum protein in said primate. The pharmaceutical composition may further comprise at least one pharmaceutically acceptable carrier, diluent or excipient.

This embodiment also encompasses medical uses and methods of treatment encompassing the amino acid sequence, compound or multivalent and multispecific Nanobody of this embodiment, wherein said medical use or method is characterized in that said medicament is suitable for administration at intervals of at least about 50% of the natural half-life of the serum protein in said primate, and the method comprises administration at a frequency of at least about 50% of the natural half-life of the serum protein in said primate.

This embodiment also relates to methods for extending or increasing the serum half-life of a therapeutic. The methods include contacting the therapeutic with any of the foregoing amino acid sequences, compounds, fusion proteins or constructs of this embodiment (including multivalent and multispecific Nanobodies), such that the therapeutic is bound to or otherwise associated with the amino acid sequences, compounds, fusion proteins or constructs of this embodiment. In some embodiments, the therapeutic is a biological therapeutic, preferably a peptide or polypeptide, in which case the step of contacting the therapeutic can include preparing a fusion protein by linking the peptide or polypeptide with the amino acid sequence, compound, fusion proteins or constructs of this embodiment.

These methods can further include administering the therapeutic to a primate after the therapeutic is bound to or otherwise associated with the amino acid sequence, compound, fusion protein or construct of this embodiment. In such methods, the serum half-life of the therapeutic in the primate is at least 1.5 times the half-life of therapeutic per se, or is increased by at least 1 hour compared to the half-life of therapeutic per se. In some preferred embodiments, the serum half-life of the therapeutic in the primate is at least 2 times, at least 5 times, at least 10 times or more than 20 times greater than the half-life of the corresponding therapeutic moiety per se. In other preferred embodiments, the serum half-life of the therapeutic in the primate is increased by more than 2 hours, more than 6 hours or more than 12 hours compared to the half-life of the corresponding therapeutic moiety per se.

Preferably, the serum half-life of the therapeutic in the primate is increased so that the therapeutic has a half-life that is as defined herein for the compounds of this embodiment (i.e. in human and/or in at least one species of primate).

In another aspect, this embodiment relates to a method for modifying a therapeutic such that the desired therapeutic level of said therapeutic is, upon suitable administration of said therapeutic so as to achieve said desired therapeutic level, maintained for a prolonged period of time.

The methods include contacting the therapeutic with any of the foregoing amino acid sequences, compounds, fusion proteins or constructs of this embodiment (including multivalent and multispecific Nanobodies), such that the therapeutic is bound to or otherwise associated with the amino acid sequences, compounds, fusion proteins or constructs of this embodiment. In some embodiments, the therapeutic is a biological therapeutic, preferably a peptide or polypeptide, in which case the step of contacting the therapeutic can include preparing a fusion protein by linking the peptide or polypeptide with the amino acid sequence, compound, fusion proteins or constructs of this embodiment.

These methods can further include administering the therapeutic to a primate after the therapeutic is bound to or otherwise associated with the amino acid sequence, compound, fusion protein or construct of this embodiment, such that the desired therapeutic level is achieved upon such administration. In such methods, the time that the desired therapeutic level of said therapeutic is maintained upon such administration is at least 1.5 times the half-life of therapeutic per se, or is increased by at least 1 hour compared to the half-life of therapeutic per se. In some preferred embodiments, the time that the desired therapeutic level of said therapeutic is maintained upon such administration is at least 2 times, at least 5 times, at least 10 times or more than 20 times greater than, the half-life of the corresponding therapeutic moiety per se. In other preferred embodiments, the time that the desired therapeutic level of said therapeutic is maintained upon such administration is increased by more than 2 hours, more than 6 hours or more than 12 hours compared to the half-life of the corresponding therapeutic moiety per se.

Preferably, the time that the desired therapeutic level of said therapeutic is maintained upon such administration is increased such that the therapeutic can be administered at a frequency that is as defined herein for the compounds of this embodiment.

In another aspect, this embodiment relates to the use of a compound of this embodiment (as defined herein) for the production of a medicament that increases and/or extends the level of the therapeutic agent in said compound or construct in the serum of a patient such that said therapeutic agent in said compound or construct is capable of being administered at a lower dose as compared to the therapeutic agent alone (i.e. at essentially the same frequency of administration).

The amino acid sequences of this embodiment are also preferably such that they can bind to or otherwise associate with the serum protein (such as serum albumin) in such a way that, when the amino acid sequence or polypeptide construct is bound to or otherwise associated with the serum protein molecule in a primate, they exhibit a serum half-life of at least about 50% of the natural half-life of the serum protein in said primate, preferably at least about 60%, preferably at least about 70%, more preferably at least about 80% and most preferably at least about 90%.

The serum half-life of the amino acid sequences of this embodiment after administration to a primate may be at least 50%, 55%, 60%, 65%, 70%, 75%, 80%, 85%, 90%, 95% or at least 100% of the natural half-life of the serum protein in said primate.

By “natural serum half-life of the serum protein in said primate” is meant the serum half-life as defined below, which the serum protein has in healthy individuals under physiological conditions. Taking serum albumin as an example of the serum protein, the natural serum half-life of serum albumin in humans is 19 days. Smaller primates are known to have shorter natural half-lives of serum albumin, e.g. in the range of 8 to 19 days. Specific half-lives of serum albumin may be at least 4, 5, 6, 7, 8, 9, 10, 11, 12, 13, 14, 15, 16, 17, 18, or 19 days or more.

From this it follows, that for example in a human individual, an amino acid sequence of this embodiment shows a serum half-life in association with serum albumin of at least about 50% of 19 days, i.e. 7.6 days. In smaller primates, the serum half-life may be shorter in days, depending on the natural half-lives of serum albumin in these species.

In the present description, the term “primate” refers to both species of monkeys an apes, and includes species of monkeys such as monkeys from the genus Macaca (such as, and in particular, cynomologus monkeys (Macaca fascicularis) and/or rhesus monkeys (Macaca mulatta)) and baboon (Papio ursinus)), as well as marmosets (species from the genus Callithrix), squirrel monkeys (species from the genus Saimiri) and tamarins (species from the genus Saguinus), as well as species of apes such as chimpanzees (Pan troglodytes), and also includes man. Humans are the preferred primate according to this embodiment.

The half-life of an amino acid sequence or compound can generally be defined as the time taken for the serum concentration of the polypeptide to be reduced by 50%, in vivo, for example due to degradation of the sequence or compound and/or clearance or sequestration of the sequence or compound by natural mechanisms. The half-life of the amino acid sequences of this embodiment (and of compounds comprising the same) in the relevant species of primate can be determined in any manner known per se, such as by pharmacokinetic analysis. Suitable techniques will be clear to the person skilled in the art, and may for example generally involve the steps of suitably administering to the primate a suitable dose of the amino acid sequence or compound to be treated; collecting blood samples or other samples from said primate at regular intervals; determining the level or concentration of the amino acid sequence or compound of this embodiment in said blood sample; and calculating, from (a plot of) the data thus obtained, the time until the level or concentration of the amino acid sequence or compound of this embodiment has been reduced by 50% compared to the initial level upon dosing. Reference is for example made to standard handbooks, such as Kenneth, A et al: Chemical Stability of Pharmaceuticals: A Handbook for Pharmacists and in Peters et al, Pharmacokinete analysis: A Practical Approach (1996). Reference is also made to “Pharmacokinetics”, M Gibaldi & D Perron, published by Marcel Dekker, 2nd Rev. edition (1982).

As described on pages 6 and 7 of WO 04/003019 and in the further references cited therein, the half-life can be expressed using parameters such as the t1/2-alpha, t1/2-beta and the area under the curve (AUC). In the present specification, an “increase in half-life” refers to an increase in any one of these parameters, such as any two of these parameters, or essentially all three these parameters. An “increase in half-life” in particular refers to an increase in the t1/2-beta, either with or without an increase in the t1/2-alpha and/or the AUC or both.

In another aspect, the amino acid sequences of this embodiment, and in particular immunoglobulin sequences of this embodiment, and more in particular immunoglobulin variable domain sequences of this embodiment, directed against a serum protein (such as serum albumin, preferably human serum albumin), are such that they that have a half-life in rhesus monkeys of at least about 4, preferably at least about 7, more preferably at least about 9 days.

In yet another aspect, the amino acid sequences of this embodiment are such that they have a half-life in human of at least about 7, preferably at least about 15, more preferably at least about 17 days. This embodiment also relates to compounds of this embodiment that have a half-life in human that is at least 80%, more preferably at least 90%, such as 95% or more or essentially the same as the half-life of the amino acid sequence of this embodiment present in said compound. More in particular, this embodiment also relates to compounds of this embodiment that have a half-life in human of at least about 7, preferably at least about 15, more preferably at least about 17 days.

This embodiment also provides compounds comprising the amino acid sequence of this embodiment, in particular compounds comprising at least one therapeutic moiety in addition to the amino acid sequence of this embodiment. The compounds according to this embodiment are characterized by exhibiting a comparable serum half-life in primates to the amino acid sequence of this embodiment, more preferable a half-life which is at least the serum half-life of the amino acid sequence of this embodiment, and more preferably a half-life which is higher than the half-life of the amino acid sequence of this embodiment in primates.

In one aspect, this embodiment achieves this objective by providing the amino acid sequences disclosed herein, that can bind to a serum protein that can bind to FcRn, which amino acid sequences are further such that they can bind to or otherwise associate with the serum protein (such as serum albumin) in such a way that, when the amino acid sequence or polypeptide construct is bound to or otherwise associated with the serum protein molecule, the binding of said serum protein molecule to FcRn is not (significantly) reduced or inhibited (i.e. compared to the binding of said serum protein molecule to FcRn when the amino acid sequence or polypeptide construct is not bound thereto). In this aspect of this embodiment, by “not significantly reduced or inhibited” is meant that the binding affinity for serum protein to FcRn (as measured using a suitable assay, such as SPR) is not reduced by more than 50%, preferably not reduced by more than 30%, even more preferably not reduced by more than 10%, such as not reduced by more than 5%, or essentially not reduced at all. In this aspect of this embodiment, “not significantly reduced or inhibited” may also mean (or additionally mean) that the half-life of the serum protein molecule is not significantly reduced (as defined below).

When in this description, reference is made to binding, such binding is preferably specific binding, as normally understood by the skilled person.

When an amino acid sequence as described herein is a monovalent immunoglobulin sequence (for example, a monovalent Nanobody), said monovalent immunoglobulin sequence preferably binds to human serum albumin under the first biological condition with a dissociation constant (K_{D}) of 10⁻⁵ to 10⁻¹² moles/liter or less, and preferably 10⁻⁷ to 10⁻¹² moles/liter or less and more preferably 10⁻⁸ to 10⁻¹² moles/liter (i.e. with an association constant (K_{A}) of 10⁵ to 10¹² liter/moles or more, and preferably 10⁷ to 10¹² liter/moles or more and more preferably 10⁸ to 10¹² liter/moles, and/or with a binding affinity (K_{A}) of at least 10⁷ M⁻¹, preferably at least 10⁸ M⁻¹, more preferably at least 10⁹ M⁻¹, such as at least 10¹² M⁻¹. Any Kₙ value greater than 10⁴ mol/liter (or any K_{A} value lower than 10⁴ M⁻¹) liters/mol is generally considered to indicate non-specific binding. Preferably, a monovalent immunoglobulin sequence of this embodiment will bind to the desired serum protein under the first biological condition with an affinity less than 3000 nM, preferably less than 300 nM, more preferably less than 30 nM, such as less than 3 nM. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.

In another aspect, the amino acid sequences (and in particular immunoglobulin sequences, and more in particular immunoglobulin variable domain sequences) of this embodiment, are further such that they can bind to or otherwise associate with a serum protein (such as serum albumin) in such a way that, when the amino acid sequence or polypeptide construct is bound to or otherwise associated with said serum protein molecule, the half-life of said serum protein molecule is not (significantly) reduced (i.e. compared to the half-life of the serum protein molecule when the amino acid sequence or polypeptide construct is not bound thereto). In this aspect of this embodiment, by “not significantly reduced” is meant that the half-life of the serum protein molecule (as measured using a suitable technique known per se) is not reduced by more than 50%, preferably not reduced by more than 30%, even more preferably not reduced by more than 10%, such as not reduced by more than 5%, or essentially not reduced at all.

In another aspect, the amino acid sequences (and in particular immunoglobulin sequences, and more in particular immunoglobulin variable domain sequences) of this embodiment may be directed against serum proteins that can bind to FcRn, and may be further such that they are capable of binding to amino acid residues on the serum protein molecule (such as amino acid residues on serum albumin) that are not involved in binding of said serum protein to FcRn. In particular, according to this aspect of this embodiment, when the amino acid sequences of this embodiment are directed against serum albumin, they are such that they are capable of binding to amino acid sequences of serum albumin that do not form part of domain III of serum albumin. For example, but without being limited thereto, this aspect of this embodiment provides amino acid sequences that are capable of binding to amino acid sequences of serum albumin that form part of domain I and/or domain II.

The amino acid sequences of this embodiment are preferably (single) domain antibodies or suitable for use as (single) domain antibodies, and as such may be heavy chain variable domain sequence (VH sequence) or a light chain variable domain sequence (VL sequence), and preferably are VH sequences. The amino acid sequences may for example be so-called “dAbs”.

However, according to a particularly preferred embodiment, the amino acid sequences of the present invention are Nanobodies. For a further description and definition of Nanobodies, as well as of some of the further terms used in the present description (such as, for example and without limitation, the term “directed against”) reference is made to the copending patent applications by Ablynx N.V. (such as WO 06/040153 and the copending International application PCT/EP2006/004678)); as well as the further prior art cited therein.

As such, they may be Nanobodies belonging to the “KERE”-class, to the “GLEW”-class or to the “103-P,R,S”-class (again as defined in the copending patent applications by Ablynx N.V.).

Preferably, the amino acid sequences of the present invention are humanized Nanobodies (again as defined in the copending patent applications by Ablynx N.V.).

The amino acid sequences disclosed herein can be used with advantage as a fusion partner in order to increase the half-life of therapeutic moieties such as proteins, compounds (including, without limitation, small molecules) or other therapeutic entities.

Thus, in another aspect, this embodiment provides proteins or polypeptides that comprise or essentially consist of an amino acid sequence as disclosed herein. In particular, this embodiment provides protein or polypeptide constructs that comprise or essentially consist of at least one amino acid sequence of this embodiment that is linked to at least one therapeutic moiety, optionally via one or more suitable linkers or spacers. Such protein or polypeptide constructs may for example (without limitation) be a fusion protein, as further described herein.

This embodiment further relates to therapeutic uses of protein or polypeptide constructs or fusion proteins and constructs and to pharmaceutical compositions comprising such protein or polypeptide constructs or fusion proteins.

In some embodiments the at least one therapeutic moiety comprises or essentially consists of a therapeutic protein, polypeptide, compound, factor or other entity. In a preferred embodiment the therapeutic moiety is directed against a desired antigen or target, is capable of binding to a desired antigen (and in particular capable of specifically binding to a desired antigen), and/or is capable of interacting with a desired target. In another embodiment, the at least one therapeutic moiety comprises or essentially consists of a therapeutic protein or polypeptide. In a further embodiment, the at least one therapeutic moiety comprises or essentially consists of an immunoglobulin or immunoglobulin sequence (including but not limited to a fragment of an immunoglobulin), such as an antibody or an antibody fragment (including but not limited to an ScFv fragment). In yet another embodiment, the at least one therapeutic moiety comprises or essentially consists of an antibody variable domain, such as a heavy chain variable domain or a light chain variable domain.

In a preferred embodiment, the at least one therapeutic moiety comprises or essentially consists of at least one domain antibody or single domain antibody, “dAb” or Nanobody®. According to this embodiment, the amino acid sequence of this embodiment is preferably also a domain antibody or single domain antibody, “dAb” or Nanobody, so that the resulting construct or fusion protein is a multivalent construct (as described herein) and preferably a multispecific construct (also as defined herein) comprising at least two domain antibodies, single domain antibodies, “dAbs” or Nanobodies® (or a combination thereof), at least one of which is an amino acid sequence of this embodiment.

In a specific embodiment, the at least one therapeutic moiety comprises or essentially consists of at least one monovalent Nanobody® or a bivalent, multivalent, bispecific or multi specific Nanobody® construct. According to this embodiment, the amino acid sequence of this embodiment is preferably also a Nanobody, so that the resulting construct or fusion protein is a multivalent Nanobody construct (as described herein) and preferably a multispecific Nanobody construct (also as defined herein) comprising at least two Nanobodies, at least one of which is an amino acid sequence of this embodiment.

According to one embodiment of this embodiment, the amino acid sequence of this embodiment is a humanized Nanobody.

Also, when the amino acid sequences, proteins, polypeptides or constructs of this embodiment are intended for pharmaceutical or diagnostic use, the aforementioned are preferably directed against a human serum protein, such as human serum albumin.

When the amino acid sequence is an immunoglobulin sequence such as a immunoglobulin variable domain sequence, a suitable (i.e. suitable for the purposes mentioned herein) fragment of such a sequence may also be used. For example, when the amino acid sequence is a Nanobody, such a fragment may essentially be as described in WO 04/041865.

This embodiment also relates to a protein or polypeptide that comprises or essentially consists of an amino acid sequence as described herein, or a suitable fragment thereof.

The amino acid sequences of this embodiment may also contain one or more additions binding sites for one or more other antigens, antigenic determinants, proteins, polypeptides, or other compounds.

As mentioned herein, the amino acid sequences described herein can be used with advantage as a fusion partner in order to increase the half-life of therapeutic moieties such as proteins, compounds (including, without limitation, small molecules) or other therapeutic entities. Thus, one embodiment of this embodiment relates to a construct or fusion protein that comprises at least one amino acid sequence of this embodiment and at least one therapeutic moieties. Such a construct or fusion protein preferably has increased half-life, compared to the therapeutic moiety per se. Generally, such fusion proteins and constructs can be (prepared and used) as described in the prior art cited above, but with an amino acid sequence of this embodiment instead of the half-life increasing moieties described in the prior art.

Generally, the constructs or fusion proteins described herein preferably have a half-life that is at least 1.5 times, preferably at least 2 times, such as at least 5 times, for example at least 10 times or more than 20 times, greater than the half-life of the corresponding therapeutic moiety per se.

Also, preferably, any such fusion protein or construct has a half-life that is increased with more than 1 hour, preferably more than 2 hours, more preferably of more than 6 hours, such as of more than 12 hours, compared to the half-life of the corresponding therapeutic moiety per se.

Also, preferably, any fusion protein or construct has a half-life that is more than 1 hour, preferably more than 2 hours, more preferably of more than 6 hours, such as of more than 12 hours, and for example of about one day, two days, one week, two weeks or three weeks, and preferably no more than 2 months, although the latter may be less critical.

Also, as mentioned above, when the amino acid sequence of this embodiment is a Nanobody, it can be used to increase the half-life of other immunoglobulin sequences, such as domain antibodies, single domain antibodies, “dAbs” or Nanobodies.

Thus, one embodiment of this embodiment relates to a construct or fusion protein that comprises at least one amino acid sequence of this embodiment and at least one immunoglobulin sequence, such as a domain antibodies, single domain antibodies, “dAbs” or Nanobodies. The immunoglobulin sequence is preferably directed against a desired target (which is preferably a therapeutic target), and/or another immunoglobulin sequence that useful or suitable for therapeutic, prophylactic and/or diagnostic purposes.

Thus, in another aspect, this embodiment relates to a multi specific (and in particular bispecific) Nanobody constructs that comprises at least one Nanobody as described herein, and at least one other Nanobody, in which said at least one other Nanobody is preferably directed against a desired target (which is preferably a therapeutic target), and/or another Nanobody that useful or suitable for therapeutic, prophylactic and/or diagnostic purposes.

For a general description of Nanobodies and of multivalent and multispecific polypeptides containing one or more Nanobodies and their preparation, reference is made to the co-pending applications by Ablynx N.V. such as WO 06/040153 and the copending International application PCT/EP2006/004678 (as well as the further prior art cited in these applications), and also to for example Conrath et al., J. Biol. Chem., Vol. 276, 10. 7346-7350, 2001; Muyldermans, Reviews in Molecular Biotechnology 74 (2001), 277-302; as well as to for example WO 96/34103 and WO 99/23221. Some other examples of some specific multispecific and/or multivalent polypeptide of this embodiment can be found in the co-pending applications by Ablynx N.V. In particular, for a general description of multivalent and multispecific constructs comprising at least one Nanobody against a serum protein for increasing the half-life, of nucleic acids encoding the same, of compositions comprising the same, of the preparation of the aforementioned, and of uses of the aforementioned, reference is made to the International application WO 04/041865 by Ablynx N.V. The amino acid sequences described herein can generally be used analogously to the half-life increasing Nanobodies described therein.

In one non-limiting embodiment, said other Nanobody is directed against tumor necrosis factor alpha (TNF-alpha), in monomeric and/or multimeric (i.e. trimeric) form. Some examples of such Nanobody constructs can be found in the copending International application by Ablynx N.V. entitled “Improved Nanobodies™ against. Tumor Necrosis Factor-alpha”, which has the same priority and the same international filing date as the present application.

This embodiment also relates to nucleotide sequences or nucleic acids that encode amino acid sequences, compounds, fusion proteins and constructs described herein. This embodiment further includes genetic constructs that include the foregoing nucleotide sequences or nucleic acids and one or more elements for genetic constructs known per se. The genetic construct may be in the form of a plasmid or vector. Again, such constructs can be generally as described in the co-pending patent applications by Ablynx N.V. and prior art mentioned herein, and in the further prior art cited therein.

This embodiment also relates to hosts or host cells that contain such nucleotide sequences or nucleic acids, and/or that express (or are capable of expressing), the amino acid sequences, compounds, fusion proteins and constructs described herein. Again, such host cells can be generally as described in the co-pending patent applications by Ablynx N.V. and prior an mentioned herein, and in the further prior art cited therein.

This embodiment also relates to a method for preparing an amino acid sequence, compound, fusion protein or construct as described herein, which method comprises cultivating or maintaining a host cell as described herein under conditions such that said host cell produces or expresses an amino acid sequence, compound, fusion protein or construct as described herein, and optionally further comprises isolating the amino acid sequence, compound, fusion protein or construct so produced. Again, such methods can be performed as generally described in the co-pending patent applications by Ablynx N.V. and prior art mentioned herein, and in the further prior art cited therein.

This embodiment also relates to a pharmaceutical composition that comprises at least one amino acid sequence, compound, fusion protein or construct as described herein, and optionally at least one pharmaceutically acceptable carrier, diluent or excipient. Such preparations, carriers, excipients and diluents may generally be as described in the co-pending patent applications by Ablynx N.V. and prior art mentioned herein, and in the further prior art cited therein.

However, since the amino acid sequences, compounds, fusion proteins or constructs described herein have an increased half-life, they are preferably administered to the circulation. As such, they can be administered in any suitable manner that allows the amino acid sequences, compound, fusion proteins or constructs to enter the circulation, such as intravenously, via injection or infusion, or in any other suitable manner (including oral administration, administration through the skin, transmucosal administration, intranasal administration, administration via the lungs, etc) that allows the amino acid sequences, compounds, fusion proteins or constructs to enter the circulation. Suitable methods and routes of administration will be clear to the skilled person, again for example also from the teaching of WO 04/041862.

Thus, in another aspect, this embodiment relates to a method for the prevention and/or treatment of at least one disease or disorder that can be prevented or treated by the use of a compound, fusion protein or construct as described herein, which method comprises administering, to a subject in need thereof, a pharmaceutically active amount of an amino acid sequence, compound, fusion protein or construct of this embodiment, and/or of a pharmaceutical composition comprising the same. The diseases and disorders that can be prevented or treated by the use of an amino acid sequence, compound, fusion protein or construct as described herein will generally be the same as the diseases and disorders that can be prevented or treated by the use of the therapeutic moiety that is present in the amino acid sequence, compound, fusion protein or construct of this embodiment.

The subject to be treated may be any primate, but is in particular a human being. As will be clear to the skilled person, the subject to be treated will in particular be a person suffering from, or at risk from, the diseases and disorders mentioned herein.

More specifically, the present invention relates to a method of treatment wherein the frequency of administering the amino acid sequence, compound, fusion protein or construct of this embodiment is at least 50% of the natural half-life of the serum protein against which the amino acid sequence, compound, fusion protein or construct of this embodiment is directed, preferably at least 60%, preferably at least 70%, more preferably at least 80% and most preferably at least 90%.

Specific frequencies of administration to a primate, which are within the scope of the present invention are at least 50%, 55%, 60%, 65%, 70%, 75%, 80%, 85%, 90%, 95% or at least 100% of the natural half-life of the serum protein against which the amino acid sequence, compound, fusion protein or construct of this embodiment is directed.

in other words, specific frequencies of administration which are within the scope of the present invention are every 4, 5, 6, 7, 8, 9, 10, 11, 12, 13, 14, 15, 16, 17, 18, or 19 days.

Without limitation, the frequencies of administration referred to above are in particular suited for maintaining a desired level of the amino acid sequence, compound, fusion protein or construct in the serum of the subject treated with the amino acid sequence, compound, fusion protein or construct, optionally after administration of one or more (initial) doses that are intended to establish said desired serum level. As will be clear to the skilled person, the desired serum level may inter alia be dependent on the amino acid sequence, compound, fusion protein or construct used and/or the disease to be treated. The clinician or physician will be able to select the desired serum level and to select the dose(s) and/or amount(s) to be administered to the subject to be treated in order to achieve and/or to maintain the desired serum level in said subject, when the amino acid sequence, compound, fusion protein or construct of this embodiment is administered at the frequencies mentioned herein.

In the context of the present invention, the term “prevention and/or treatment” not only comprises preventing and/or treating the disease, but also generally comprises preventing the onset of the disease, slowing or reversing the progress of disease, preventing or slowing the onset of one or more symptoms associated with the disease, reducing and/or alleviating one or more symptoms associated with the disease, reducing the severity and/or the duration of the disease and/or of any symptoms associated therewith and/or preventing a further increase in the severity of the disease and/or of any symptoms associated therewith, preventing, reducing or reversing any physiological damage caused by the disease, and generally any pharmacological action that is beneficial to the patient being treated.

The subject to be treated may be any primate, but is in particular a human being. As will be clear to the skilled person, the subject to be treated will in particular be a person suffering from, or at risk from, the diseases and disorders treatable by the therapeutic moiety mentioned herein.

In another embodiment, this embodiment relates to a method for immunotherapy, and in particular for passive immunotherapy, which method comprises administering, to a subject suffering from or at risk of the diseases and disorders mentioned herein, a pharmaceutically active amount of an amino acid sequence, compound, fusion protein or construct of this embodiment, and/or of a pharmaceutical composition comprising the same.

This embodiment also relates to methods for extending or increasing the serum half-life of a therapeutic. In these methods, the therapeutic is contacted with any of the amino acid sequences, compounds, fusion proteins or constructs of this embodiment, including multivalent and multispecific Nanobodies, such that the therapeutic is bound to or otherwise associated with the amino acid sequences, compounds, fusion proteins or constructs.

The therapeutic and the amino acid sequences, compounds, fusion proteins or constructs can be bound or otherwise associated in various ways known to the skilled person. In the case of biological therapeutics, such as a peptide or polypeptide, the therapeutic can be fused to the amino acid sequences, compounds, fusion proteins or constructs according to methods known in the art. The therapeutic can be directly fused, or fused using a spacer or linker molecule or sequence. The spacer or linker are, in preferred embodiments, made of amino acids, but other non-amino acid spacers or linkers can be used as is well known in the art. Thus, the step of contacting the therapeutic can include preparing a fusion protein by linking the peptide or polypeptide with the amino acid sequences, compounds, fusion proteins or constructs of this embodiment, including multivalent and multispecific Nanobodies.

The therapeutic also can be bound directly by the amino acid sequences, compounds, fusion proteins or constructs of this embodiment. As one example, a multivalent and multispecific Nanobody can include at least one variable domain that binds the serum protein (such as serum albumin) and at least one variable domain that binds the therapeutic.

The methods for extending or increasing serum half-life of a therapeutic can further include administering the therapeutic to a primate after the therapeutic is bound to or otherwise associated with the amino acid sequence, compound, fusion proteins or constructs of this embodiment. In such methods the half-life of the therapeutic is extended or increased by significant amounts, as is described elsewhere herein.

The amino acid sequence, compound, fusion protein or construct and/or the compositions comprising the same are administered according to a regime of treatment that is suitable for preventing and/or treating the disease or disorder to be prevented or treated. The clinician will generally be able to determine a suitable treatment regimen, depending on factors such as the disease or disorder to be prevented or treated, the severity of the disease to be treated and/or the severity of the symptoms thereof, the specific Nanobody or polypeptide of this embodiment to be used, the specific route of administration and pharmaceutical formulation or composition to be used, the age, gender, weight, diet, general condition of the patient, and similar factors well known to the clinician.

Generally, the treatment regimen will comprise the administration of one or more amino acid sequences, compounds, fusion proteins or constructs of this embodiment, or of one or more compositions comprising the same, in one or more pharmaceutically effective amounts or doses. The specific amount(s) or doses to administered can be determined by the clinician, again based on the factors cited above.

Generally, for the prevention and/or treatment of the diseases and disorders mentioned herein and depending on the specific disease or disorder to be treated, the potency and/or the half-life of the specific amino acid sequences, compounds, fusion proteins or constructs to be used, the specific route of administration and the specific pharmaceutical formulation or composition used, the Nanobodies and polypeptides of this embodiment will generally be administered in an amount between 1 gram and 0.01 microgram per kg body weight per day, preferably between 0.1 gram and 0.1 microgram per kg body weight per day, such as about 1, 10, 100 or 1000 microgram per kg body weight per day, either continuously (e.g. by infusion), as a single daily dose or as multiple divided doses during the day. The clinician will generally be able to determine a suitable daily dose, depending on the factors mentioned herein. It will also be clear that in specific cases, the clinician may choose to deviate from these amounts, for example on the basis of the factors cited above and his expert judgment. Generally, some guidance on the amounts to be administered can be obtained from the amounts usually administered for comparable conventional antibodies or antibody fragments against the same target administered via essentially the same route, taking into account however differences in affinity/avidity, efficacy, biodistribution, half-life and similar factors well known to the skilled person.

Usually, in the above method, a single Nanobody or polypeptide of this embodiment will be used. It is however within the scope of this embodiment to use two or more Nanobodies and/or polypeptides of this embodiment in combination.

The Nanobodies and polypeptides of this embodiment may also be used in combination with one or more further pharmaceutically active compounds or principles, i.e. as a combined treatment regimen, which may or may not lead to a synergistic effect. Again, the clinician will be able to select such further compounds or principles, as well as a suitable combined treatment regimen, based on the factors cited above and his expert judgement.

In particular, the Nanobodies and polypeptides of this embodiment may be used in combination with other pharmaceutically active compounds or principles that are or can be used for the prevention and/or treatment of the diseases and disorders that can be prevented or treated with the fusion proteins or constructs of this embodiment, and as a result of which a synergistic effect may or may not be obtained.

The effectiveness of the treatment regimen used according to this embodiment may be determined and/or followed in any manner known per se for the disease or disorder involved, as will be clear to the clinician. The clinician will also be able, where appropriate and or a case-by-case basis, to change or modify a particular treatment regimen, so as to achieve the desired therapeutic effect, to avoid, limit or reduce unwanted side-effects, and/or to achieve an appropriate balance between achieving the desired therapeutic effect on the one hand and avoiding, limiting or reducing undesired side effects on the other hand.

Generally, the treatment regimen will be followed until the desired therapeutic effect is achieved and/or for as long as the desired therapeutic effect is to be maintained. Again, this can be determined by the clinician.

### DETAILED DESCRIPTION OF THE INVENTION

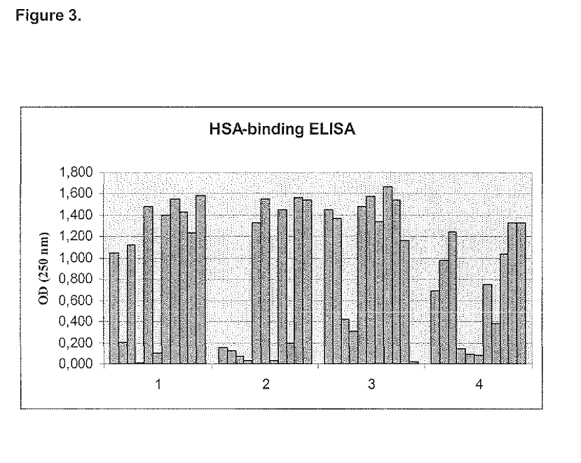

Other aspects, embodiments, advantages and applications of the invention will become clear from the further description herein, in which:
a) FIG. 1 is a non-limiting schematic drawing showing an example of the possible interaction(s) between FcRn, a serum protein binding to FcRn (such as serum albumin or IgG), a bispecific compound of the invention (in particular, a bispecific compound according to the specific embodiment for extending half-life as described herein) and an antigen (i.e. as a second intended or desired molecule). Reference is made to the further description herein.
b) FIG. 2 is a schematic drawing showing that the interaction between FcRn and a serum protein binding to FcRn is pH dependent/sensitive. Reference is made to the further description herein.
c) Tables 1-3 outline different non-limiting examples of the way in which a bispecific compound of the invention (in particular, a bispecific compound according to the specific embodiment for extending half-life as described herein) can bind to a serum protein (i.e. as a first intended or desired molecule) and to an antigen (as a second intended or desired molecule). Reference is made to the further description herein.
d) Unless indicated or defined otherwise, all terms used have their usual meaning in the art, which will be clear to the skilled person. Reference is for example made to the standard handbooks, such as Sambrook et al, “Molecular Cloning: A Laboratory Manual” (2nd. Ed.), Vols. 1-3, Cold Spring Harbor Laboratory Press (1989); F. Ausubel et al, eds., “Current protocols in molecular biology”, Green Publishing and Wiley Interscience, New York (1987); Lewin, “Genes II”, John Wiley & Sons, New York, N.Y., (1985); Old et al., “Principles of Gene Manipulation: An Introduction to Genetic Engineering”, 2nd edition, University of California Press, Berkeley, Calif. (1981); Roitt et al., “immunology” (6th. Ed.), Mosby/Elsevier, Edinburgh (2001); Roitt et al., Roitt's Essential Immunology, 10^{th} Ed. Blackwell Publishing, UK (2001); and Janeway et al., “Immunobiology” (6th Ed.), Garland Science Publishing/Churchill Livingstone, New York (2005), as well as to the general background art cited herein;
e) Unless indicated otherwise, the term “immunoglobulin sequence”—whether it used herein to refer to a heavy chain antibody or to a conventional 4-chain antibody—is used as a general term to include both the full-size antibody, the individual chains thereof, as well as all parts, domains or fragments thereof (including but not limited to antigen-binding domains or fragments such as V_{HH} domains or V_{H}/V_{L} domains, respectively). In addition, the term “sequence” as used herein (for example in terms like “immunoglobulin sequence”, “antibody sequence”, “variable domain sequence”, “V_{HH} sequence” or “protein sequence”), should generally be understood to include both the relevant amino acid sequence as well as nucleic acid sequences or nucleotide sequences encoding the same, unless the context requires a more limited interpretation;
f) Unless indicated otherwise, all methods, steps, techniques and manipulations that are not specifically described in detail can be performed and have been performed in a manner known per se, as will be clear to the skilled person. Reference is for example again made to the standard handbooks and the general background art mentioned herein and to the further references cited therein;
g) A nucleic acid sequence or amino acid sequence is considered to be “(in) essentially isolated (form)”—for example, compared to its native biological source and/or the reaction medium or cultivation medium from which it has been obtained—when it has been separated from at least one other component with which it is usually associated in said source or medium, such as another nucleic acid, another protein/polypeptide, another biological component or macromolecule or at least one contaminant, impurity or minor component. In particular, a nucleic acid sequence or amino acid sequence is considered “essentially isolated” when it has been purified at least 2-fold, in particular at least 10-fold, more in particular at least 100-fold, and up to 1000-fold or more. A nucleic acid sequence or amino acid sequence that is “in essentially isolated form” is preferably essentially homogeneous, as determined using a suitable technique, such as a suitable chromatographical technique, such as polyacrylamide-gel electrophoresis;
h) The term “domain” as used herein generally refers to a globular region of an antibody chain, and in particular to a globular region of a heavy chain antibody, or to a polypeptide that essentially consists of such a globular region. Usually, such a domain will comprise peptide loops (for example 3 or 4 peptide loops) stabilized, for example, as a sheet or by disulfide bonds;
i) The term “antigenic determinant” refers to the epitope on the antigen recognized by the antigen-binding molecule (such as a Nanobody or a polypeptide of the invention) and more in particular by the antigen-binding site of said molecule. The terms “antigenic determinant” and “epitope” may also be used interchangeably herein;
j) An amino acid sequence (such as a Nanobody, an antibody, a polypeptide of the invention, or generally an antigen binding protein or polypeptide or a fragment thereof) that can bind to, that has affinity for and/or that has specificity for a specific antigenic determinant, epitope, antigen or protein (or for at least one part, fragment or epitope thereof) is said to be “against” or “directed against” said antigenic determinant, epitope, antigen or protein;
k) The term “specificity” refers to the number of different types of antigens or antigenic determinants to which a particular antigen-binding molecule or antigen-binding protein (such as a Nanobody or a polypeptide of the invention) molecule can bind. The specificity of an antigen-binding protein can be determined based on affinity and/or avidity. The affinity, represented by the equilibrium constant for the dissociation of an antigen with an antigen-binding protein (K_{D}), is a measure for the binding strength between an antigenic determinant and an antigen-binding site on the antigen-binding protein: the lesser the value of the K_{D}, the stronger the binding strength between an antigenic determinant and the antigen-binding molecule (alternatively, the affinity can also be expressed as the affinity constant (K_{A}), which is 1/K_{D}). As will be clear to the skilled person (for example on the basis of the further disclosure herein), affinity can be determined in a manner known per se, depending on the specific antigen of interest. Avidity is the measure of the strength of binding between an antigen-binding molecule (such as a Nanobody or polypeptide of the invention) and the pertinent antigen. Avidity is related to both the affinity between an antigenic determinant and its antigen binding site on the antigen-binding molecule and the number of pertinent binding sites present on the antigen-binding molecule. Specific binding of an antigen-binding protein to an antigen or antigenic determinant can be determined in any suitable manner known per se, including, for example, Scatchard analysis and/or competitive binding assays, such as radioimmunoassays (RIA), enzyme immunoassays (EIA) and sandwich competition assays, and the different variants thereof known per se in the art.

For a general description of heavy chain antibodies and the variable domains thereof, reference is inter glia made to the following references, which are mentioned as general background art: WO 94/04678, WO 95/04079 and WO 96/34103 of the Vrije Universiteit Brussel; WO 94/25591, WO 99/37681, WO 00/40968, WO 00/43507, WO 00/65057, WO 01/40310, WO 01/44301, EP 1134231 and WO 02/48193 of Unilever; WO 97/49805, WO 01/21817, WO 03/035694, WO 03/054016 and WO 03/055527 of the Vlaams Instituut voor Biotechnologie (VIB); WO 03/050531 of Algonomics N.V. and applicant; WO 01/90190 by the National Research Council of Canada; WO 03/025020 (=EP 1 433 793) by the Institute of Antibodies; as well as WO 04/041867, WO 04/041862, WO 04/041865, WO 04/041863, WO 04/062551 by applicant and the further published patent applications by applicant; Hamers-Casterman et al., Nature 1993 Jun. 3; 363 (6428): 446-8; Davies and Riechmann, FEBS Lett. 1994 Feb. 21; 339(3): 285-90; Muyldermans et al., Protein Eng. 1994 September; 7(9): 1129-3; Davies and Riechmann, Biotechnology (NY) 1995 May; 13(5): 475-9; Gharoudi et al., 9th Forum of Applied Biotechnology, Med. Fac. Landbouw Univ. Gent. 1995; 60/4a part I: 2097-2100; Davies and Riechmann, Protein Eng. 1996 June; 9(6): 531-7; Desmyter et al., Nat Struct Biol. 1996 September; 3(9): 803-11; Sheriff et al., Nat Struct Biol. 1996 September; 3(9): 733-6; Spinelli et al., Nat. Struct Biol. 1996 September; 3(9): 752-7; Arbabi Ghahroudi et al., FEBS Lett. 1997 Sep. 15; 414(3): 521-6; Vu et al., Mol. Immunol. 1997 November-December; 34(16-17): 1121-31; Atarhouch et al., Journal of Camel Practice and Research 1997; 4: 177-182; Nguyen et al., J. Mol. Biol. 1998 Jan. 23; 275(3): 413-8; Lauwereys et al., EMBO J. 1998 Jul. 1; 17(13): 3512-20; Frenken et al., Res Immunol. 1998 July-August; 149(6):589-99; Transue et al., Proteins 1998 Sep. 1; 32(4): 515-22; Muyldermans and Lauwereys, J. Mol. Recognit. 1999 March-April; 12 (2): 131-40; van der Linden et al., Biochim. Biophys. Acta 1999 Apr. 12; 1431(1): 37-46; Decanniere et al., Structure Fold. Des. 1999 Apr. 15; 7(4): 361-70; Nguyen et al., Mol. Immunol. 1999 June; 36(8): 515-24; Woolven et al., Immunogenetics 1999 October; 50 (1-2): 98-101; Riechmann and Muyldermans, J. Immunol. Methods 1999 Dec. 10; 231 (1-2): 25-38; Spinelli et al., Biochemistry 2000 Feb. 15; 39(6): 1217-22; Frenken et al., J. Biotechnol. 2000 Feb. 28; 78(1): 11-21; Nguyen et al., EMBO J. 2000 Mar. 1; 19(5): 921-30; van der Linden et al., J. Immunol. Methods 2000 Jun. 23; 240 (1-2): 185-95; Decanniere et al., J. Mol. Biol. 2000 Jun. 30; 300 (1): 83-91; van der Linden et al., J. Biotechnol. 2000 Jul. 14; 80(3): 261-70; Harmsen et al., Mol. Immunol. 2000 August; 37(10): 579-90; Perez et al., Biochemistry 2001 Jan. 9; 40(1): 74-83; Conrath et al., J. Biol. Chem. 2001 Mar. 9; 276 (10): 7346-50; Muyldermans et al., Trends Biochem Sci. 2001 April; 26(4):230-5; Muyldermans S., J. Biotechnol. 2001 June; 74 (4): 277-302; Desmyter et al., J. Biol. Chem. 2001 Jul. 13; 276 (28): 26285-90; Spinelli et al., J. Mol. Biol. 2001 Aug. 3; 311 (1): 123-9; Conrath et al., Antimicrob Agents Chemother. 2001 October; 45 (10): 2807-12; Decanniere et al., J. Mol. Biol. 2001 Oct. 26; 313(3): 473-8; Nguyen et al., Adv Immunol. 2001; 79: 261-96; Muruganandam et al., FASEB J. 2002 February; 16 (2): 240-2; Ewert et al., Biochemistry 2002 Mar. 19; 41 (11): 3628-36; Dumoulin et al., Protein Sci. 2002 March; 11 (3): 500-15; Cortez-Retamozo et al., Int. J. Cancer. 2002 Mar. 20; 98 (3): 456-62; Su et al., Mol. Biol. Evol. 2002 March; 19 (3): 205-15; van der Vaart J M., Methods Mol. Biol. 2002; 178: 359-66; Vranken et al., Biochemistry 2002 Jul. 9; 41 (27): 8570-9; Nguyen et al., Immunogenetics 2002 April; 54 (1): 39-47; Renisio et al., Proteins 2002 Jun. 1; 47 (4): 546-55; Desmyter et al., J. Biol. Chem. 2002 Jun. 28; 277 (26): 23645-50; Ledeboer et al., J. Dairy Sci. 2002 June; 85 (6): 1376-82; De Genst et al., J. Biol. Chem. 2002 Aug. 16; 277 (33): 29897-907; Ferrat et al., Biochem. J. 2002 Sep. 1; 366 (Pt 2): 415-22; Thomassen et al., Enzyme and Microbial Technol. 2002; 30: 273-8; Harmsen et al., Appl. Microbiol. Biotechnol. 2002 December; 60 (4): 449-54; Jobling et al., Nat. Biotechnol. 2003 January; 21 (1): 77-80; Conrath et al., Dev. Comp. Immunol. 2003 February; 27 (2): 87-103; Pleschberger et al., Bioconjug. Chem. 2003 March-April; 14 (2): 440-8; Lah et al., J. Biol. Chem. 2003 Apr. 18; 278 (16): 14101-11; Nguyen et al., Immunology. 2003 May; 109 (1): 93-101; Joosten et al., Microb. Cell Fact. 2003 Jan. 30; 2 (1): 1; Li et al., Proteins 2003 Jul. 1; 52 (1): 47-50; Loris et al., Biol. Chem. 2003 Jul. 25; 278 (30): 28252-7; van Koningsbruggen et al., J. Immunol. Methods. 2003 August; 279 (1-2): 149-61; Dumoulin et al., Nature. 2003 Aug. 14; 424 (6950): 783-8; Bond et al., J. Mol. Biol. 2003 Sep. 19; 332 (3): 643-55; Yau et al., J. Immunol. Methods. 2003 Oct. 1; 281 (1-2): 161-75; Dekker et al., J. Virol. 2003 November; 77 (22): 12132-9; Meddeb-Mouelhi et al., Toxicon. 2003 December; 42 (7): 785-91; Verheesen et al., Biochim. Biophys. Acta 2003 Dec. 5; 1624 (1-3): 21-8; Zhang et al., J Mol. Biol. 2004 Jan. 2; 335 (1): 49-56; Stijlemans et al., J Biol. Chem. 2004 Jan. 9; 279 (2): 1256-61; Cortez-Retamozo et al., Cancer Res. 2004 Apr. 15; 64 (8): 2853-7; Spinelli et al., FEBS Lett. 2004 Apr. 23; 564 (1-2): 35-40; Pleschberger et al., Bioconjug. Chem. 2004 May-June; 15 (3): 664-71; Nicaise et al., Protein Sci. 2004 July; 13 (7): 1882-91; Omidfar et al., Tumour Biol. 2004 July-August; 25 (4): 179-87; Omidfar et al., Tumour Biol. 2004 September-December; 25(5-6): 296-305; Szynol et al., Antimicrob Agents Chemother. 2004 September; 48(9):3390-5; Saerens et al., J. Biol. Chem. 2004 Dec. 10; 279 (50): 51965-72: De Genst et al., J. Biol. Chem. 2004 Dec. 17; 279 (51): 53593-601; Dolk et al., Appl. Environ. Microbiol. 2005 January; 71(1): 442-50; Joosten et al., Appl Microbiol Biotechnol. 2005 January; 66(4): 384-92; Dumoulin et al., J. Mol. Biol. 2005 Feb. 25; 346 (3): 773-88; Yau et al., J Immunol Methods. 2005 February; 297 (1-2): 213-24; De Genst et al., J. Biol. Chem. 2005 Apr. 8; 280 (14): 14114-21; Huang et al., Eur. J. Hum. Genet. 2005 Apr. 13; Dolk et al., Proteins. 2005 May 15; 59 (3): 555-64; Bond et al., J. Mol. Biol. 2005 May 6; 348(3):699-709; Zarebski et al., J. Mol. Biol. 2005 Apr. 21; [E-publication ahead of print].

In accordance with the terminology used in the above references, the variable domains present in naturally occurring heavy chain antibodies will also be referred to as “V_{HH} domains”, in order to distinguish them from the heavy chain variable domains that are present in conventional 4-chain antibodies (which will be referred to hereinbelow as “V_{H} domains”) and from the light chain variable domains that are present in conventional 4-chain antibodies (which will be referred to hereinbelow as “V_{L} domains”).

As mentioned in the prior art referred to above, V_{HH} domains have a number of unique structural characteristics and functional properties which make isolated V_{HH} domains (as well as Nanobodies based thereon, which share these structural characteristics and functional properties with the naturally occurring V_{HH} domains) and proteins containing the same highly advantageous for use as functional antigen-binding domains or proteins. In particular, and without being limited thereto, V_{HH} domains (which have been “designed” by nature to functionally bind to an antigen without the presence of, and without any interaction with, a light chain variable domain) and Nanobodies can function as a single, relatively small, functional antigen-binding structural unit, domain or protein. This distinguishes the V_{HH} domains from the V_{H} and V_{L} domains of conventional 4-chain antibodies, which by themselves are generally not suited for practical application as single antigen-binding proteins or domains, but need to be combined in some form or another to provide a functional antigen-binding unit (as in for example conventional antibody fragments such as Fab fragments; in ScFv fragments, which consist of a V_{H} domain covalently linked to a V_{L} domain).

Because of these unique properties, the use of V_{HH} domains and Nanobodies as single antigen-binding proteins or as antigen-binding domains (i.e. as part of a larger protein or polypeptide) offers a number of significant advantages over the use of conventional V_{H} and V_{L} domains, scFvs or conventional antibody fragments (such as Fab- or F(ab′)₂-fragments):
only a single domain is required to bind an antigen with high affinity and with high selectivity, so that there is no need to have two separate domains present, nor to assure that these two domains are present in the right spatial conformation and configuration (i.e. through the use of especially designed linkers, as with scFvs);
V_{HH} domains and Nanobodies can be expressed from a single gene and require no post-translational folding or modifications;
V_{HH} domains and Nanobodies can easily be engineered into multivalent and multispecific formats (as further discussed herein);
V_{HH} domains and Nanobodies are highly soluble and do not have a tendency to aggregate (as with the mouse-derived antigen-binding domains described by Ward et al., Nature, Vol. 341, 1989, p. 544);
V_{HH} domains and Nanobodies are highly stable to heat, pH, proteases and other denaturing agents or conditions (see for example Ewert et al, supra);
V_{HH} domains and Nanobodies are easy and relatively cheap to prepare, even on a scale required for production. For example, V_{HH} domains, Nanobodies and proteins/polypeptides containing the same can be produced using microbial fermentation (e.g. as further described below) and do not require the use of mammalian expression systems, as with for example conventional antibody fragments;
V_{HH} domains and Nanobodies are relatively small (approximately 15 kDa, or 10 times smaller than a conventional IgG) compared to conventional 4-chain antibodies and antigen-binding fragments thereof, and therefore show high(er) penetration into tissues (including but not limited to solid tumors and other dense tissues) than such conventional 4-chain antibodies and antigen-binding fragments thereof;
V_{HH} domains and Nanobodies can show so-called cavity-binding properties (inter alia due to their extended CDR3 loop, compared to conventional V_{H} domains) and can therefore also access targets and epitopes not accessible to conventional 4-chain antibodies and antigen-binding fragments thereof. For example, it has been shown that V_{HH} domains and Nanobodies can inhibit enzymes (see for example WO 97/49805; Transue et al., (1998), supra; Lauwereys et. al., (1998), supra.

In the present invention, binding molecules, preferably proteins or peptides, are used that are endowed with the capacity to bind to a target and to an antigen, where target and antigen are in general two different molecules, with the binding interactions sensitive to certain conditions such that the serum half-life of the binding molecule, or of the antigen that is recognized by the binding protein or of both the antigen as well as the binding molecule is influenced by differential binding conditions in the compartment of blood circulation as compared to other compartments, either outside the blood compartment, such as the lymphatic system, or in sub-cellular compartments, such as the endosome, that are visited by the binding molecule.

In particular, the amino acid sequences of the invention are capable of undergoing interactions that are sensitive to the changing conditions when going from extracellular circulation to the intracellular endosomal compartment, e.g. during the process of pinocytosis. Examples of such ‘sensitive’ interactions are those which are pH-dependent, ionic strength-dependent, protease dependent, or volume dependent in such manner that the dependency creates a differential of preferably 10-fold or equally preferably 100-fold or 1000-fold on the apparent affinity of the interaction between the binding protein and its target, and as a consequence influences the circulation half life of the antigen. This target can be either an antigen itself, a protein circulating in the body, or a cell surface-based receptor.

According to one aspect of the invention, the conditional binder, preferably a protein or a peptide, binds directly to a chosen antigen in a sensitive manner. In a preferred embodiment the interactions is pH dependent in such manner that at physiological pH (7.2-7.4) the interaction occurs preferably 10×, 100×, 1000× more efficiently than at pH in the endosomal compartment (pH 6.0-6.5). The consequence is that the binding protein will bind the antigen while in circulation, but that in intracellular compartments, e.g. after internalization of the binding protein-antigen complex into the endosomal compartment, the antigen will detach from the binding protein. In a preferred embodiment this binding protein is a single variable domain, preferably a Nanobody or equally preferable a Dab (domain antibody). The consequence of such reduction in binding affinity is that the antigen is not any longer protected by virtue of the bound binding protein to the processes ongoing in the endosomal compartment and that it will be more susceptible to attack by proteases and changes in ionic conditions (which a binding protein when bound to the antigen could influence) thereby gearing the antigen and or the binding molecule more readily to the lysosomal route of protein or peptide degradation. This will influence the circulation half-life of antigen. A main advantage will be that there is no build-up of higher levels of complexes between antigen-binding protein e.g. as described during therapy with conventional monoclonal antibodies. Instead the antigen will be destroyed.

A pH dependence is the most important of all ‘sensitive’ binding manners. A sharply pH-dependent affinity transition from slightly basic pH (near the cell-surface) to acidic pH (pH 6.0) is an unusual feature of a protein/protein interaction. Receptor-mediated endocytosis is a process by which receptors transport ligands between the intracellular and extracellular environment, often taking advantage of the differences in pH between the cell-surface and intracellular vesicles to regulate the process (Melmann, ref 61 in Sprague et al).

In another aspect of the invention, an antigen-reactive (first) binding protein is itself linked to another (second) binding protein that recognizes a serum protein that is known to recognize the neonatal Fc receptor (FcRn) or salvage receptor. Alternatively, equally preferred, the antigen-reactive binding protein contains a second binding site, distinct from the antigen binding site, that recognizes a serum protein that is known to recognize FcRn or salvage receptor. Of peculiar importance is albumin, present at 41.8 mg/ml in human plasma (Davies and Morris, 1993), which in the course of endosomal recycling (Kim et al., 2006) is known to bind to FcRn at a site which is distinct from the site that is employed for IgG binding. Another equally important serum protein is IgG, abundantly present (11-12/mg/ml) in human serum (Waldmann and Strober).

If the antigen-reactive protein binds to the serum protein at the site that is recognized by FcRn, the antigen-binding protein complex becomes rapidly degraded upon endosomal uptake as the serum protein has lost it potential to be rescued by FcRn.

According to the present invention, the difference in binding strength of a binding protein towards albumin between conditions prevailing in plasma as compared to these of the endosomal compartment can rationalize the relation between Kd and t1/2. Albumin is present in plasma at very high concentration in all animals (32.7, 31.6, 38.7, 49.3, 26.3, 41.8 mg/ml in respectively mouse, rat, rabbit, monkey, dog and human as tabulated by Davis and Morris, 1993). Chaudhury et al. (2003) and Kim et al. (2006) have shown that albumin is constitutively endocytosed and rescued from degradation through acid dependent high affinity binding to FcRn (histocompatibility complex-related Fc receptor). FcRn recycles also IgG which is to enter the endocytic route via fluid phase pinocytosis or by receptor-mediated uptake (Ober et al., 2004). Interestingly, albumin binds to FcRn with a 1:1 stoichiometry (Chaudhury et al., 2006) in contrast to FcRn-IgG complexes which under equilibrium conditions have a 2:1 stoichiometry (Sanchez et al, 1999) although an apparent 1:1 stoichiometry as described in mouse (Popov et al., 1996) but this has been show to be related to alterations of carbohydrate moieties on mouse and may be related to non-equilibrium measurements (Sanchez et al, 1999). From the analysis of w.t. and FcRn-deficient mice, the turnover rate of albumin has been analysed in detail (Kim et al., 2006). The albumin recycling rate is very high. More precisely, the albumin recycling rate equals 31,000 nmol/day/kg with a steady state albumin production and catabolism rate of 31,000 nmol/day/kg.

During endosomal processing, a albumin or IgG, is bound FcRn under the acidic conditions of the endosome and follows the route of sorting endosomes to exocytosis as described in detail for IgG salvage (Ober et al., 2004). It should be noted that albumin and IgG bind to a different site in FcRn (Kim et al, 2006). The affinity for albumin to FcRn is about 200 times higher at acidic pH as compared to neutral pH in agreement with the proposed role of FcRn as a protecting receptor preventing FcRn bound albumin to enter the lysosomal degradation pathway. The Kd at pH6 of human albumin for human FcRn is 1.8 to 3 microM (Chaudhury et al., 2006). An increased binding of IgG to FcRn between pH 6 and neutral pH is also seen for IgG. The Kd at pH6 for a wild type IgG1 and human FcRn was found to be 2527 nM (Dall' Acqua et al., 2002). It should be noted that although FcRn binds albumin or IgG with similar affinity at acidic pH, the difference in stoichiometry between albumin-FcRn as compared to IgG-FcRn may perhaps result in an enhanced protection of IgG by FcRn in the endosomal compartment.

After a conditional amino acid sequence bound to a serum protein (such as a Nanobody bound to albumin or IgG) enters the endosomal compartment, it is subjected to the acidic pH (near pH 6) of the endosome, which leads to a decrease of affinity of the conditional amino acid sequence for albumin. In addition, a (further) reduction in binding to albumin may be accompanied by an increase in protease susceptibility.

Although the invention is not limited to a specific mechanism or explanation, it is expected that the Kd will be affected by pH if titratable moieties are involved in stabilizing the interaction with albumin or if the acidic pH induces conformational adjustments affecting Kd. According to the invention, this profoundly increases the t1/2 of the amino acid sequence of the invention (or a compound comprising the same).

Thus, according to one non-limiting aspect of the invention, a prolonged half life of an albumin binding conditional binder of the invention (or of a compound comprising the same) is obtained by either increasing its binding strength for albumin at serum pH (7.2-7.4) such under less favourable endosomal conditions the residual apparent Kd is such that only a limited fraction of the binding molecules is dissociated from albumin; and/or not increasing its binding strength for albumin at serum pH but enhancing the binding strength under endosomal conditions. Again, according to the invention, this teaching is not restricted to albumin but can be applied also to molecules binding to other serum proteins, such as IgG.

This particular aspect of the invention is depicted schematically in the non-limiting FIG. 1. It is well known that the interaction of various serum protein that bind to the FcRn is known to be pH sensitive (interaction 1 in FIG. 1). As a consequence the complex between the composite binding protein, the antigen and the serum protein, after pinocytosis and drop in pH can bind via the serum protein to the FcRn and its components are salvaged from degradation (FIG. 2).

The conditional binders of the invention are equally sensitive to the changes in conditions upon internalization, and as such influence the half-life of the bound antigen. As some specific non-limiting examples of this aspect of the invention, the interactions of this composite binding protein, antigen or serum protein (interactions 3 and 2, respectively in FIG. 1), may or may not be ‘sensitive’ towards changes in the conditions upon internalization. This are also summarized in Tables 1, 2 and 3, by means of representative but non-limiting examples in which the conditional binders no interaction at pH 6 and 100% interaction at pH 7.4, it being be understood that these principles apply to preferable interactions with represent a 10-fold, 100-fold or 1000-fold difference between both conditions. It should also be clear that pH 6.0 denotes the condition ‘acid pH’ and that this condition may also mean an endosomal pH in the range 5 to 6 as suggested by Kamei et al. (2005).

After internalization, the interaction(s) between the amino acid sequence of the invention, the serum protein and/or the antigen (as a second intended or desired compound) may be essentially unaffected, weakened or strengthened upon internalization (provide at least one of the interactions between the compound of the invention and the serum protein or the antigen is affected). If neither interactions 2 and 3 are sensitive to the changes in conditions upon internalization the complex between antigen, composite binding protein and serum protein, formed in circulation, is largely recycled due to the interaction of the serum protein with the FcRn (e.g. by interacting with sites on IgG or on serum albumin that allow interaction with FcRn).

A first non limiting example is depicted as case B in Table 1: In this case the interaction between the first binding protein and its antigen is lost upon reduction of the pH, and is released into the endosomal compartment. As a consequence the antigen itself is degraded, but the composite binding protein is rescued from degradation. Such approach can be used to avoid the build-up of composite binding protein-Ag complexes in circulation. Such complexes form a sink of the antigen which sometimes necessitate to increase drug dosage (e.g. with some anti-TNF-blockers). Another advantage is that the composite binding protein is recycled, and thus will need not as frequent injections and high dosing as molecules that are not recycled. This selective removal would recycle the drug itself (e.g. the Nanobody fusion), and allow a more efficient clearance of antigen from circulation than if the Nanobody fusion itself would be cleared. According to this example this route of efficient clearance of antigen from the circulation (but savaging the binding protein) favors the usage of binding proteins (such as e.g. Nanobodies, domain antibodies or other molecules) that are devoid of effector Fc part which via interaction with Fcgamma receptors mediates antibody dependent cell-mediated cytoxicity (ADCC) or antibody-dependent cell-mediated phagocytosis (ADCP) and associated clearance of IgG-complexes (Løvdal et al., 2000). In addition the recycling of the drug may affect positively its immunogenicity, as less of the drug will be prone to proteolytic cleavage and endosomal processing leading to MHC Class II presentation. Both features may contribute to the efficacy of the drug.

Another non-limiting example is depicted as case C in Table 1. At physiological pH there is no binding of the composite binding protein to the antigen in circulation, but there is after the pinocytosis event. Such setup is useful when specific uptake in this compartment is preferred, for example when interactions with the antigen in circulation could influence its function in a non-preferable manner (interference of the composite binding protein with the antigen function due to steric hindrance). A consequence of binding at low pH by the composite protein which itself is a long-lived molecules due to its interaction with the serum protein, is that the antigen could be protected from degradation and is rescued from degradation. As soon as it is released from the cell, however, it detaches from the composite protein and is allowed to function as independent molecules. For example such setup could be used to increase the half life of endogenously present cytokines or hormones.

In yet another non-limiting example (cases D, E and F in Table 2), the interaction between second binding protein and serum protein is reduced upon the drop of the pH from 7.4 to 6.0. As a consequence, after pinocytosis of the complex of composite binding protein-serum protein (with or without the antigen bound to it), the composite binding protein will loose binding affinity for the FcRn or salvage receptor and be destroyed in the endosomal compartment. Such interaction could be envisaged to be used if the extension of the half-life of the antigen should be limited to the size increase and recycling is not desirable (e.g. if the antigen is a bacterium or virus that is preferred to be cleared in a different manner). This approach can also be suitable for the rapid destruction of circulating antigens (cytokines, toxins). The three different cases in Table 2 depict what will happen is interaction of the composite binding protein with antigen is not sensitive to the pH change (D), or is altered between pH 7.4 and 6.0 (cases E and F).

A valuable application of Case F may be the control of the fate of the endosomal compartment via a amino acid sequence or compound of the invention or other binding molecule that targets e.g. Rab11 GTPase (Ward et al. 2005) to interfere with exocytosis or with Na,K-ATPases to enhance endosomal acidification (Rybak et al., 1997). Also, for example, the lysosomal route of degradation may be enhanced if a too high level of serum (IgG or albumin) is present in a patient related to a disease or other condition. Alternatively this application may be valuable in order to rapidly eliminate a prior administrated antibody which action should be limited to a small time window (e.g. to avoid undesired side affects of the antibody). By the administration of a composite binding protein, the binding molecule (Nanobody, domain antibody or other molecule) is prevented from rapid cleared by glomerular filtering and gets into action in the endosome at which point there is no need to remain bound to the carrier (e.g. IgG or albumin) because the intended action is anyhow the rerouting of the whole endosomal contents to the lysosomal degradation pathway.

In another non-limiting example (cases G, H and I in Table 3), the interaction between second binding protein and serum protein is increased upon the drop of the pH from 7.4 to 6.0. For example when there is little or no binding at the physiological pH of the binding protein to the serum protein, in circulation the binding protein is free to interact with antigen and this interaction is not affected by any interaction to the serum protein. The latter interaction may cause some steric hindrance, interfere with the pharmacokinetics of the complex of the antigen-composite binding protein, or interfere with the function of the antigen bound to the composite protein. After internalization of the antigen-composite binding protein complex is internalized and the pH decreases, and preferably at pH 6.0 the binding of the 2^{nd} binding site of the composite binding protein will become sufficient to salvage the composite binding protein from degradation. In such case the antigen bound to the composite binding protein can be retained (case G), or released for degradation (case H). In one last case (case I) the binding to antigen occurs only at the low pH, which may be a route to rescue intracellular protein released into the endosomal compartment due to the rescue by a composite binding protein.

In these aspects and examples of the invention, binding of the Amino acid sequence or compound of the invention itself may be sufficient by itself to induce a biased clearance of the antigen, but preferably the complex of the amino acid sequence or compound of the invention and the antigen is actively targeted to the endosomal compartment, e.g. by another Amino acid sequence or compound of the invention that recognizes a cell-surface target (preferably FcRn) that is regularly internalized and cleared via the endosomal compartment, or via recognition of a factor present in circulation that is cycled via the endosomal compartment. Preferred is that this cell-surface target is FcRn, or that the serum protein is IgG or albumin, or transferrin.

This invention in a further aspect encompasses methods to generate binding proteins to antigens and/or serum proteins that are sensitive in their interaction, e.g. to the changing environment upon internalization. Antibody-antigen interactions are known to be sometimes sensitive to changes in buffer conditions, pH and ionic strength, but most often those changes are not scored or investigated, and they are not often used to design drug therapeutics as variations are overall unpredictable.

Binding proteins with the desirable binding characteristics are found for example by screening repertoires of binding proteins for the occurrence of a sensitive interaction, e.g. by carrying out a binding assay with two representative conditions (e.g. at pH 7.4 and at pH 6.0), and the relative binding strength determined. Such strength of relative interaction can be measured with any suitable binding test including ELISA, BIAcore-based methods, Scatchard analysis etc. Such test will reveal which binding proteins display interactions that are sensitive to the chosen parameter (pH, ionic strength, temperature) and to what extend.

Conditional binders of the invention may alternatively be generated by selecting repertoires of binding proteins, e.g. from phage, ribosome, yeast or cellular libraries using conditions in the selection that will preferentially enrich for the desirable sensitivity. Incubating a phage antibody library at physiological pH and eluting the bound phage particles by only changing the pH to 6.0 for example will elute those phages with a pH-sensitive interaction. Similarly a change in ionic strength can be employed (e.g. from 150 mM to 10 mM NaCl or KCl) to identify interactions highly sensitive to these interactions. Equally important are conditions that are sensitive to the concentration of Ca²⁺. For example, Christensen et al., (2001), have observed reductions in [Ca²⁺]pino by two orders of magnitude as pH decreases from 7.2 to 6.2 in newly formed pinosomes, followed by significant increases in [Ca²⁺]pino as the pinosome matures, implying that low calcium concentration is a distinct physiological feature of early endosomes.

Conditional binding proteins with the desirable binding characteristics can further be isolated from designer protein libraries in which the putative binding site has been engineered to contain amino acid residues or sequences that are preferred in certain ‘sensitive’ interactions, e.g. histidines for pH-sensitivity. For example, it is known that the interaction between FcRn and IgG is exquisitely sensitive to pH, being reduced over 2 orders of magnitude as the pH is raised from pH 6.0 to 7.0. The main mechanistic basis of the affinity transition is the histidine content of the binding site: the imidazole side changes of histidine residues usually deprotonate over the pH range 6.0-7.0. The inclusion of histidines in the putative binding site (e.g. using oligonucleotides that preferentially incorporate this residue in the library) is predicted to yield a higher frequency of binding proteins with pH-sensitive interactions.

The terms and expressions which have been employed are used as terms of description and not of limitation, and there is no intention in the use of such terms and expressions of excluding any equivalents of the features shown and described or portions thereof, it being recognized that various modifications are possible within the scope of this embodiment.

All of the references described herein are incorporated by reference, in particular for the teaching that is referenced hereinabove.

### LEGENDS

FIG. 1. Possible interaction of the amino acid sequence of the invention.

FIG. 2. Interaction 1 is sensitive to changes in the pH.

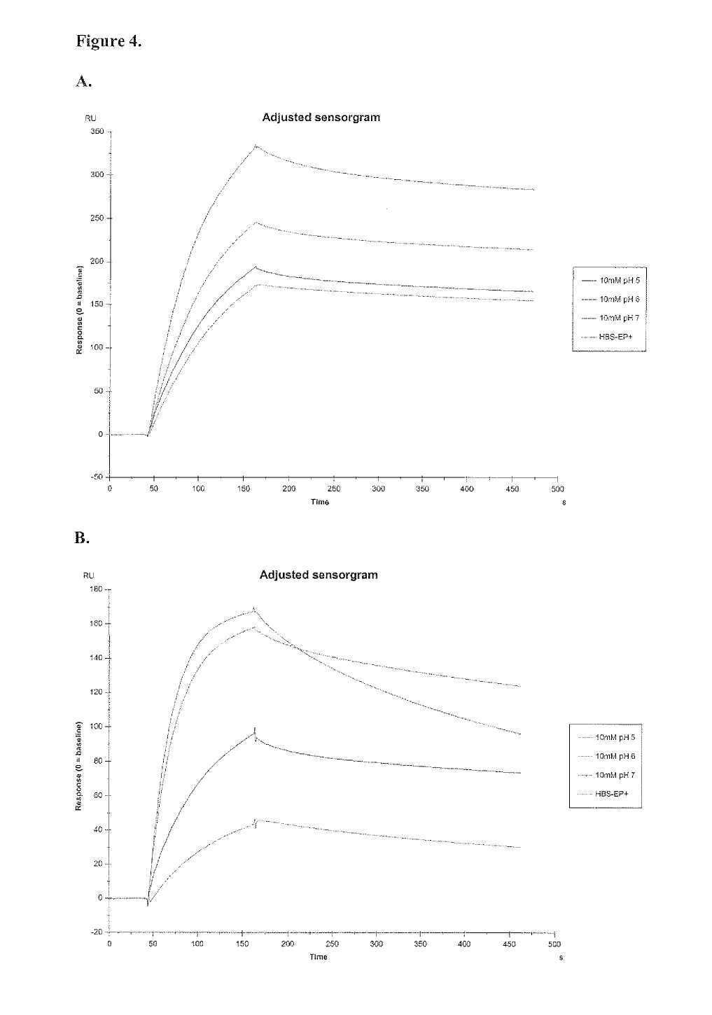

FIG. 3. Human serum albumin-specific ELISA analysis of periplasmic preparations containing his-tagged Nanobody protein fragments from selected clones. Periplasmic preparations of soluble Nanobody protein fragments are added to wells of an ELISA plate, which had been coated with HSA antigen and had been additionally blocked with PBS+1% casein. Detection is performed by a monoclonal biotinylated anti-his antibody followed by horseradish-conjugated streptavidin. The ELISA is developed by a TMB-substrate as described in Example 1. The OD-values (Y-axis) are measured at 450 nm by an ELISA-reader. Each bar represents an individual periplasmic extract.

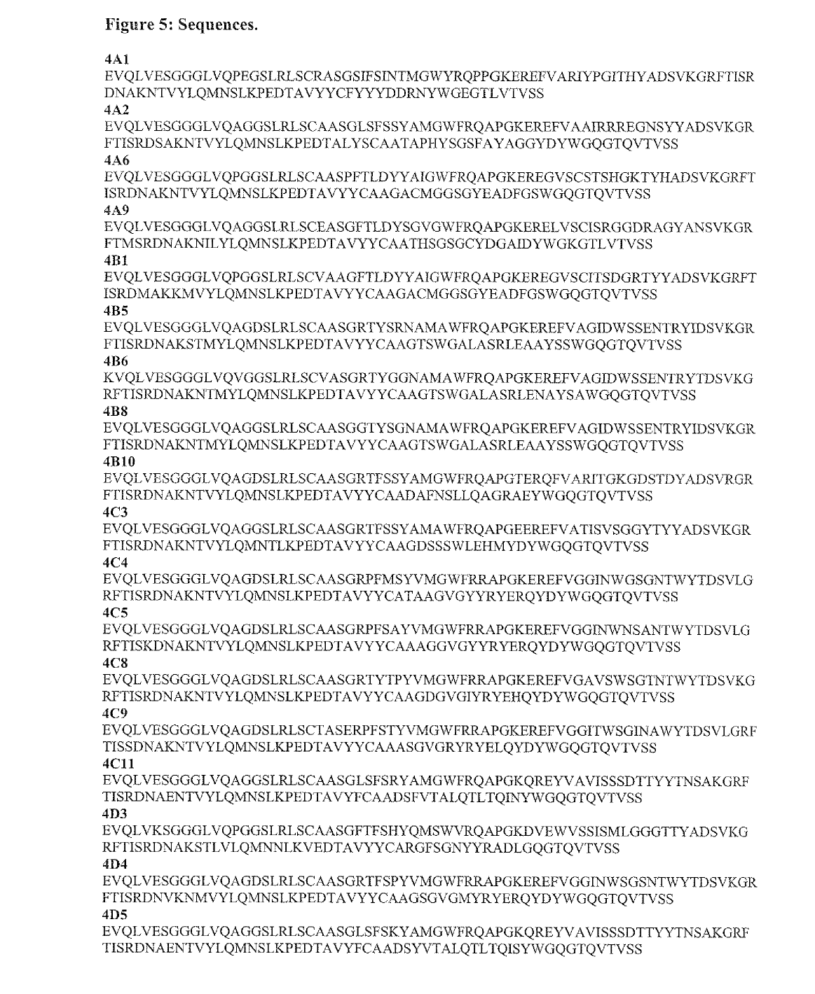

FIG. 4. Surface plasmon resonance measurements of the interaction between albumin-binding Nanobodies and human serum albumin at different pH. Periplasmic preparations of soluble Nanobody protein fragments are injected over immobilized human serum albumin at pH5, pH6 or pH7. FIGS. 4A and 4B show the interaction of nanobody 4A 1 and 4C3 respectively.

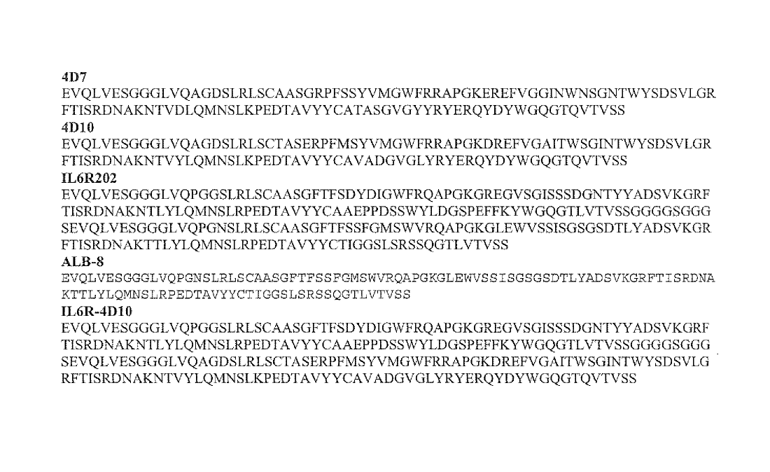

FIG. 5. Amino acid sequences.

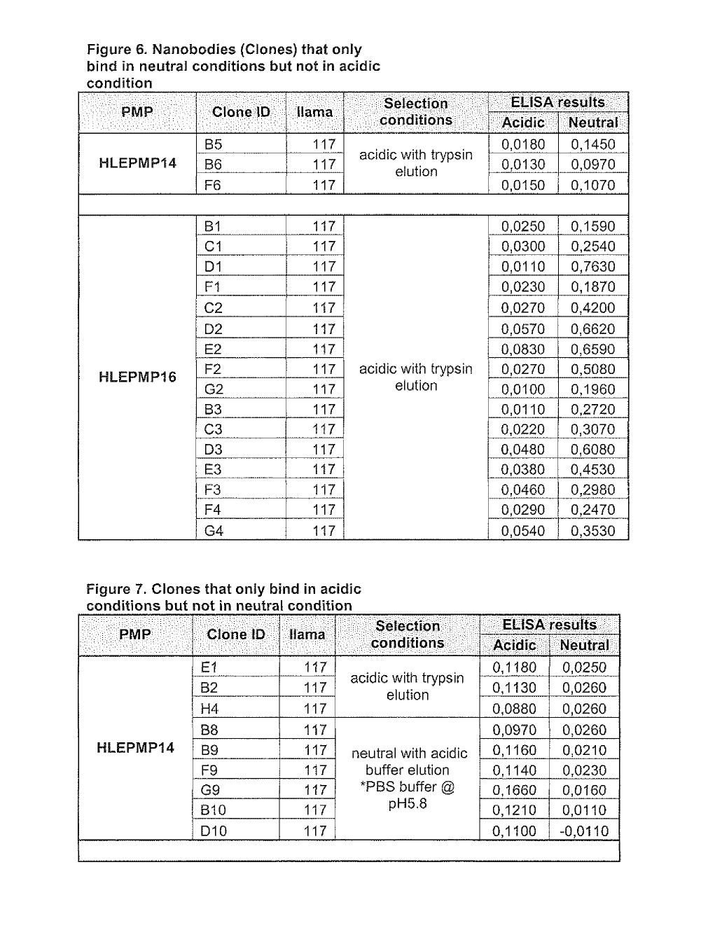

FIG. 6. Nanobodies (Clones) that only bind in neutral conditions but not in acidic conditions

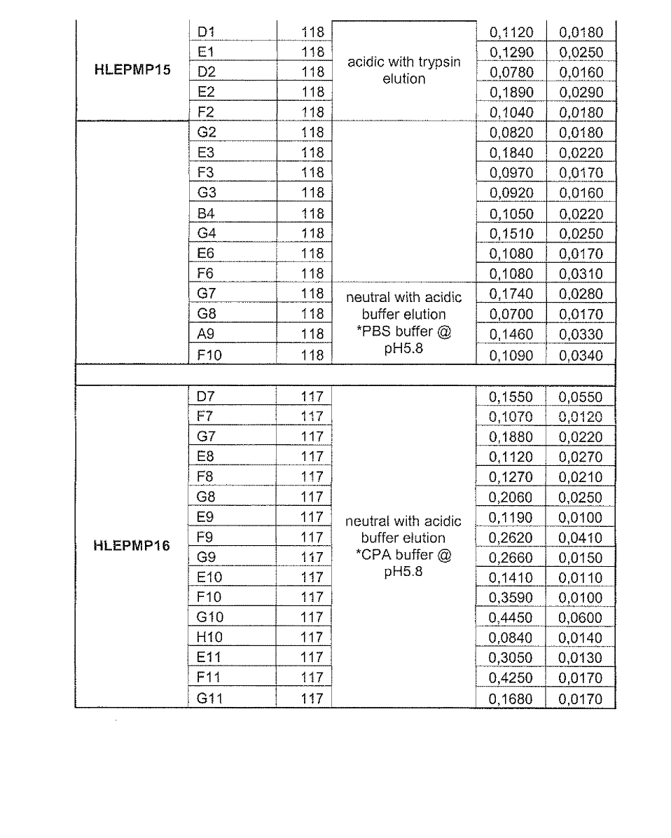

FIG. 7. Nanobodies (Clones) that only bind in acidic conditions but not in neutral conditions

### EXPERIMENTAL PART

### Example 1

### Identification of Conditional Serum Albumin Specific Nanobodies

After approval of the Ethical Committee of the Faculty of Veterinary Medicine (University Ghent, Belgium), 2 llamas (117, 118) are alternately immunized with 6 intramuscular injections at weekly interval with human serum albumin and a mixture of mouse serum albumin, cynomolgus serum albumin and baboon serum albumin, according to standard protocols.

### Library Construction

When an appropriate immune response is induced in llama, four days after the last antigen injection, a 150 ml blood sample is collected and peripheral blood lymphocytes (PBLs) are purified by a density gradient centrifugation on Ficoll-Paque™ according to the manufacturer's instructions. Next, total RNA is extracted from these cells and used as starting material for RT-PCR to amplify Nanobody encoding gene fragments. These fragments are cloned into phagemid vector pAX50. Phage is prepared according to standard methods (see for example the prior art and applications filed by applicant cited herein) and stored at 4° C. for further use.

### Selection

### Selecting Repertoires for Binding to Serum Albumin.

In a first selection, human serum albumin (Sigma A-8763) is coated onto Maxisorp 96-well plates (Nunc, Wiesbaden, Germany) at 100 μg/ml overnight (ON) at room temperature (RT). Plates are blocked with 4% Marvel in PBS for 2 h at RT. After 3 washes with PBST, phages are added in 4% Marvel/PBS and incubated for 1 h at RT. Following extensive washing, bound phage is eluted with 0.1 M triethanolamine (TEA) and neutralized with 1M Tris-HCl pH 7.5.

### Selecting Repertoires for Conditional Binding to Serum Albumin.

To enrich for conditional binders, said binders with a pH sensitive interaction, phage libraries are incubated with antigen at physiological pH and eluted at acidic pH as follows. In a first selection, human serum albumin (Sigma A-8763) is coated onto Maxisorp 96-well plates (Nunc, Wiesbaden, Germany) at 100 μg/ml overnight (ON) at room temperature (RT). Plates are blocked with 4% Marvel in PBS pH 7.3 for 2 h at RT. After 5 washes with PBS/0.05% Tween20 (PBST) pH 7.3, phages are added in 2% Marvel/PBS pH 7.3 and incubated for 2 h at RT. Unbound phages are removed by 10 washes with PBST pH7.3, followed by 2 washes with PBS pH5.8. Bound phage is eluted with PBS pH5.8 for 30 min at RT and neutralized with 1M Tris-HCl pH 7.5.

In a second selection, phage libraries are incubated for 2 h at RT with human serum albumin in 2% Marvell/CPA buffer (10 mM sodium citrate+10 mM sodium phosphate+10 mM sodium acetate+115 mM NaCl) adjusted to pH 7.3. Unbound phages are removed by 10 washes with CPA/0.05% Tween20 (CPAT) pH7.3, followed by 2 washes with CPAT pH5.8. Bound phage is eluted with CPA pH5.8 for 30 min at RT and neutralized with 1M Tris-HCl pH 7.

In a third selection strategy, phage libraries are incubated for 2 h at RT with human serum albumin in 2% Marvell/CPA pH5.8. Unbound phages are removed by 10 washes with CPAT pH5.8, followed by 2 washes with CPA pH 7.3. Bound phage is eluted with 1 mg/ml trypsin/CPA pH 7.3 for 30 min at RT.

In a fourth selection strategy, phage libraries are incubated for 2 h at RT with human scrum albumin in 2% Marvell/PBS pH5.8. Unbound phages are removed by 10 washes with PBST pH5.8, followed by 2 washes with PBSpH 7.3. Bound phage is eluted with 1 mg/ml trypsin/CPA pH 7.3 for 30 min at RT.

In all selections, enrichment is observed. The output from each selection is recloned as a pool into the expression vector pAX51. Colonies are picked and grown in 96 deep-well plates (1 ml volume) and induced by adding IPTG for Nanobody expression. Periplasmic extracts (volume: ˜80 μl) are prepared according to standard methods (see for example the prior art and applications filed by applicant cited herein).

### Library Evaluation by ELISA.

Periplasmic extracts of individual Nanobodies are screened for albumin specificity by ELISA on solid phase coated human serum albumin. Detection of Nanobody fragments bound to immobilized human serum albumin is carried out using a biotinylated mouse anti-his antibody (Serotec MCA 1396B) detected with Streptavidin-HRP (DakoCytomation #P0397). The signal is developed by adding TMB substrate solution (Pierce 34021) and detected at a wavelength of 450 min. A high hit rate of positive clones can already be obtained after panning round 1. FIG. 3 is illustrative of typical ELISA results.

Selection for Conditional or pH-Sensitive Binding of Nanobodies to Albumin by ELISA.

To enrich for conditional binders, said binders with a pH sensitive interaction, phage libraries may be incubated with antigen at physiological pH and eluted at acidic pH as follows. In a first selection strategy, human serum albumin (Sigma A-8763) is coated onto Maxisorp 96-well plates (Nunc, Wiesbaden, Germany) at 100 μg/ml overnight (ON) at room temperature (RT). Plates are blocked with 4% Marvel in PBS pH 7.3 for 2 h at RT. After 5 washes with PBS/0.05% Tween20 (PBST) pH 7.3, phages are added in 2% Marvel/PBS pH 7.3 and incubated for 2 h at RT. Unbound phages are removed by 10 washes with PBST pH7.3, followed by 2 washes with PBS pH5.8. Bound phage is eluted with PBS pH5.8 for 30 min at RT and neutralized with 1M Tris-HCl pH 7.5.

In a second selection strategy, phage libraries are incubated for 2 h at RT with human serum albumin in 2% Marvell/CPA buffer (10 mM sodium citrate+1.0 mM sodium phosphate+10 mM sodium acetate+115 mM NaCl) adjusted to pH 7.3. Unbound phages are removed by 10 washes with CPA/0.05% Tween20 (CPAT) pH7.3, followed by 2 washes with CPAT pH5.8. Bound phage is eluted with CPA pH5.8 for 30 min at RT and neutralized with 1M Tris-HCl pH 7.

In a third selection strategy, phage libraries are incubated for 2 h at RT with human serum albumin in 2% Marvell/CPA pH5.8. Unbound phages are removed by 10 washes with CPAT pH5.8, followed by 2 washes with CPA pH 7.3. Bound phage is eluted with 1 mg/ml trypsin/CPA pH 7.3 for 30 min at RT.

In a fourth selection strategy, phage libraries are incubated for 2 h at RT with human serum albumin in 2% Marvell/PBS pH5.8. Unbound phages are removed by 10 washes with PBST pH5.8, followed by 2 washes with PBSpH 7.3. Bound phage is eluted with 1 mg/ml trypsin/CPA pH 7.3 for 30 min at RT.

In all selections, enrichment is observed. The output from each selection is recloned as a pool e.g. into the expression vector pAX51. Colonies are picked and grown in 96 deep-well plates (1 ml volume) and induced by adding IPTG for Nanobody expression. Periplasmic extracts (volume: ˜80 μl) are prepared according to standard methods (see for example the prior art and applications filed by applicant cited herein).

Screening of Nanobody Repertoire for the Occurrence of a pH-Sensitive Interaction Via Surface Plasmon Resonance (BIAcore).

Human serum albumin is immobilized on a CM5 sensor chip surface via amine coupling using NHS/EDC for activation and ethanolamine for deactivation (Biacore amine coupling kit)

Approximately 1000RU of human serum albumin is immobilized. Experiments are performed at 25° C. The buffers used for the pH dependent binding of Nanobodies to albumin (Biacore) are as follows: 10 mM Sodium citrate (Na₃C₆H₅O₇)+10 mM Sodium phosphate (Na₂HPO₄)+10 mM Sodium Acetate (CH₃C00Na)+115 mM NaCl. This mixture is brought to pH7, pH6 and pH5 by adding HCl or NaOH (dependent on the pH of the mixture measured).

Periplasmic extracts are diluted in running buffers of pH7, pH6 and pH5. The samples are injected for 1 min at a flow rate of 45 ul/min over the activated and reference surfaces. Those surfaces are regenerated with a 3s pulse of glycine-HCl pH1.5+0.1% P20. Evaluation is done using Biacore T100 evaluation software.

The off rate of different Nanobodies at pH7 and pH5 is documented in Table 1. The majority of the Nanobodies (4A2, 4A6, 4B5, 4B6, 4B8, 4C3, 4C4, 4C5, 4C8, 4C9, 4D3, 4D4, 4D7 ad 4D10 have a faster off rate at pH 5 compared with pH 7 (2-6 fold difference in off rate). The Nanobody 4A9 has a slower off-rate at pH 5 compared to pH 7 (0.54 fold difference in off rate). For other Nanobodies including 4C12, 4B1, 4B10, IL6R202, Alb-8, and 4D5, binding to antigen does not change at different pH.

Direct screening of nanobody repertoires for conditional binding to antigen can thus be used.

### Screening for Conditional Binding of Nanobodies by ELISA

To screen Nanobodies for their conditional binding to albumin, a binding ELISA can also be performed with two representative conditions, pH 5.8 and pH7.3 and the relative binding strength determined. Maxisorb micro titer plates (Nunc, Article No. 430341) are coated overnight at 4° C. with 100 μl of a 1 μg/ml solution human serum albumin in bicarbonate buffer (50 mM, pH 9.6). After coating, the plates are washed three times with PBS containing 0.05% Tween20 (PBST) and blocked for 2 hours at room temperature (RT) with PBS containing 2% Marvel (PBSM). After the blocking step, the coated plates are washed 2 times with PBST pH 5.8, and a ten-fold dilution aliquot of each periplasmic sample in PBSM pH5.8 (100 μl) is transferred to the coated plates and allowed to bind for 1 hour at RT. After sample incubation, the plates are washed five times with PBST and incubated for 1 hour at RT with 100 μl of a 1:1000 dilution of mouse anti-myc antibody in 2% PBSM. After 1 hour at RT, the plates are washed five times with PBST and incubated with 100 μl of a 1:1000 dilution of a goat anti-mouse antibody conjugated with horseradish peroxidase. After 1 hour, plates are washed five times with PBST and incubated with 100 μl of slow TMB (Pierce, Article No. 34024). After 20 minutes, the reaction is stopped with 100 μl H₂SO₄. The absorbance of each well is measured at 450 nm.

92 periplasmic extracts for each of the conditional selection strategies described herein, are analyzed in this ELISA. FIG. 6 depicts the result for Nanobodies that conditionally bind to human serum albumin at neutral pH, i.e. pH 7.4, but not to acidic, i.e. pH 5.8. FIG. 7 depicts the results for Nanobodies that conditionally bind to human serum albumin at acidic pH, i.e. pH 5.8, but not to neutral pH, i.e. pH 7.4.

Upon 1 round of selection on human serum albumin, followed by total elution, Nanobodies are identified that either conditionally bind to albumin at acidic pH (n=16) or at neutral pH (n=19). Driving the selection conditions towards conditional binding, results in a higher ratio of conditionally binding nanobodies (n=23).

### Example 2

### Analysis of Effect of Conditional Binding on Pharmacokinetic Behaviour of the Nanobody

1. Construction of Bispecific Nanobody Format

Bispecific nanobodies are e.g. generated consisting of a C-terminal conditional HSA-binding Nanobody, a 9 amino acid Gly/Ser linker and an N-terminal anti-target Nanobody. These constructs may be expressed in E. coli as c-myc, His6-tagged proteins and subsequently purified from the culture medium by immobilized metal affinity chromatography (IMAC) and size exclusion chromotagraphy (SEC).

2. Retention of Conditional Binding Upon Formatting into Multispecific Format.

The conditional pH-binding properties of the anti-HSA Nanobody or dAbs within the multispecific nanobody formats are evaluated via surface plasmon resonance (BIAcore), e.g. a conditional binder as disclosed in this application is linked to one or more nanobody or dAbs binding to one or more protein target(s). Cross-reactivity to cynomolgus serum albumin is also assessed. Human and cynomolgus serum albumin are immobilized on a CM5 sensor chip surface via amine coupling using NHS/EDC for activation and ethanolamine for deactivation (Biacore amine coupling kit)

Experiments are performed at 25° C. The buffers used for the pH dependent binding of Nanobodies to albumin (Biacore) are as follows: 10 mM Sodium citrate (Na₃C₆H₅O₇)+10 mM Sodium phosphate (Na₂HPO₄)+10 mM Sodium Acetate (CH₃C00Na)+115 mM NaCl. This mixture is brought to pH7, pH6 and pH5 by adding HCl or NaOH (dependent on the pH of the mixture measured).

Purified Nanobodies are diluted in running buffers of pH7, pH6 and pH5. The samples are injected for 1 min at a flow rate of 45 ul/min over the activated and reference surfaces. Those surfaces are regenerated with a 3s pulse of glycine-HCl pH1.5+0.1% P20. Evaluation is done using Biacore T100 evaluation software.

3. Pharmacokinetic Profile of Bispecific Nanobody Formats in Cynomolgus Monkey

A pharmacokinetic study is conducted in cynomolgus monkeys. A Nanobody (e.g. IL6R-4D10, i.e. a IL-6 receptor binding block linked via a 9 amino acid Gly/Ser linker to a conditional albumin binding binding block) is administered intravenously by bolus injection (1.0 ml/kg, approximately 30 sec) in the vena cephalica of the left or right arm to obtain a dose of 2.0 mg/kg. The Nanobody concentration in the plasma samples is determined via ELISA.

The concentration in the plasma samples is determined as follows:

Maxisorb micro titer plates (Nunc, Article No. 430341) are coated overnight at 4° C. with 100 μl of a 5 μg/ml solution of 12B2-GS9-12B2 (B2#1302nr4.3.9) in bicarbonate buffer (50 mM, pH 9.6). After coating, the plates are washed three times with PBS containing 0.1% Tween20 and blocked for 2 hours at room temperature (RT) with PBS containing 1% casein (250 μl/well). Plasma samples and serial dilutions of Nanobody-standards (spiked in 100% pooled blank cynomolgus plasma) are diluted in PBS in a separate non-coated plate (Nunc, Article No. 249944) to obtain the desired concentration/dilution in a final sample matrix consisting of 10% pooled cynomolgus plasma in PBS. All pre-dilutions are incubated for 30 minutes at RT in the non-coated plate. After the blocking step, the coated plates are washed three times (PBS containing 0.1% Tween20), and an aliquot of each sample dilution (100 μl) is transferred to the coated plates and allowed to bind for 1 hour at RT. After sample incubation, the plates are washed three times (PBS containing 0.1% Tween20) and incubated for 1 hour at RT with 100 μl of a 100 ng/ml solution of sIL6R in PBS (Peprotech, Article No. 20006R). After 1 hour at RT, the plates are washed three times (PBS containing 0.1% Tween20) and incubated with 100 μl of a 250 ng/ml solution of a biotinylated polyclonal anti-IL6R antibody in PBS containing 1% casein (R&D systems, Article No. BAF227). After incubation for 30 minutes (RT), plates are washed three times (PBS containing 0.1% Tween20) and incubated for 30 minutes (RT) with 100 μl of a 1/5000 dilution (in PBS containing 1% casein) of streptavidine conjugated with horseradish peroxidase (DaktoCytomation, Article No. P0397). After 30 minutes, plates are washed three times (PBS containing 0.1% Tween20) and incubated with 100 μl of slow TMB (Pierce, Article No. 34024). After 20 minutes, the reaction is stopped with 100 μl HCl (1N). The absorbance of each well is measured at 450 nm (Tecan Sunrise spectrophotometer), and corrected for absorbance at 620 nm. This assay measures free Nanobody as well as Nanobodies bound to sIL6R and/or cynomolgus serum albumin. Concentration in each plasma sample is determined based on a sigmoidal standard curve with variable slope of the respective Nanobody.

Each individual plasma sample is analyzed in two independent assays and an average plasma concentration is calculated for pharmacokinetic data analysis.

All parameters are calculated with two-compartmental modeling, with elimination from the central compartment.

TABLE 1
pH-dependent interaction between second amino acid sequence and
antigen, but not first amino acid sequence and serum protein

| | | pH | pH | Fate | | | | |
|---|---|---|---|---|---|---|---|---|
| Case | Interaction | 6.0 | 7.4 | Nanobody | Interaction | pH 6.0 | pH 7.4 | Fate Antigen |
| B | 2 | ++ | ++ | Same | 3 | −− | ++ | Release of Ag in endosomal |
| | | | | | | | | compartment, degradation; |
| | | | | | | | | method to avoid build up of |
| | | | | | | | | Nanobody-Ag complex in |
| | | | | | | | | circulation |
| C | 2 | ++ | ++ | Same | 3 | ++ | −− | No binding to antigen in |
| | | | | | | | | circulation, useful when specific |
| | | | | | | | | uptake in this compartment is |
| | | | | | | | | preferred |

TABLE 2
Interaction between first amino acid sequence and serum protein
occurs preferentially at physiological pH

| | | pH | pH | | | pH | pH | |
|---|---|---|---|---|---|---|---|---|
| Case | Interaction | 6.0 | 7.4 | Fate Nanobody | Interaction | 6.0 | 7.4 | Fate Antigen |
| D | 2 | −− | ++ | Binding to SP in | 3 | ++ | ++ | Possibly longer half life |
| | | | | circulation; destruction of | | | | as long as in complex |
| | | | | Nanobody in endosomal | | | | with Nanobody |
| | | | | compartment; Extension | | | | |
| | | | | of half life limited to size | | | | |
| | | | | increase but no recycling | | | | |
| E | 2 | −− | ++ | Same | 3 | −− | ++ | Release of Ag in |
| | | | | | | | | endosomal |
| | | | | | | | | compartment, |
| | | | | | | | | degradation; method to |
| | | | | | | | | avoid build up of |
| | | | | | | | | Nanobody-Ag complex |
| | | | | | | | | in circulation |
| F | 2 | −− | ++ | Same | 3 | ++ | −− | Endosomal rerouting if |
| | | | | | | | | the target is e.g. Rab |
| | | | | | | | | 11 GTPase or |
| | | | | | | | | Na⁺, K⁺, ATPases. |

Please note:
pH 6.0 may mean an acid physiological pH, i.e. could also be 5.5 or less or more.
pH 7.4 may mean a neutral physiological pH, i.e. could also be a pH between 7.2 and 7.4 (and possibly a bit more or less).

TABLE 3
Preferential binding of amino acid sequence to serum protein at acidic pH

| | | pH | pH | | | pH | pH | |
|---|---|---|---|---|---|---|---|---|
| Case | Interaction | 6.0 | 7.4 | Fate Nanobody | Interaction | 6.0 | 7.4 | Fate Antigen |
| G | 2 | ++ | −− | Binding to serum | 3 | ++ | ++ | No interference of serum |
| | | | | protein in endosomal | | | | protein binding with function |
| | | | | compartment (at low | | | | of Nanobody while in |
| | | | | pH) only; upon | | | | circulation |
| | | | | release of serum | | | | |
| | | | | protein, also | | | | |
| | | | | Nanobody detaches; | | | | |
| | | | | Extension of half life | | | | |
| | | | | limited to recycling | | | | |
| | | | | effect; Advantage to | | | | |
| | | | | retain size | | | | |
| H | 2 | ++ | −− | Same | 3 | −− | ++ | Release of bound Ag in |
| | | | | | | | | endosomal compartment. |
| I | 2 | ++ | −− | Same | 3 | ++ | −− | Capture of Ag only when co- |
| | | | | | | | | pinocytosed by cells or when |
| | | | | | | | | introduced by the cell itself; |
| | | | | | | | | for specific applications this |
| | | | | | | | | could be useful |

Please note:
pH 6.0 may mean an acid physiological pH, i.e. could also be 5.5 or less or more.
pH 7.4 may mean a neutral physiological pH, i.e. could also be a pH between 7.2 and 7.4 (and possibly a bit more or less).

TABLE 4
Off rate (determined by Biacore) of different
Nanobodies ® at pH 7 and pH 5 is documented

| Nanobody | kd (1/s) at pH 7 | kd (1/s) at pH 5 | Ratio pH 7/pH 5 |
|---|---|---|---|
| 4D10 | 5.23E−04 | 3.41E−03 | 6.52 |
| 4A6 | 1.73E−03 | 9.99E−03 | 5.77 |
| 4C9 | 4.41E−04 | 1.71E−03 | 3.88 |
| 4A2 | 6.42E−03 | 2.27E−02 | 3.54 |
| 4C8 | 6.24E−04 | 2.09E−03 | 3.35 |
| 4C3 | 1.12E−03 | 3.75E−03 | 3.35 |
| 4B6 | 3.68E−04 | 1.19E−03 | 3.23 |
| 4D4 | 6.02E−03 | 1.66E−02 | 2.76 |
| 4C5 | 5.41E−04 | 1.32E−03 | 2.44 |
| 4B8 | 7.41E−04 | 1.80E−03 | 2.43 |
| 4C4 | 4.99E−04 | 1.21E−03 | 2.42 |
| 4D3 | 5.65E−03 | 1.37E−02 | 2.42 |
| 4D7 | 6.53E−04 | 1.58E−03 | 2.42 |
| 4B5 | 1.74E−03 | 4.03E−03 | 2.32 |
| 4D5 | 2.04E−02 | 2.63E−02 | 1.29 |
| 4C11 | 2.63E−02 | 3.12E−02 | 1.19 |
| 4B1 | 8.75E−03 | 7.73E−03 | 0.88 |
| 4B10 | 4.99E−02 | 4.34E−02 | 0.87 |
| 4A9 | 1.30E−02 | 7.01E−03 | 0.54 |
| Alb8 | 2.97E−03 | 2.78E−03 | 1.07 |
| IL-6R202 | 4.08E−03 | 6.19E−03 | 1.52 |

1. Amino acid sequence that is directed against a desired molecule, wherein said amino acid sequence:
a) binds to said desired molecule under a first biological condition with a dissociation constant (K_{D}) of 10⁻⁵ moles/liter or less and/or with a binding affinity (K_{A}) of at least 10⁵ M⁻¹; and
b) binds to said desired molecule under a second biological condition with a dissociation constant (K_{D}) that is at least 10 fold more than the dissociation constant with which said amino acid sequence binds to said desired molecule under said first biological condition.

2. (canceled)

3. Amino acid sequence according to claim 1, wherein said amino acid sequence binds to said desired molecule under said second biological condition with a dissociation constant (K_{D}) that is at least 1000 fold more than the dissociation constant with which said amino acid sequence binds to said desired molecule under said first biological condition.

4.-5. (canceled)

6. Amino acid sequence according to claim 1, wherein said amino acid sequence binds to said desired molecule under said first biological condition with a dissociation constant (K_{D}) of 10⁻⁸ moles/liter or less.

7.-8. (canceled)

9. Amino acid sequence according to claim 1, wherein said amino acid sequence binds to said desired molecule under said second biological condition with a dissociation constant (K_{D}) of 10⁻⁴ moles/liter or more.

10. Amino acid sequence according to claim 1, wherein said first biological condition comprises the physiological conditions prevalent in a first physiological compartment or fluid, and said second biological condition comprises the physiological conditions prevalent in a second physiological compartment or fluid, wherein the first and second physiological compartments are, under normal physiological conditions, separated by at least one biological membrane such as a cell membrane, a wall of a cellular vesicle or a subcellular compartment, or a wall of a blood vessel.

11.-13. (canceled)

14. Amino acid sequence according to claim 1, wherein said first biological condition comprises the conditions prevalent in the blood stream or the lymphatic system of a human or animal body, and said second biological condition comprises the physiological conditions prevalent in at least one subcellular compartment of a cell of said human or animal body.

15.-18. (canceled)

19. Amino acid sequence according to claim 1, wherein said amino acid sequence is directed against a serum protein that is subject to recycling, wherein the first biological condition comprises the conditions that are prevalent in the circulation of a animal or human body, and wherein the second biological condition comprises the conditions that are prevalent inside the at least one cell of the animal or human body that is involved in recycling of the serum protein.

20. Amino acid sequence according to claim 1, wherein said amino acid sequence is directed against serum albumin, wherein the first biological condition comprises the conditions that are prevalent in the circulation of an animal or human body, and wherein the second biological condition comprises the conditions that are prevalent inside at least one cell of the animal or human body that is involved in recycling of serum albumin.

21.-23. (canceled)

24. Amino acid sequence according to claim 1, wherein said first biological condition and said second biological condition differ in respect of any one, any two, any three or essentially all of the following factors: pH, ionic strength, and protease dependency.

25.-26. (canceled)

27. Amino acid sequence according to claim 1, wherein said first biological condition is a physiological pH of more than 7.2, and said second biological condition is a physiological pH of less than 6.5.

28.-29. (canceled)

30. Amino acid sequence according to claim 1, which is directed against a serum protein, in particular a human serum protein, that is subject to recycling.

31.-40. (canceled)

41. Amino acid sequence according to claim 1, which is chosen from the group consisting of heavy chain variable domains, light chain variable domains, domain antibodies and proteins and peptides suitable for use as domain antibodies, single domain antibodies and proteins and peptides suitable for use as single domain antibodies, Nanobodies® and dAbs™; or from suitable parts, fragments, analogs, homologs, orthologs, variants or derivatives of any of the foregoing.

42.-44. (canceled)

45. Compound comprising the amino acid sequence of claim 1, wherein said compound further comprises at least one further moiety or binding unit.

46.-57. (canceled)

58. Compound according to claim 45, which is a fusion protein or construct wherein in said fusion protein or construct the amino acid sequence is either directly linked to at least one therapeutic moiety or is linked to at least one therapeutic moiety via a linker or spacer (and/or incorporates at least one therapeutic moiety), in which the therapeutic moiety comprises a (single) domain antibody or a Nanobody®.

59.-61. (canceled)

62. Nucleotide sequence or nucleic acid that encodes the amino acid sequence according to claim 1.

63. Hosts or host cells that contain a nucleotide sequence or nucleic acid according to claim 62.

64. Method for preparing an amino acid sequence which method comprises cultivating or maintaining a host cell according to claim 63 under conditions such that said host cell produces or expresses the amino acid sequence so produced.

65. Amino acid sequence that is directed against a serum protein, wherein said amino acid sequence:
a) binds to said serum protein under a first biological condition with a dissociation constant (K_{D}) of 10⁻⁵ moles/liter or less and/or with a binding affinity (K_{A}) of at least 10⁵ M⁻¹; and
b) binds to said serum protein under a second biological condition with a dissociation constant (K_{D}) that is at least 10 fold more than the dissociation constant with which said amino acid sequence binds to said serum protein under said first biological condition.

66.-81. (canceled)

82. Amino acid sequence according to claim 65, wherein said amino acid sequence is directed against a serum protein that is subject to recycling, wherein the first biological condition comprises the conditions that are prevalent in the circulation of an animal or human body, and wherein the second biological condition comprises the conditions that are prevalent inside the at least one cell of the animal or human body that is involved in recycling of the serum protein.

83.-161. (canceled)
